(19) United States
(12) Patent Application Publication
(10) Pub. No.: US 2010/0261719 A1
(43) Pub. Date: Oct. 14, 2010

(54) CHEMICAL COMPOUNDS

(76) Inventors: Gregory Steven Basarab, Waltham, MA (US); Jacques Dumas, Waltham, MA (US); Pamela Hill, Waltham, MA (US)

Correspondence Address: ASTRAZENECA R&D BOSTON, 35 GATEHOUSE DRIVE, WALTHAM, MA, 02451-1215, US

(21) Appl. No.: 12/665,128

(22) PCT Filed: Jul. 1, 2008
(86) PCT No.: PCT/GB08/50530

§ 371 Date: May 10, 2010

Related U.S. Application Data
(60) Provisional application No. 60/947,654, filed on Jul. 2, 2007.

(51) Int. Cl. A61K 31/5383; C07D 498/20; A61P 31/04

(52) U.S. Cl. 514/230.5; 544/231

(57) ABSTRACT

In one aspect, the present invention relates to compounds of Formula (I) and to pharmaceutically acceptable salts thereof, wherein: n is 1 to 4; and R³ in each occurrence is independently selected from —X—R⁵, —W—R⁶, —C(O)—N(R^{3a})—S(O)₂—R^{3b}, —C(R^{3a})═N—R^{3y}, C(R^{3a})═N—N(R^{3a})—C(O)—R^{3b}, C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})2, —C(NR^{3a})₂)═N—R^{3y}, —C(N(R^{3a})₂)═N—OR^{3y}, —C(N(R^{3a})₂)═N—C(O)—R^{3b}, —C(N(R^{3a})₂═N—S(0)₂—R^{3b}, —C(N(R^{3a})₂)═N—CN, —N═C(R^{3y})₂, —N(R^{3a})—S(O)₂—N(R^{3y})₂, —N(R^{3a})—N(R^{3y}₂, —N(R^{3a})—C(O)—N(R^{3y})₂, —N(R^{3a})—C(O)—N(R^{3a})—S(O)₂—R^{3b}, —N(R^{3a})—C(R^{3a})═N(R^{3y}), —N(R^{3a})—C(R^{3a})═N—OR^{3y}, —N(R^{3a})—C(R^{3a})═N—C(O)—R^{3b}, —N(R^{3a})—C(R^{3a})═N—S(O)₂R—^{3b}, —N(R^{3a})—C(R^{3a})═N—CN, —N(R^{3a})—C(N(R^{3a})₂)═N—R^{3y}, —N(R^{3a})—C(N(R^{3a})₂)═N—OR^{3y}, —N(R^{3a})—C(N(R^{3a})₂)═N—C(O)—R^{3b}, —N(R^{3a})—C(N(R^{3a})₂)—N—S(O)₂—R^{3b}, —N(R^{3a})—C(N(R^{3a})₂)—N—CN, —O—C(O)—R³, and —Si(R^{3b})₃; to methods of using them to treat bacterial infections, and to methods for their preparation.

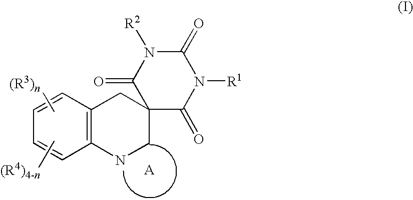

The present invention relates to novel substituted heterocycles, their pharmaceutical compositions and methods of use. In addition, the present invention relates to therapeutic methods for the treatment of bacterial infections.

The international microbiological and infectious disease community continues to express serious concern that the continuing evolution of antibacterial resistance could result in bacterial strains against which currently available antibacterial agents will be ineffective. The outcome of such an occurrence could have considerable morbidity and mortality. In general, bacterial pathogens may be classified as either Gram-positive or Gram-negative pathogens. Antibiotic compounds with effective activity against both Gram-positive and Gram-negative pathogens are generally regarded as having a broad spectrum of activity.

Gram-positive pathogens are of particular concern because of the development of resistant strains that are both difficult to treat and difficult to eradicate from the hospital environment once established. Examples of such strains are methicillin resistant Staphylococcus aureus (MRSA), methicillin resistant coagulase-negative staphylococci (MRCNS), penicillin resistant Streptococcus pneumoniae and multiple resistant Enterococcus faecium. Resistance is increasing at a steady rate rendering many agents less effective in the treatment of Gram-positive pathogens. In addition, there is increasing resistance to agents such as β-lactams, quinolones and macrolides used for the treatment of upper respiratory tract infections caused by Gram-negative strains including H. influenzae and M. catarrhalis. In addition, nosocomial Gram-negative pathogens, such as Pseudomonas aeruginosa, are difficult to treat due to resistance development. Consequently, in order to overcome the threat of widespread multi-drug resistant organisms, there is an on-going need to develop new antibacterials.

Deoxyribonucleic acid (DNA) gyrase is a member of the type II family of topoisomerases that control the topological state of DNA in cells (Champoux, J. J.; 2001. Ann Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

Antibacterials targeting DNA gyrase are well established in the art, including examples such as the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

The present invention relates to compounds of Formula (I):

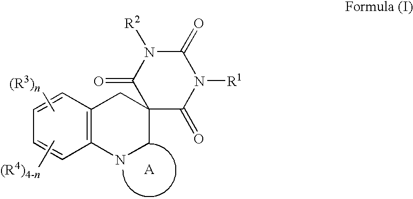

and to pharmaceutically acceptable salts thereof, wherein:

R¹ is selected from H, C₁₋₆alkyl, carbocyclyl, heterocyclyl, —C(O)—H, —C(O)—R^{1b}, —C(O)₂R^{1c}, —C(O)—N(R^{1a})₂, —S(O)—R^{1b}, —S(O)₂—R^{1b}, —S(O)₂—N(R^{1a})₂, —C(R^{1a})═N—R^{1a}, and —C(R^{1a})═N—OR^{1a}, wherein said C₁₋₆alkyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R^{10*};

R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R^{10*};

R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R^{10*};

R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R^{10*};

R² is selected from H, C₁₋₆alkyl, carbocyclyl, heterocyclyl, —C(O)—H, —C(O)—R^{2b}, —C(O)₂R^{2c}, —C(O)—N(R^{2a})₂, —S(O)—R^{2b}, —S(O)₂—R^{2b}, —S(O)₂—N(R^{2a})₂, —C(R^{2a})═N—R^{2a}, and —C(R^{2a})═N—OR^{2a}, wherein said C₁₋₆alkyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R^{20*};

R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R^{20*};

R^{2b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R^{20*}; R^{2c} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R^{20*};

R³ in each occurrence is independently selected from —X—R⁵, —W—R⁶, —C(O)—N(R^{3a})—S(O)₂—R^{3b}, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})₂, —C(N(R^{3a})₂)═N—R^{3y}, —C(N(R^{3a})₂)═N—OR^{3y}, —C(N(R^{3a})₂)═N—C(O)—R^{3b}, —C(N(R^{3a})₂)═N—S(O)₂—R^{3b}, —C(N(R^{3a})₂)═N—CN, —N═C(R^{3y})₂, —N(R^{3a})—S(O)₂—N(R^{3y})₂, —N(R^{3a})—N(R^{3y})₂, —N(R^{3a})—C(O)—N(R^{3y})₂, —N(R^{3a})—C(O)—N(R^{3a})—S(O)₂—R^{3b}, —N(R^{3a})—C(R^{3a})═N(R^{3y}), —N(R^{3a})—C(R^{3a})═N—OR^{3y}, —N(R^{3a})—C(R^{3a})═N—C(O)—R^{3b}, —N(R^{3a})—C(R^{3a})═N—S(O)₂R^{3b}, —N(R^{3a})—C(R^{3a})═N—CN, —N(R^{3a})—C(N(R^{3a})₂)═N—R^{3y}, —N(R^{3a})—C(N(R^{3a})₂)═N—OR^{3y}, —N(R^{3a})—C(N(R^{3a})₂)═N—C(O)—R^{3b}, —N(R^{3a})—C(N(R^{3a})₂)═N—S(O)₂—R^{3b}, —N(R^{3a})—C(N(R^{3a})₂)═N—CN, —O—C(O)—R^{3b}, and —Si(R^{3b})₃; R^{3a} and R^{3y} in each occurrence are independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein if said heterocyclyl contains an —NH— moiety, that nitrogen in each occurrence is optionally and independently substituted with R^{30*};

R^{3b}) in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein if said heterocyclyl contains an —NH— moiety, that nitrogen in each occurrence is optionally and independently substituted with R^{30*};

R⁴ in each occurrence is independently selected from H, halo, —CN, C₁₋₆alkyl, C₂₋₆alkenyl, C₂₋₆alkynyl, carbocyclyl, heterocyclyl, —OR^{4d}, —SR^{4d}, —N(R^{4d})₂, —N(R^{4a})—C(O)—R^{4e}, —NO₂, —C(O)—H, —C(O)—R^{4c}, —C(O)₂R^{4d}, —C(O)N(R^{4a})(R^{4d}), —O—C(O)—N(R^{4a})(R^{4d}), —N(R^{4a})—C(O)₂R^{4d}, —S(O)—R^{4c}, —S(O)₂—R^{4e}, —S(O)₂—N(R^{4a})(R^{4d}), —N(R^{4a})—S(O)₂—R^{4e}, and —C(R^{4a})═N—OR^{4d}, wherein said C₁₋₆alkyl, C₂₋₆alkenyl, and C₂₋₆alkynyl in each occurrence are optionally and independently substituted with one or more R^{40x}, and wherein said carbocyclyl and heterocyclyl in each occurrence are optionally and independently substituted on carbon with one or more R⁴⁰, and wherein if said heterocyclyl contains an —NH— moiety, that nitrogen in each occurrence is optionally and independently substituted with R^{40*};

R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein if said heterocyclyl contains an —NH— moiety, that nitrogen in each occurrence is optionally and independently substituted with R^{40*};

R^{4d} in each occurrence is independently selected from H, C₁₋₆alkyl, carbocyclyl, and aromatic heterocyclyl, wherein said C₁₋₆alkyl, carbocyclyl, and aromatic heterocyclyl in each occurrence are optionally and independently substituted on carbon with one or more R⁴⁰, and wherein if said aromatic heterocyclyl contains an —NH— moiety, that nitrogen in each occurrence is optionally and independently substituted with R^{40*};

R^{4e} in each occurrence is selected from C₁₋₆alkyl, C₂₋₆alkenyl, C₂₋₆alkynyl, carbocyclyl, and aromatic heterocyclyl, wherein said C₁₋₆alkyl, C₂₋₆alkenyl, C₂₋₆alkynyl, carbocyclyl, and aromatic heterocyclyl in each occurrence are optionally and independently substituted on carbon with one or more R⁴⁰, and wherein if said aromatic heterocyclyl contains an —NH— moiety, that nitrogen in each occurrence is optionally and independently substituted with R^{40*};

R⁵ is selected from heterocyclyl and —Si(R^{5b})₃, wherein said heterocyclyl is optionally substituted on carbon with one or more R⁵⁰, and wherein if said heterocyclyl contains an —NH— moiety, that nitrogen in each occurrence is optionally and independently substituted with R^{50*};

R^{5b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein if said heterocyclyl contains an —NH— moiety, that nitrogen in each occurrence is optionally and independently substituted with R^{50*};

R⁶ is non-aromatic heterocyclyl, wherein said non-aromatic heterocyclyl is optionally substituted on carbon with one or more R⁶⁰, and wherein if said non-aromatic heterocyclyl contains an —NH— moiety, that nitrogen in each occurrence is optionally and independently substituted with R^{60*};

R⁷ is selected from halo, —CN, C₁₋₆alkyl, C₂₋₆alkenyl, C₂₋₆alkynyl, carbocyclyl, heterocyclyl, —OR^{7a}, —SR^{7a}, —N(R^{7a})₂, —N(R^{7a})—C(O)—R^{7b}, —N(R^{7a})—N(R^{7a})₂, —NO₂, —C(O)—H, —C(O)R^{7b}, —C(O)₂R^{7a}, —C(O)—N(R^{7a})₂, —O—C(O)—N(R^{7a})₂, —N(R^{7a})—C(O)₂R^{7a}, —N(R^{7a})—C(O)—N(R^{7a})₂, —O—C(O)—R^{7b}, —S(O)—R^{7b}, —S(O)₂—R^{7b}, —S(O)₂—N(R^{7a})₂, —N(R^{7a})—S(O)₂—R^{7b}, —C(R^{7a})═N—R^{7a}, and —C(R^{7a})═N—OR^{7a}, wherein said C₁₋₆alkyl, C₂₋₆alkenyl, C₂₋₆alkynyl, carbocyclyl, and heterocyclyl are optionally substituted on carbon with one or more R⁷⁰, and wherein if said heterocyclyl contains an —NH— moiety, that nitrogen in each occurrence is optionally and independently substituted with R^{70*};

R^{7*} in each occurrence is independently selected from C₁₋₆alkyl, carbocyclyl, heterocyclyl, —C(O)—H, —C(O)—R^{7b}, —C(O)₂R^{7c}, —C(O)—N(R^{7a})₂, —S(O)—R^{7b}, —S(O)₂—R^{7b}, —S(O)₂—N(R^{7a})₂, —C(R^{7a})═N—R^{7a}, and —C(R^{7a})═N—OR^{7a}, wherein said C₁₋₆alkyl, carbocyclyl, and heterocyclyl in each occurrence are optionally and independently substituted on carbon with one or more R⁷⁰, and wherein if said heterocyclyl contains an —NH— moiety, that nitrogen in each occurrence is optionally and independently substituted with R^{70*};

R^{7a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein if said heterocyclyl contains an —NH— moiety, that nitrogen in each occurrence is optionally and independently substituted with R^{70*};

R^{7b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁷⁰, and wherein if said heterocyclyl contains an —NH— moiety, that nitrogen in each occurrence is optionally and independently substituted with R^{70*};

R^{7c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R^{70*};

R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})—C(O)—R^{10b}, —N(R^{10a})—N(R^{10a})₂, —NO₂, —C(O)—H, —C(O)—R^{10b}, —C(O)₂R^{10a}, —C(O)—N(R^{10a})₂, —O—C(O)—N(R^{10a})₂, —N(R^{10a})—C(O)₂R^{10a}, —N(R^{10a})—C(O)—N(R^{10a})₂, —O—C(O)—R^{10b}, —S(O)—R^{10b}, —S(O)₂—R^{10b}, —S(O)₂—N(R^{10a})₂, —N(R^{10a})—S(O)₂—R^{10b}, —C(R^{10a})═N—R^{10a}, and —C(R^{10a})═N—OR^{10a}, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a}*; R^{10*} in each occurrence is independently selected from C₁₋₆alkyl, carbocyclyl, heterocyclyl, —C(O)—H, —C(O)—R^{10b}, —C(O)₂R^{10c}, —C(O)—N(R^{10a})₂, —S(O)R^{10b}, —S(O)₂R^{10b}, —S(O)₂—N(R^{10a})₂, —C(R^{10a})═N—R^{10a}, and —C(R^{10a})═N—OR^{10a}, wherein said C₁₋₆alkyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a*};

R^{10a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a*};

R^{10a} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a*};

R^{10c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a*};

R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(^{20a})₂, —N(R^{20a})—C(O)—R^{20b}, —N(R^{20a})—N(R^{20a})₂, —NO₂, —C(O)—H, —C(O)—R^{20b}, —C(O)₂R^{20a}, —C(O)—N(R^{20a})₂, —O—C(O)—N(R^{20a})₂, —(R^{20a})—C(O)₂R^{20a}, —N(R^{20a})—C(O)—N(R^{20a})₂, —O—C(O)—R^{20b}, —S(O)—R^{20b}, —S(O)₂—R^{20b}, —S(O)₂—N(R^{20a})₂, —N(R^{20a})—S(O)₂—R^{20b}, —C(R^{20a})═N—R^{20a}, and —C(R^{20a})═N—OR^{20a}, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b*};

R^{20*} in each occurrence is independently selected from C₁₋₆alkyl, carbocyclyl, heterocyclyl, —C(O)—H, —C(O)—R^{20b}, —C(O)₂R^{20c}, —C(O)—N(R^{20a})₂, —S(O)—R^{20b}, —S(O)₂—R^{20b}, —S(O)₂—N(R^{20a})₂, —C(R^{20a})═N—R^{20a}, and —C(R^{20a})═N—OR^{20a}, wherein said C₁₋₆alkyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b*};

R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b*};

R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b*};

R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b*};

R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})—C(O)—R^{30b}, —N(R^{30a})—N(R^{30a})₂, —NO₂, —C(O)H, —C(O)—R^{30b}, —C(O)₂R^{30a}, —C(O)—N(R^{30a})₂, —O—C(O)—N(R^{30a})₂, —N(R^{30a})—C(O)₂R^{30a}, —N(R^{30a})—C(O)—N(R^{30a})₂, —O—C(O)—R^{30b}, —S(O)—R^{30b}, —S(O)₂—R^{30b}, —S(O)₂—N(R^{30a})₂, —N(R^{30a})—S(O)₂—R^{30b}, —Si(R^{30b})₃, —C(R^{30a})═N—R^{30a}, and —C(R^{30a})═N—OR^{30a}, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c*};

R^{30*} in each occurrence is independently selected from C₁₋₆alkyl, carbocyclyl, heterocyclyl, —C(O)—H, —C(O)—R^{30b}, —C(O)₂R^{30c}, —C(O)—N(R^{30a})₂, —S(O)—R^{30b}, —S(O)₂—R^{30b}, —S(O)₂—N(R^{30a})₂, —C(R^{30a})═N—R^{30a}, and —C(R^{30a})═N—OR^{30a}, wherein said C₁₋₆alkyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c*};

R^{30a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c*};

R^{30b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c*};

R^{30c} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c*};

R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R^{40a})₂, —N(R^{40a})—S(O)₂—R^{40b}, —C(R^{40a})═N—R^{40a}, and —C(R^{40a})═N—OR^{40a}, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d*};

R^{40*} in each occurrence is independently selected from C₁₋₆alkyl, carbocyclyl, heterocyclyl, —C(O)—H, —C(O)R^{40b}, —C(O)₂R^{40c}, —C(O)—N(R^{40a})₂, —S(O)R^{40b}, —S(O)₂—R^{40b}, —S(O)₂—N(R^{40a})₂, —C(R^{40a})═N—R^{40a}, and —C(R^{40a})═N—OR^{40a}, wherein said C₁₋₆alkyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d*};

R^{40a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d*};

R^{40b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d*};

R^{40c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d*};

R^{40x} in each occurrence is independently selected from halo, —CN, C₁₋₆alkyl, C₂₋₆alkenyl, C₂₋₆alkynyl, carbocyclyl, —OR^{40a}, SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)—R^{40b}, —S(O)₂—N(R^{40a})₂, —N(R^{40a})—S(O)₂—R^{40b}, —C(R^{40a})═N—R^{40a}, and —C(R^{40a})═N—OR^{40a}, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d*};

R⁵⁰ in each occurrence is independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})—C(O)—R^{50b}, —N(R^{50a})—N(R^{50a})₂, —NO₂, —C(O)—H, —C(O)—R^{50b}, —C(O)₂R^{50a}, —C(O)—N(R^{50a})₂, —O—C(O)—N(R^{50a})₂, —N(R^{50a})—C(O)₂R^{50a}, —N(R^{50a})—C(O)—N(R^{50a})₂, —O—C(O)—R^{50b}, —S(O)—R^{50b}, —S(O)₂—R^{50b}, —S(O)₂—N(R^{50a})₂, —N(R^{50a})—S(O)₂—R^{50b}, —Si(R^{50b})₃, —C(R^{50a})═N(R^{50a}), and —C(R^{50a})═N(OR^{50a}), wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e*};

R^{50*} in each occurrence is independently selected from C₁₋₆alkyl, carbocyclyl, heterocyclyl, —C(O)—H, —C(O)—R^{50b}, —C(O)₂R^{50c}, —C(O)—N(R^{50a})₂, —S(O)—R^{50b}, —S(O)₂—R^{50b}, —S(O)₂—N(R^{50a})₂, —C(R^{50a})═N—R^{50a}, and —C(R^{50a})═N—OR^{50a}, wherein said C₁₋₆alkyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with Re^{*};

R^{50a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e*};

R^{50b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e*};

R^{50c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e*};

R⁶⁰ in each occurrence is independently selected from halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})—C(O)—R^{60b}, —N(R^{60a})—N(R^{60a})₂, —NO₂, —C(O)—H, —C(O)—R^{60b}, —C(O)₂R^{60a}, —C(O)—N(R^{60a})₂, —O—C(O)—N(R^{60a})₂, —N(R^{60a})—C(O)₂R^{60a}, —N(R^{60a})—C(O)—N(R^{60a})₂, —O—C(O)—R^{60b}, —S(O)—R^{60b}, —S(O)₂—R^{60b}, —S(O)₂—N(R^{60a})₂, —N(R^{60a})—S(O)₂—R^{60b}, —C(R^{60a})═N—R^{60a}, and —C(R^{60a})═N—OR^{60a}, wherein said C₁₋₆alkyl, C₂₋₆alkenyl, C₂₋₆alkynyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f};

R^{60*} in each occurrence is independently selected from C₁₋₆alkyl, carbocyclyl, heterocyclyl, —C(O)—H, —C(O)—R^{60b}, —C(O)₂R^{60c}, —C(O)—N(R^{60a})₂, —S(O)—R^{60b}, —S(O)₂—R^{60b}, —S(O)₂—N(R^{60a})₂, —C(R^{60a})═N—R^{60a}, and —C(R^{60a})═N—OR^{60a}, wherein said C₁₋₆alkyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f*};

R^{60a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{f*};

R^{60b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f*};

R^{60c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f*};

R⁷⁰ in each occurrence is independently selected from halo, —CN, C₁₋₆alkyl, C₂₋₆alkenyl, C₂₋₆alkynyl, carbocyclyl, heterocyclyl, —OR^{70a}, —SR^{70a}, —N(R^{70a})₂, —N(R^{70a})—C(O)—R^{70b}, —N(R^{70a})—N(R^{70a})₂, —NO₂, —C(O)—H, —C(O)—R^{70b}, —C(O)₂R^{70a}, —C(O)—N(R^{70a})₂, —O—C(O)—N(R^{70a})₂, —N(R^{70a})—C(O)₂R^{70a}, —N(R^{70a})—C(O)—N(R^{70a})₂, —O—C(O)—R^{70b}, —S(O)—R^{70b}, —S(O)₂R^{70b}, —S(O)₂—N(R^{70a})₂, —N(R^{70a})—S(O)₂—R^{70b}, —C(R^{70a})═N—R^{70a}, and —C(R^{70a})═N—OR^{70a}, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g*};

R^{70*} in each occurrence is independently selected from C₁₋₆alkyl, carbocyclyl, heterocyclyl, —C(O)—H, —C(O)—R^{70b}, —C(O)₂R^{70c}, —C(O)—N(R^{70a})₂, —S(O)—R^{70b}, —S(O)₂—R^{70b}, —S(O)₂—N(R^{70a})₂, —C(R^{70a})═N—R^{70a}, and —C(R^{70a})═N—OR^{70a}, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g*};

R^{70a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g*};

R^{70b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g*};

R^{70c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g*};

R^{a}, R^{b}, R^{c}, R^{d}, R^{e}, R^{f}, and R^{g} in each occurrence are independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})—C(O)—Rⁿ, —N(R^{m})—N(R^{m})₂, —NO₂, —C(O)—H, —C(O)—Rⁿ, —C(O)₂R^{m}, —C(O)—N(R^{m})₂, —O—C(O)—N(R^{m})₂, —N(R^{m})—C(O)₂R^{m}, —N(R^{m})—C(O)—N(R^{m})₂, —O—C(O)—Rⁿ, —S(O)—Rⁿ, —S(O)₂—Rⁿ, —S(O)₂—N(R^{m})₂, —N(R^{m})—S(O)₂—Rⁿ, —C(R^{m})═N—R^{m}, and —C(R^{m})═N—OR^{m};

R^{a*}, R^{b*}, R^{c*}, R^{d}, R^{e*}, R^{f*}, and R^{g} in each occurrence are independently selected from C₁₋₆alkyl, carbocyclyl, heterocyclyl, —C(O)—H, —C(O)—Rⁿ, —C(O)₂R^{o}, —C(O)—N(R^{m})₂, —S(O)—Rⁿ, —S(O)₂—Rⁿ, —S(O)₂—N(R^{m})₂, —C(R^{m})═N—R^{m}, and —C(R^{m})═N—OR^{m};

R^{m} in each occurrence is independently selected from H, C₁₋₆alkyl, carbocyclyl, and heterocyclyl;

Rⁿ in each occurrence is independently selected from C₁₋₆alkyl, C₂₋₆alkenyl, C₂₋₆alkynyl, carbocyclyl, and heterocyclyl;

R^{o} in each occurrence is independently selected from C₁₋₆alkyl, carbocyclyl, and heterocyclyl;

W in each occurrence is independently selected from —O—, —S—, —N(R^{3a})—, —N(R^{3a})—C(O)—, —C(O)—, —C(O)₂—, —C(O)—N(R^{3a})—, —O—C(O)—N(R^{3a})—, —N(R^{3a})—C(O)₂—, —S(O)—, —S(O)₂—, —S(O)₂—, and —N(R^{3a})—S(O)₂—;

X in each occurrence is independently selected from C₁₋₆alkylene, C₂₋₆alkenylene, and C₂₋₆alkynylene, wherein said C₁₋₆alkylene, C₂₋₆alkenylene, and C₂₋₆alkynylene, in addition to the R⁵ to which they are attached, in each occurrence is optionally and independently substituted with one or more R⁴⁰;

Ring A is a 5- to 7-membered non-aromatic heterocyclic ring, wherein
1) said 5- to 7-membered heterocyclic ring optionally contains, in addition to the nitrogen, a member selected from —O—, —NH—, and —S—;
2) said 5- to 7-membered heterocyclic ring is optionally substituted on carbon with one or more R⁷;
3) two R⁷ substituents on one carbon atom may together optionally form the group ═O or the group ═N(OR^{7a}); and
4) if said 5- to 7-membered heterocyclic ring contains an —NH— moiety, that nitrogen is optionally substituted with R^{7*}; and

n is 1 to 4.

In this specification the prefix C_{x-y} as used in terms such as C_{x-y}alkyl and the like (where x and y are integers) indicates the numerical range of carbon atoms that are present in the group; for example, C₁₋₄alkyl includes C₁alkyl (methyl), C₁alkyl (ethyl), C₁alkyl (propyl and isopropyl) and C₄alkyl (butyl, 1-methylpropyl, 2-methylpropyl, and t-butyl).

Unless specifically stated, the bonding atom of a group may be any suitable any suitable atom of that group; for example, propyl includes prop-1-yl and prop-2-yl.

Where a particular R group (e.g. R^{1a}, R¹⁰, etc.) is present in a compound of Formula (I) more than once, it is intended that each selection for that R group is independent at each occurrence of any selection at any other occurrence. For example, the —N(R)₂ group is intended to encompass: 1) those —N(R)₂ groups in which both R substituents are the same, such as those in which both R substituents are, for example, C₁₋₆alkyl; and 2) those —N(R)₂ groups in which each R substituent is different, such as those in which one R substituent is, for example, H, and the other R substituent is, for example, carbocyclyl.

With regard to divalent linker W, it is intended that for each definition provided therefor, the left-most portion of that definition's moiety is the point of attachment. For example, a compound of Formula (I) in which:

R³ is —W—R⁶;

R⁴ is H;

W is —N(R^{3a})—S(O)₂—; and

n is 1,

would have the following structure:

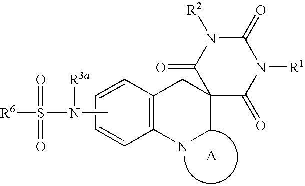

Alkyl—As used herein the term “alkyl” refers to both straight and branched chain saturated hydrocarbon radicals having the specified number of carbon atoms. For example, “C₁₋₆alkyl” includes, but is not limited to, groups such as C₁₋₃alkyl, methyl, ethyl, propyl, isopropyl, butyl, pentyl, and hexyl. References to individual alkyl groups such as “propyl” are specific for the straight chain version only and references to individual branched chain alkyl groups such as ‘isopropyl’ are specific for the branched chain version only.

Alkylene—As used herein the term “alkylene” refers to both straight and branched chain saturated hydrocarbon diradicals having the specified number of carbon atoms. For example, “C₁₋₆alkylene” includes, but is not limited to, groups such as C₁₋₃alkylene, methylene, ethylene, propylene, isopropylene, butylene, pentylene, and hexylene.

Alkenyl—As used herein, the term “alkenyl” refers to both straight and branched chain hydrocarbon radicals having the specified number of carbon atoms and containing at least one carbon-carbon double bond. For example, “C₂₋₆alkenyl” includes, but is not limited to, groups such as C₂₋₅alkenyl, ethenyl, 2-propenyl, 2-methyl-2-propenyl, 3-butenyl, 4-pentenyl, and 5-hexenyl.

Alkenylene—As used herein, the term “alkenylene” refers to both straight and branched chain hydrocarbon radicals having the specified number of carbon atoms and containing at least one carbon-carbon double bond. In one aspect, “alkeenylene” may be ethene-1,2-diyl.

Alkynyl—As used herein, the term “alkynyl” refers to both straight and branched chain hydrocarbon radicals having the specified number of carbon atoms and containing at least one carbon-carbon triple bond. For example, “C₂₋₆alkynyl” includes, but is not limited to, groups such as C₂₋₄alkynyl, ethynyl, 2-propynyl, 2-methyl-2-propynyl, 3-butynyl, 4-pentynyl, and 5-hexynyl.

Alkynylene—As used herein, the term “alkynylene” refers to both straight and branched chain hydrocarbon radicals having the specified number of carbon atoms and containing at least one carbon-carbon triple bond. In one aspect, “alkynylene” may be ethyne-1,2-diyl.

Halo—As used herein, the term “halo” is intended to include fluoro, chloro, bromo and iodo. In one aspect, the “halo” may refer fluoro, chloro, and bromo. In another aspect, “halo” may refer to fluoro and chloro. In still another aspect, “halo” may refer to fluoro. In yet another aspect, “halo” may refer to chloro.

Carbocyclyl—As used herein, the term “carbocyclyl” refers to a saturated, partially saturated, or unsaturated, mono or bicyclic carbon ring that contains 3-12 atoms, wherein one or more —CH₂— groups may optionally be replaced by a corresponding number of —C(O)— groups. In one aspect, the term “carbocyclyl” may refer to a monocyclic ring containing 5 or 6 atoms or a bicyclic ring containing 9 or 10 atoms. Illustrative examples of “carbocyclyl” include, but are not limited to, adamantyl, cyclopropyl, cyclobutyl, cyclopentyl, cyclopentenyl, cyclohexyl, cyclohexenyl, 1-oxocyclopentyl, phenyl, naphthyl, tetralinyl, indanyl or 1-oxoindanyl. In one aspect, “carbocyclyl” may be phenyl. In another aspect, “carbocyclyl” may be selected from cyclopropyl, cyclobutyl, cyclopentyl, cyclopentenyl, and cyclohexyl.

3- to 6-Membered Carbocyclyl—In one aspect, “carbocyclyl” may be “3- to 6-membered carbocyclyl.” The term “3- to 6-membered carbocyclyl” refers to a saturated or partially saturated monocyclic carbon ring containing 3 to 6 ring atoms, of which one or more —CH₂— groups may be optionally replaced with a corresponding number of —C(O)— groups. Illustrative examples of “3- to 6-membered carbocyclyl” include cyclopropyl, cyclobutyl, cyclopentyl, oxocyclopentyl, cyclopentenyl, cyclohexyl, and phenyl.

Heterocyclyl—As used herein, the term “heterocyclyl” refers to a saturated, partially saturated or unsaturated, mono or bicyclic ring containing 4 to 12 atoms of which at least one atom is selected from nitrogen, sulfur or oxygen, which may, unless otherwise specified, be carbon or nitrogen linked, wherein a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, 1,3-benzodioxolyl, 1-benzothiophenyl, 1,3-benzothiazolyl, 1,3-benzoxazolyl, dioxidotetrahydrothiophenyl, 3,5-dioxopiperidinyl, imidazolyl, indolyl, isoquinolone, isothiazolyl, isoxazolyl, morpholino, oxoimidazolidinyl, 2-oxopyrrolidinyl, 2-oxotetrahydrofuranyl, 2-oxo-1,3-thiazolidinyl, piperazinyl, piperidinyl, pyranyl, pyrazolyl, pyridinyl, pyrrolyl, pyrrolidinyl, pyrrolinyl, pyrimidyl, pyrazinyl, pyrazolyl, pyridazinyl, 4-pyridone, quinolyl, tetrazolyl, tetrahydropyranyl, tetrahydropyranyl, thiazolyl, 1,3,4-thiadiazolyl, thiazolidinyl, thienyl, thiomorpholino, 4H-1,2,4-triazolyl, pyridine-N-oxide and quinoline-N-oxide. In one aspect of the invention the term “heterocyclyl” may refer to a saturated, partially saturated, or unsaturated, monocyclic ring containing 5 or 6 atoms of which at least one atom is chosen from nitrogen, sulfur or oxygen, and may, unless otherwise specified, be carbon or nitrogen linked, and a ring nitrogen atom may be optionally oxidized to form an N-oxide.

5- or 6-Membered Heterocyclyl—In one aspect, “heterocyclyl” may be “5- or 6-membered heterocyclyl,” which refers to a saturated, partially saturated, or unsaturated, monocyclic ring containing 5 or 6 ring atoms, of which at least one ring atom is selected from nitrogen, sulfur, and oxygen, and of which a —CH₂— group may be optionally replaced by a —C(O)— group. Unless otherwise specified, “5- or 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heterocyclyl” include dioxidotetrahydrothiophenyl, 2,4-dioxoimidazolidinyl, 3,5-dioxopiperidinyl, furanyl, imidazolyl, isothiazolyl, isoxazolyl, morpholinyl, oxazolyl, oxoimidazolidinyl, 2-oxopyrrolidinyl, 2-oxotetrahydrofuranyl, oxo-1,3-thiazolidinyl, piperazinyl, piperidinyl, 2H-pyranyl, pyrazolyl, pyridinyl, pyrrolyl, pyrrolidinyl, pyrrolidinyl, pyrimidinyl, pyrazinyl, pyrazolyl, pyridazinyl, 4-pyridonyl, tetrazolyl, tetrahydrofuranyl, tetrahydropyranyl, thiazolyl, thiadiazolyl, 1,34-thiazolidinyl, thiomorpholinyl, thiophenyl, 4H-1,2,4-triazolyl, and pyridine-N-oxidyl.

5 or 6-Membered Non-Aromatic Heterocyclyl—In one aspect, “heterocyclyl” and “5- or 6-membered heterocyclyl” may be “5 or 6-membered non-aromatic heterocyclyl.” The term “5- or 6-membered non-aromatic heterocyclyl” is intended to refer to a saturated or partially saturated, monocyclic, non-aromatic heterocyclyl ring containing 5 or 6 ring atoms, of which at least one ring atom is selected from nitrogen, sulfur, and oxygen, and which may, unless otherwise specified, be carbon or nitrogen linked, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of “5 or 6-membered non-aromatic heterocyclyl” include dioxidotetrahydrothiophenyl, 2,4-dioxoimidazolidinyl, 3,5-dioxopiperidinyl, morpholinyl, oxoimidazolidinyl, 2-oxopyrrolidinyl, 2-oxotetrahydrofuranyl, oxo-1,3-thiazolidinyl, piperazinyl, piperidinyl, 2H-pyranyl, pyrrolidinyl, tetrahydrofuranyl, tetrahydropyranyl, thiomorpholinyl, and thiazolidinyl.

5- or 6-Membered Heteroaryl—In one aspect, “heterocyclyl” and “5- or 6-membered heterocyclyl” may be “5- or 6-membered heteroaryl.” The term “5- or 6-membered heteroaryl” is intended to refer to a monocyclic, aromatic heterocyclyl ring containing 5 or 6 ring atoms, of which at least one ring atom is selected from nitrogen, sulfur, and oxygen. Unless otherwise specified, “6-membered heteroaryl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heteroaryl” include furanyl, imidazolyl, isothiazolyl, isoxazole, oxazolyl, pyrazinyl, pyrazolyl, pyridazinyl, pyrimidinyl, pyridinyl, pyrrolyl, tetrazolyl, 1,3,4-thiadiazolyl, thiazolyl, thiophenyl, 4H-1,2,4-triazolyl.

5- to 7-Membered Non-Aromatic Heterocyclic Ring—For the purposes of Ring A, the term “5- to 7-membered non-aromatic heterocyclic ring” is intended to refer to a saturated or partially saturated, monocyclic, non-aromatic heterocyclic ring containing - to 7 ring atoms, which may contain, in addition to the bridgehead nitrogen shown in Formula (I), a member selected from —O—, —NH—, and —S—, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of “5- to 7-membered non-aromatic heterocyclic ring” include 3,5-dioxopiperidine, morpholine, 2-oxopyrrolidine, 2-oxotetrahydrofuranyl, oxo-1,3-thiazolidine, piperazine, piperide, 2H-pyrane, pyrrolidine, thiomorpholine, and thiazolidine. In one aspect, “5- to 7-membered non-aromatic heterocyclic ring” is morpholine.

Optionally substituted—As used herein, the phrase “optionally substituted” indicates that substitution is optional and therefore it is possible for the designated group to be either substituted or unsubstituted. In the event a substitution is desired, the appropriate number of hydrogens on the designated group may be replaced with a selection from the indicated substituents, provided that the normal valency of the atoms on a particular substituent is not exceeded, and that the substitution results in a stable compound.

In one aspect, when a particular group is designated as being optionally substituted with one or more substituents, the particular group may be unsubstituted. In another aspect, the particular group may bear one substituent. In another aspect, the particular substituent may bear two substituents. In still another aspect, the particular group may bear three substituents. In yet another aspect, the particular group may bear four substituents. In a further aspect, the particular group may bear one or two substituents. In still a further aspect, the particular group may be unsubstituted, or may bear one or two substituents.

Pharmaceutically Acceptable—As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Effective Amount—As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

Leaving Group—As used herein, the phrase “leaving group” is intended to refer to groups readily displaceable by a nucleophile such as an amine nucleophile, and alcohol nucleophile, or a thiol nucleophile. Examples of suitable leaving groups include halo, such as chloro and bromo, and sulfonyloxy group, such as methanesulfonyloxy and toluene-4-sulfonyloxy.

Protecting Group—As used herein, the term “protecting group” is intended to refer to those groups used to prevent selected reactive groups (such as carboxy, amino, hydroxy, and mercapto groups) from undergoing undesired reactions.

Illustrative examples of suitable protecting groups for a hydroxy group include, but are not limited to, an acyl group; alkanoyl groups such as acetyl; aroyl groups, such as benzoyl; silyl groups, such as trimethylsilyl; and arylmethyl groups, such as benzyl. The deprotection conditions for the above hydroxy protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

Illustrative examples of suitable protecting groups for an amino group include, but are not limited to, acyl groups; alkanoyl groups such as acetyl; alkoxycarbonyl groups, such as methoxycarbonyl, ethoxycarbonyl, and t-butoxycarbonyl; arylmethoxycarbonyl groups, such as benzyloxycarbonyl; and aroyl groups, such benzoyl. The deprotection conditions for the above amino protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron trichloride). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or during work-up.

Compounds of Formula (I) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethyl- sulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Some compounds of Formula (I) may have chiral centres and/or geometric isomeric centres (E- and Z-isomers), and it is to be understood that the invention encompasses all such optical, diastereoisomers and geometric isomers. The invention further relates to any and all tautomeric forms of the compounds of Formula (I).

It is also to be understood that certain compounds of Formula (I) can exist in solvated as well as unsolvated forms such as, for example, hydrated forms. It is to be understood that the invention encompasses all such solvated forms.

Additional embodiments of the invention are as follows. These additional embodiments relate to compounds of Formula (I) and pharmaceutically acceptable salts thereof. Such specific substituents may be used, where appropriate, with any of the definitions, claims or embodiments defined hereinbefore or hereinafter.

R¹

In one aspect, R¹ is H.

R²

In one aspect, R² is H.

_{R}³

In one aspect, R³ in each occurrence is independently selected from X—R⁵, W—R⁶, —C(H)═N—R^{3y}, —C(H)═N—(NR^{3a})—C(O)—R^{3b}, —N═C(R^{3y})₂, —N(H)—S(O)₂—N(R^{3y})₂, and —N(H)—C(O)—N(R^{3y})₂;

R^{3b} is C₁₋₆alkyl;

R^{3y} in each occurrence is independently selected from H, C₁₋₆alkyl, carbocyclyl, and heterocyclyl, wherein said C₁₋₆alkyl, carbocyclyl, and heterocyclyl are optionally substituted on carbon with one or more R³⁰, and wherein if said heterocyclyl contains an —NH— moiety, that nitrogen in each occurrence is optionally and independently substituted with R^{30*};

R^{3a} is H;

R⁶ is non-aromatic heterocyclyl;

R⁵ in each occurrence is independently selected from heterocyclyl and —Si(R^{5b})₃, wherein if said heterocyclyl contains an —NH— moiety, that nitrogen in each occurrence is optionally and independently substituted with R^{50*};

R^{5b} is C₁₋₆alkyl;

R³⁰ in each occurrence is independently selected from halo, —CN, C₁₋₆alkyl, —OR^{30a};

R^{30a} is C₁₋₆alkyl;

R³⁰* is C₁₋₆alkyl;

R⁵⁰* is C₁₋₆alkyl;

W in each occurrence is independently selected from —N(R^{3a})—C(O)— and —N(R^{3a})—S(O)₂—; and

X in each occurrence is independently selected from C₂₋₆alkenylene and C₂₋₆alkynylene.

In another aspect, R³ in each occurrence is independently selected from X—R⁵, W—R⁶, —C(H)═N(R^{3y}), —C(H)═N—N(R^{3a})—C(O)—R^{3b}, —N═C(R^{3y})₂, —N(H)—S(O)₂—N(R^{3y})₂, and —N(H)—C(O)—N(R^{3y})₂;

R^{3b} is methyl;

R^{3y} in each occurrence is independently selected from H, cyclopentyl, t-butyl, ethyl, imidazolyl, isoxazolyl, methyl, morpholino, oxazolidinonyl, phenyl, pyrazolyl, pyridyl, pyrrolidinyl, thiazolyl, thienyl, and 4H-1,2,4-triazolyl, wherein said cyclopentyl, t-butyl, ethyl, imidazolyl, isoxazolyl, methyl, morpholino, oxazolidinonyl, phenyl, pyrazolyl, pyridyl, pyrrolidinyl, thiazolyl, thienyl, and 4H-1,2,4-triazolyl in each occurrence are optionally and independently substituted on carbon with one or more R³⁰, and wherein the nitrogen of any —NH— moiety of said imidazolyl, oxazolidinonyl, pyrazolyl, pyrrolidinyl, thiazolyl, and 4H-1,2,4-triazolyl in each occurrence are optionally and independently substituted with R³⁰*;

R⁵ in each occurrence is independently selected from pyridyl, imidazolyl, pyrazinyl, and —Si(R^{5b})₃, wherein the nitrogen of any —NH— moiety of said imidazolyl in each occurrence is optionally substituted with R⁵⁰*;

R^{5b} is methyl;

R⁶ in each occurrence is independently selected from morpholino, 2-oxoimidazolidinyl, piperidinyl, and pyrrolidinyl;

R³⁰ in each occurrence is independently selected from chloro, —CN, methyl, and —OR^{30a},

R^{30a} is methyl;

R³⁰* is methyl;

R⁵⁰* is methyl;

W in each occurrence is independently selected from —N(H)—C(O)— and —N(H)—S(O)₂—; and

X in each occurrence is independently selected from ethene-1,2-diyl and ethyne-1,2-diyl.

In still another aspect, R³ in each occurrence is independently selected from X—R⁵, W—R⁶, —C(H)═N—R^{3y}, —C(H)═N—N(R^{3a})—C(O)—R^{3b}, —N═C(H)(R⁴), —N(H)—S(O)₂—N(R^{3y})₂, and —N(H)—C(O)—N(H)(R^{3y});

R^{3b} is methyl;

R^{3y} in each occurrence is independently selected from 4-chloro-1H-pyrazol-3-yl, cyclopentyl, t-butyl, ethyl, imidazol-4-yl, 5-methylisoxazol-3-yl, 2-methoxyethyl, methyl, 1-methyl-1H-imidazol-2-yl, 1-methyl-1H-imidazol-5-yl, morpholino, 2-oxo-1,3-oxazolidin-3-yl, phenyl, 1-methyl-1H-pyrazol-4-yl, pyrazol-3-yl, 1,3-dimethyl-1H-pyrazol-5-yl, 1,4-dimethyl-1H-pyrazol-5-yl, pyridin-2-yl, pyridin-3-yl, pyridin-4-yl, pyrrolidinyl, thiazol-5-yl, thiazol-2-yl, 2-cyano5-methylthien-3-yl, and 3,5-dimethyl-4H-1,2,4-triazol-4-yl, and 4H-1,2,4-triazol-4-yl;

R⁵ in each occurrence is independently selected from pyridin-2-yl, pyridin-3-yl, pyridin-4-yl, 1-methyl-1H-imidazol-2-yl, pyrazin-2-yl, and —Si(Me)₃;

R⁶ in each occurrence is independently selected from piperidin-1-yl, morpholino, pyrrolidin-1-yl, and 2-2-oxoimidazolidin-1-yl;

W in each occurrence is independently selected from —N(H)—C(O)— and —N(H)—S(O)₂—; and

X in each occurrence is independently selected from ethene-1,2-diyl and ethyne-1,2-diyl.

In yet another aspect, R³ in each occurrence is X—R⁵;

R⁵ is —Si(R^{5b})₃;

R^{5b}is C₁₋₆alkyl; and

X is C₂₋₆alkynylene.

In a further aspect, R³ is (trimethylsilyl)ethynyl.

In still a further aspect, R³ in each occurrence is independently selected from —X—R⁵, —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R⁴)₂, —C(R^{3a})═N—N(R^{3a})—C(O)—N(R^{3y})₂, and —N(R^{3a})—C(O)—N(R^{3y})₂;

R^{3a} in each occurrence is independently selected from H and C₁₋₆alkyl;

R^{3b} in each occurrence is independently selected from C₁₋₆alkyl and carbocyclyl, wherein said C₁₋₆alkyl and carbocyclyl in each occurrence are optionally and independently substituted on carbon with one or more R³⁰;

R^{3y} in each occurrence is independently selected from H, C₁₋₆alkyl , carbocyclyl, and heterocyclyl, wherein said C₁₋₆alkyl, carbocyclyl, and heterocyclyl in each occurrence are optionally and independently substituted on carbon with one or more R³⁰;

R⁵ in each occurrence is independently selected from heterocyclyl and —Si(R^{5b})₃, wherein said heterocyclyl is optionally substituted on carbon with one or more R⁵⁰, and wherein if said heterocyclyl contains an —NH-moiety, that nitrogen in each occurrence is optionally and independently substituted with R^{50*};

R^{5b} is C₁₋₆alkyl;

R⁶ is non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and heterocyclyl;

R^{50*} is C₁₋₆alkyl;

R^{60*} in each occurrence is independently selected from C₁₋₆alkyl, heterocyclyl and —C(O)₂R^{60c};

R^{60c} is C₁₋₆alkyl;

W in each occurrence is independently selected from —N(R^{3a})—C(O)—, —C(O)—N(R^{3a})—, and —N(R^{3a})—S(O)₂—; and

X is C₂₋₆alkynylene.

In yet a further aspect, R³ in each occurrence is independently selected from —X—R⁵, —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})₂, and —N(R^{3a})—C(O)—N(R^{3y})₂;

R^{3a} in each occurrence is independently selected from H and C₁₋₆alkyl;

R^{3b} in each occurrence is independently selected from C₁₋₆alkyl and 3- to 6-membered carbocyclyl, wherein said C₁₋₆alkyl and 3- to 6-membered carbocyclyl in each occurrence are optionally and independently substituted on carbon with one or more R³⁰;

R^{3y} in each occurrence is independently selected from H, C₁₋₆alkyl, 3- to 6-membered carbocyclyl, and 5- or 6-membered heterocyclyl, wherein said C₁₋₆alkyl, 3- to 6-membered carbocyclyl, and 5- or 6-membered heterocyclyl in each occurrence are optionally and independently substituted on carbon with one or more R³⁰;

R⁵ in each occurrence is independently selected from 5- or 6-membered heterocyclyl and —Si(R^{5b})₃, wherein said 5- or 6-membered heterocyclyl is optionally substituted on carbon with one or more R⁵⁰, and wherein if said 5- or 6-membered heterocyclyl contains an —NH-moiety, that nitrogen in each occurrence is optionally and independently substituted with R^{50*};

R^{5b} is C₁₋₆alkyl;

R⁶ is 5 or 6-membered non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and 5- or 6-membered heterocyclyl;

R^{50*} is C₁₋₆alkyl;

R^{60*} in each occurrence is independently selected from C₁₋₆alkyl, 5- or 6-membered heterocyclyl, and —C(O)₂R^{60c};

R^{60c} is C₁₋₆alkyl;

W in each occurrence is independently selected from —N(R^{3a})—C(O)—, —C(O)—N(R^{3a})—, and —N(R^{3a})—S(O)₂—; and

X is C₂₋₆alkynylene.

In one aspect, R³ in each occurrence is independently selected from —X—R⁵, —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})₂, and —N(R^{3a})—C(O)—N(R^{3y})₂;

R^{3a} in each occurrence is independently selected from H and C₁₋₆alkyl;

R^{3b} in each occurrence is independently selected from C₁₋₆alkyl and 3- to 6-membered carbocyclyl, wherein said C₁₋₆alkyl in each occurrence is optionally and independently substituted on carbon with one or more R³⁰;

R^{3y} in each occurrence is independently selected from H, C₁₋₆alkyl, 3- to 6-membered carbocyclyl, and 5- or 6-membered heterocyclyl, wherein said C₁₋₆alkyl, 3- to 6-membered carbocyclyl, and 5- or 6-membered heterocyclyl in each occurrence are optionally and independently substituted on carbon with one or more R³⁰;

R⁵ in each occurrence is independently selected from 5- or 6-membered heteroaryl and —Si(R^{5b})₃, wherein said 5- or 6-membered heteroaryl is optionally substituted on carbon with one or more R⁵⁰, and wherein if said 5- or 6-membered heteroaryl contains an —NH-moiety, that nitrogen in each occurrence is optionally and independently substituted with R^{50*};

R^{5b} is C₁₋₆alkyl;

R⁶ is 5 or 6-membered non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and 5- or 6-membered heteroaryl;

R^{50*} is C₁₋₆alkyl;

R^{60*} in each occurrence is independently selected from C₁₋₆alkyl, 5- or 6-membered heteroaryl, and —C(O)₂R^{60c};

R^{60c} is C₁₋₆alkyl;

W in each occurrence is independently selected from —N(R^{3a})—C(O)—, —C(O)—N(R^{3a})—, and —N(R^{3a})—S(O)₂—; and

X is C₂₋₆alkynylene.

In another aspect, R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;

R^{3a} in each occurrence is independently selected from H and C₁₋₆alkyl;

R^{3b} in each occurrence is independently selected from C₁₋₆alkyl and 3- to 6-membered carbocyclyl, wherein said C₁₋₆alkyl in each occurrence is optionally and independently substituted on carbon with one or more R³⁰;

R^{3y} in each occurrence is independently selected from H, C₁₋₆alkyl, 3- to 6-membered carbocyclyl, and 5- or 6-membered heterocyclyl, wherein said C₁₋₆alkyl, 3- to 6-membered carbocyclyl, and 5- or 6-membered heterocyclyl in each occurrence are optionally and independently substituted on carbon with one or more R³⁰;

R⁴ in each occurrence is independently selected from H and halo;

R⁵ in each occurrence is independently selected from 5- or 6-membered heterocyclyl and —Si(R^{5b})₃, wherein said 5- or 6-membered heterocyclyl is optionally substituted on carbon with one or more R⁵⁰, and wherein if said 5- or 6-membered heterocyclyl contains an —NH-moiety, that nitrogen in each occurrence is optionally and independently substituted with R^{50*};

R^{5b} is C₁₋₆alkyl;

R⁶ is 5 or 6-membered non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and 5- or 6-membered heterocyclyl;

R^{50*} is C₁₋₆alkyl;

R^{60*} in each occurrence is independently selected from C₁₋₆alkyl, 5- or 6-membered heterocyclyl, and —C(O)₂R^{60c};

R^{60c} is C₁₋₆alkyl;

W in each occurrence is independently selected from —N(H)—C(O)—, —C(O)—N(H)—, and —N(H)—S(O)₂—; and

X is C₂₋₆alkynylene.

In still another aspect, R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;

R^{3a} in each occurrence is independently selected from H and methyl;

R^{3b} in each occurrence is independently selected from methyl, t-butyl, and cyclopropyl, wherein said methyl, t-butyl, and cyclopropyl in each occurrence are optionally and independently substituted on carbon with one or more R³⁰;

R^{3y} in each occurrence is independently selected from H, 2,4-dioxoimidazolidinyl, ethyl, methyl, morpholinyl, phenyl, pyrazinyl, pyridinyl, pyrimidinyl, pyrrolidinyl, and 4H-1,2,4-triazolyl, wherein said 2,4-dioxoimidazolidinyl, morpholinyl, phenyl, pyrazinyl, pyridinyl, pyrimidinyl, pyrrolidinyl, and 4H-1,2,4-triazolyl in each occurrence are optionally and independently substituted on carbon with one or more methyl;

R⁵ in each occurrence is independently selected from —Si(Me)₃, 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl, wherein said 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl are optionally substituted on carbon with one or more R⁵⁰, and wherein the —NH— nitrogen of said imidazolyl, in each occurrence is optionally and independently substituted with methyl;

R⁶ in each occurrence is independently selected from dioxidotetrahydrothiophenyl, morpholinyl, oxoimidazolidinyl, 2-oxotetrahydrofuranyl, piperidinyl, pyrrolidinyl, tetrahydropyranyl, and tetrahydropyranyl, wherein the —NH— nitrogen of said morpholinyl, oxoimidazolidinyl, piperidinyl, and pyrrolidinyl in each occurrence is optionally and independently substituted with R^{60*};

R³⁰ in each occurrence is independently selected from methyl, —CN, and methoxy;

R⁵⁰ in each occurrence is independently selected from methyl, tetrazolyl, and pyrazolyl;

R^{60*} in each occurrence is independently selected from methyl, pyridinyl, and —C(O)₂Me;

W in each occurrence is independently selected from —N(H)—C(O)—, —C(O)—N(H)—, and —N(H)—S(O)₂—; and

X is ethyne-1,2-diyl.

In yet another aspect, R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;

R^{3a} in each occurrence is independently selected from H and methyl;

R^{3b} in each occurrence is independently selected from methyl, t-butyl, and cyclopropyl, wherein said methyl, t-butyl and cyclopropyl in each occurrence are optionally and independently substituted on carbon with one or more R³⁰;

R^{3y} in each occurrence is independently selected from H, 2,4-dioxoimidazolidin-1-yl, ethyl, methyl, morpholin-4-yl, phenyl, pyrazin-2-yl, pyridin-2-yl, pyrimidin-2-yl, pyrrolidin-1-y, and 4H-1,2,4-triazol-4-yl, wherein said 2,4-dioxoimidazolidin-1-yl, morpholin-4-yl, phenyl, pyrazin-2-yl, pyridin-2-yl, pyrimidin-2-yl, pyrrolidin-1-yl, and 4H-1,2,4-triazol-4-yl in each occurrence are optionally and independently substituted on carbon with one or more methyl;

R⁵ in each occurrence is independently selected from —Si(Me)₃, 1,3-benzothiazol-2-yl, 1-benzothiophen-2-yl, 1,3-benzoxazol-2-yl, imidazol-2-yl, imidazol-4-yl, pyrazin-2-yl, pyridin-2-yl, pyridiny-3-yl, pyridin-4-yl, pyrimidin-2-yl, 1,3,4-thiadiazol-2-yl, thiazol-2-yl, thiazol-5-yl, and thiophen-2-yl, wherein said 1,3-benzothiazol-2-yl, 1-benzothiophen-2-yl, 1,3-benzoxazol-2-yl, imidazol-2-yl, imidazol-4-yl, pyrazin-2-yl, pyridin-2-yl, pyridiny-3-yl, pyridin-4-yl, pyrimidin-2-yl, 1,3,4-thiadiazol-2-yl, thiazol-2-yl, thiazol-5-yl, and thiophen-2-yl are optionally substituted on carbon with one or more R⁵⁰, and wherein the —NH— nitrogen of said imidazol-2-yl and imidazol-4-yl in each occurrence is optionally and independently substituted with methyl;

R⁶ in each occurrence is independently selected from dioxidotetrahydrothiophen-3-yl, morpholin-4-yl, oxoimidazolidin-1-yl, 2-oxotetrahydrofuran-3-yl, piperidin-3-y, piperidin-4-yl, pyrrolidin-3-yl, tetrahydrofuran-3-yl, tetrahydropyran-3-yl, tetrahydropyran-4-yl, wherein the —NH— nitrogen of said oxoimidazolidin-1-yl, piperidin-3-yl, piperidin-4-yl, and pyrrolidin-3-yl in each occurrence is optionally and independently substituted with R^{60*};

R³⁰ in each occurrence is independently selected from methyl, —CN, and methoxy;

R⁵⁰ in each occurrence is independently selected from methyl, tetrazolyland pyrazolyl;

R^{60*} in each occurrence is independently selected from methyl, pyridinyl and —C(O)₂Me;

W in each occurrence is independently selected from —N(H)—C(O)—, —C(O)—N(H)—, and —N(H)—S(O)₂—; and

X is ethyne-1,2-diyl.

In a further aspect, R³ is —X—R⁵;

R⁵ in each occurrence is independently selected from heterocyclyl and —Si(R^{5b})₃, wherein said heterocyclyl is optionally substituted on carbon with one or more R⁵⁰, and wherein if said heterocyclyl contains an —NH-moiety, that nitrogen in each occurrence is optionally and independently substituted with R^{50*};

R^{5b} is C₁₋₆alkyl;

R⁵⁰ in each occurrence is independently selected from C₁₋₆alkyl and heterocyclyl;

R^{50*} is C₁₋₆alkyl;

X is C₂₋₆alkynylene; and

n is 1.

In still a further aspect, R³ is —X—R⁵;

R⁵ in each occurrence is independently selected from 5- or 6-membered heterocyclyl and —Si(R^{5b})₃, wherein said 5- or 6-membered heterocyclyl is optionally substituted on carbon with one or more R⁵⁰, and wherein if said 5- or 6-membered heterocyclyl contains an —NH-moiety, that nitrogen in each occurrence is optionally and independently substituted with R^{50*};

R^{5b} is C₁₋₆alkyl;

R⁵⁰ in each occurrence is independently selected from C₁₋₆alkyl and 5- or 6-membered heterocyclyl;

R^{50*} is C₁₋₆alkyl; and

X is C₂₋₆alkynylene.

In yet a further aspect, R³ in each occurrence is independently selected from —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b} , —C(R^{3a})═N—N(R^{3y})₂, —C(R^{3a})═N—N(R^{3a})—C(O)—N(R^{3y})₂, and —N(R^{3a})—C(O)—N(R^{3y})₂;

R^{3a} in each occurrence is independently selected from H and C₁₋₆alkyl;

R^{3b} in each occurrence is independently selected from C₁₋₆alkyl and carbocyclyl, wherein said C₁₋₆alkyl and carbocyclyl in each occurrence are optionally and independently substituted on carbon with one or more R³⁰;

R^{3y} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰;

R⁶ is non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R^{60*} in each occurrence is independently selected from C₁₋₆alkyl, heterocyclyl and —C(O)₂R^{60c};

R^{60c} is C₁₋₆alkyl; and

W in each occurrence is independently selected from —N(R^{3a})—C(O)—, —C(O)—N(R^{3a})—, and —N(R^{3a})—S(O)₂—.

In one further aspect, R³ in each occurrence is independently selected from —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})₂, and —N(R^{3a})—C(O)—N(R^{3y})₂;

R^{3a} in each occurrence is independently selected from H and C₁₋₆alkyl;

R^{3b} in each occurrence is independently selected from C₁₋₆alkyl and 3- to 6-membered carbocyclyl, wherein said C₁₋₆alkyl and 3- to 6-membered carbocyclyl in each occurrence are optionally and independently substituted on carbon with one or more R³⁰;

R^{3y} in each occurrence is independently selected from H, C₁₋₆alkyl, 3- to 6-membered carbocyclyl, and 5- or 6-membered heterocyclyl, wherein said C₁₋₆alkyl, 3- to 6-membered carbocyclyl, and 5- or 6-membered heterocyclyl in each occurrence are optionally and independently substituted on carbon with one or more R³⁰;

R⁶ is 5 or 6-membered non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R^{60*} in each occurrence is independently selected from C₁₋₆alkyl, 5- or 6-membered heterocyclyl, and —C(O)₂R^{60c};

R^{60c} is C₁₋₆alkyl; and

W in each occurrence is independently selected from —N(R^{3a})—C(O)—, —C(O)—N(R^{3a})—, and —N(R^{3a})—S(O)₂—.

In another aspect, R³ is —W—R⁶;

R^{3a} in each occurrence is independently selected from H and C₁₋₆alkyl;

R⁶ is 5 or 6-membered non-aromatic heterocyclyl, wherein if said non-aromatic heterocyclyl contains an —NH— moiety, that nitrogen in each occurrence is optionally and independently substituted with R^{60*};

R^{60*} in each occurrence is independently selected from C₁₋₆alkyl, 5- or 6-membered heteroaryl, and —C(O)₂R^{60c};

R^{60c} is C₁₋₆alkyl; and

W in each occurrence is independently selected from —N(R^{3a})—C(O)—, —C(O)—N(R^{3a})—, and —N(R^{3a})—S(O)₂—.

R⁴

In one aspect, R⁴ is H.

In another aspect, R⁴ in each occurrence is independently selected from H and halo.

In still another aspect, R⁴ in each occurrence is independently selected from H and fluoro.

Ring A

In one aspect, Ring A is a 6-membered non-aromatic heterocyclic ring, wherein
1) said 6-membered heterocyclic ring optionally contains, in addition to the nitrogen, an —O— group; and
2) said 6-membered heterocyclic ring is optionally substituted on carbon with one or more R⁷; and

R⁷ is C_{1 -6} alkyl.

In another aspect, Ring A is a morpholine ring, wherein said morpholine ring is optionally substituted on carbon with one or more R⁷; and

R⁷ is C_{1 -6} alkyl.

In still another aspect, Ring A is a morpholine ring, wherein said morpholine ring is optionally substituted on carbon with one or more R⁷; and

R⁷ is methyl.

In yet another aspect, Ring A is a 2,6-dimethylmorpholine ring.

n

In one aspect, n is 1 or 2.

In another aspect, n is 1.

R³ and n

In one aspect, R³ is selected from X—R⁵, W—R⁶, —C(H)═N—R^{3y}, —C(H)═N—N(R^{3a})—C(O)—R^{3b}, —N═C(R^{3y})₂, —N(H)—S(O)₂—N(R^{3y})₂, and —N(H)—C(O)—NR(^{3y})₂;

R^{3b} is C₁₋₆alkyl;

R^{3y} in each occurrence is independently selected from H, C₁₋₆alkyl, carbocyclyl, and heterocyclyl, wherein said C₁₋₆alkyl, carbocyclyl, and heterocyclyl is optionally substituted on carbon with one or more R³⁰, and wherein if said heterocyclyl contains an —NH— moiety, that nitrogen in each occurrence is optionally and independently substituted with R³⁰*;

R^{3a} is H;

R⁵ is selected from heterocyclyl and —Si(R^{5b})₃, wherein if said heterocyclyl contains an —NH— moiety, that nitrogen in each occurrence is optionally and independently substituted with R⁵⁰*;

R^{5b} is C₁₋₆alkyl;

R⁶ is non-aromatic heterocyclyl;

R³⁰ in each occurrence is independently selected from halo, —CN, C₁₋₆alkyl, and —OR^{30a};

R^{30a} is C₁₋₆alkyl;

R³⁰* is C₁₋₆alkyl;

R⁵⁰* is C₁₋₆alkyl;

W is selected from —N(R^{3a})—C(O)— and —N(R^{3a})—S(O)₂—;

X is selected from C₂₋₆alkenylene and C₂₋₆alkynylene; and

n is 1.

In another aspect, R³ is selected from X—R⁵, W—R⁶, —C(H)═N(R^{3y}), —C(H)═N—N(R^{3a})—C(O)—R^{3b}, —N═C(R^{3y})₂—N(H)—S(O)₂—N(R^{3y})₂, and —N(H)—C(O)—N(R^{3y})₂;

R^{3b} is methyl;

R^{3y}in each occurrence is independently selected from H, cyclopentyl, t-butyl, ethyl, imidazolyl, isoxazolyl, methyl, morpholino, oxazolidinonyl, phenyl, pyrazolyl, pyridyl, pyrrolidinyl, thiazolyl, thienyl, and 4H-1,2,4-triazolyl, wherein said cyclopentyl, t-butyl, ethyl, imidazolyl, isoxazolyl, methyl, morpholino, oxazolidinonyl, phenyl, pyrazolyl, pyridyl, pyrrolidinyl, thiazolyl, thienyl, and 4H-1,2,4-triazolyl in each occurrence are optionally and independently substituted on carbon with one or more R³⁰, and wherein the nitrogen of any —NH— moiety of said imidazolyl, oxazolidinonyl, pyrazolyl, pyrrolidinyl, thiazolyl, and 4H-1,2,4-triazolyl in each occurrence are optionally and independently substituted with R³⁰*;

R⁵ is selected from pyridyl, imidazolyl, pyrazinyl, and —Si(R^{5b})₃, wherein the nitrogen of any —NH— moiety of said imidazolyl in each occurrence is optionally and independently substituted with R⁵⁰*;

R^{5b} is methyl;

R⁶ is selected from morpholino, 2-oxoimidazolidinyl, piperidinyl, pyrrolidinyl;

R³⁰ in each occurrence is independently selected from chloro, —CN, methyl, —OR^{30a},

R^{30a} in each occurrence is methyl;

R³⁰* in each occurrence is methyl;

R⁵⁰* in each occurrence is methyl;

W is selected from —N(H)—C(O)— and —N(H)—S(O)₂—;

X is selected from ethene-1,2-diyl and ethyne-1,2-diyl; and

n is 1.

In still another aspect, R³ is selected from X—R⁵, W—R⁶, —C(H)═N—R^{3y}, —C(H)═N—N(R^{3a})—C(O)—R^{3b}, —N═CH—R^{3y}, —N(H)—S(O)₂—N(R^{3y})₂, and —N(H)—C(O)—N(H)(R^{3y});

R^{3b} is methyl;

R^{3y} in each occurrence is independently selected from 4-chloro-1H-pyrazol-3-yl, cyclopentyl, t-butyl, ethyl, imidazol-4-yl, 5-methylisoxazol-3-yl, 2-methoxyethyl, methyl, 1-methyl-1H-imidazol-2-yl, 1-methyl-1H-imidazol-5-yl, morpholino, 2-oxo-1,3-oxazolidin-3-yl, phenyl, 1-methyl-1H-pyrazol-4-yl, pyrazol-3-yl, 1,3-dimethyl-1H-pyrazol-5-yl, 1,4-dimethyl-1H-pyrazol-5-yl, pyridin-2-yl, pyridin-3-yl, pyridin-4-yl, pyrrolidinyl, thiazol-5-yl, thiazol-2-yl, 2-cyano5-methylthien-3-yl, and 3,5-dimethyl-4H-1,2,4-triazol-4-yl, and 4H-1,2,4-triazol-4-yl;

R⁵ is selected from pyridin-2-yl, pyridin-3-yl, pyridin-4-yl, 1-methyl-1H-imidazol-2-yl, pyrazin-2-yl, and —Si(Me)₃;

R⁶ is selected from piperidin-1-yl, morpholino, pyrrolidin-1-yl, 2-2-oxoimidazolidin-1-yl;

W is selected from —N(H)—C(O)— and —N(H)—S(O)₂—;

X is selected from ethene-1,2-diyl and ethyne-1,2-diyl; and

n is 1.

In yet another aspect, R³ in each occurrence is X—R⁵;

R⁵ is and —Si(R^{5b})₃;

R^{5b} in each occurrence is C₁₋₆alkyl;

X is C₂₋₆alkynylene; and

n is 1.

In a further aspect, R³ in each occurrence is (trimethylsilyl)ethynyl; and

n is 1.

R⁴ and n

In one aspect, R⁴ is H; and

n is 1.

In another aspect, R⁴ is selected from H and halo; and

n is 1.

In still another aspect, R⁴ is selected from H and fluoro; and

n is 1.

R¹, R², R³, R⁴, Ring A, and n

In one aspect, R¹ is H;

R² is H;

R³ in each occurrence is independently selected from X—R⁵, W—R⁶, —C(H)═N—R^{3y}, —C(H)═N—N(R^{3a})—C(O)—R^{3b}, —N═C(R^{3y})₂, —N(H)—S(O)₂—N(R^{3y})₂, —N(H)—C(O)—NR(^{3y})₂;

R^{3b} is C₁₋₆alkyl;

R^{3y} in each occurrence is independently selected from H, C₁₋₆alkyl, carbocyclyl, and heterocyclyl, wherein said C₁₋₆alkyl, carbocyclyl, and heterocyclyl are optionally substituted on carbon with one or more R³⁰, and wherein if said heterocyclyl contains an —NH— moiety, that nitrogen in each occurrence is optionally and independently substituted with R³⁰*;

R^{3a} is H;

R⁴ is H;

R⁵ in each occurrence is independently selected from heterocyclyl and —Si(R^{5b})₃, wherein if said heterocyclyl contains an —NH— moiety, that nitrogen is optionally substituted with R⁵⁰*;

R^{5b} is C₁₋₆alkyl;

R⁶ is non-aromatic heterocyclyl;

R⁷ is C₁₋₆alkyl;

R³⁰ in each occurrence is independently selected from halo, —CN, C₁₋₆alkyl, and —OR^{30a};

R^{30a} is C₁₋₆alkyl;

R³⁰* is C₁₋₆alkyl;

R⁵⁰* is C₁₋₆alkyl;

W in each occurrence is independently selected from —N(R^{3a})—C(O)— and —N(R^{3a})—S(O)₂—;

X in each occurrence is independently selected from C₂₋₆alkenylene and C₂₋₆alkynylene;

Ring A is a 6-membered non-aromatic heterocyclic ring, wherein
1) said 6-membered heterocyclic ring optionally contains, in addition to the nitrogen to which Y is attached, an —O— group; and
2) said 6-membered heterocyclic ring is optionally substituted on carbon with one or more R⁷; and

n is 1 or 2.

In another aspect, R¹ is H;

R² is H;

R³ is selected from X—R⁵, W—R⁶, —C(H)═N—R^{3y}, —C(H)═N—N(R^{3a})—C(O)—R^{3b}, —N═C(R^{3y})₂, —N(H)—S(O)₂—N(R^{3y})₂, and —N(H)—C(O)—N(R^{3y})₂;

R^{3b} is methyl;

R^{3y} in each occurrence is independently selected from H, cyclopentyl, t-butyl, ethyl, imidazolyl, isoxazolyl, methyl, morpholino, oxazolidinonyl, phenyl, pyrazolyl, pyridyl, pyrrolidinyl, thiazolyl, thienyl, and 4H-1,2,4-triazolyl, wherein said cyclopentyl, t-butyl, ethyl, imidazolyl, isoxazolyl, methyl, morpholino, oxazolidinonyl, phenyl, pyrazolyl, pyridyl, pyrrolidinyl, thiazolyl, thienyl, and 4H-1,2,4-triazolyl in each occurrence are optionally and independently substituted on carbon with one or more R³⁰, and wherein the nitrogen of an —NH— moiety of said imidazolyl, oxazolidinonyl, pyrazolyl, pyrrolidinyl, thiazolyl, and 4H-1,2,4-triazolyl in each occurrence is optionally and independently substituted with R³⁰*;

R⁴ is H;

R⁵ is selected from pyridyl, imidazolyl, pyrazinyl, and —Si(R^{5b})₃, wherein the nitrogen of an —NH— moiety of said imidazolyl in each occurrence is optionally substituted with with R⁵⁰*;

R^{5b} is methyl;

R⁶ is selected from morpholino, 2-oxoimidazolidinyl, piperidinyl, and pyrrolidinyl;

R⁷ is C₁₋₆alkyl;

R³⁰ in each occurrence is independently selected from chloro, —CN, methyl, and —OR^{30a},

R^{30a} is methyl;

R³⁰* is methyl;

R⁵⁰* is methyl;

Ring A is a morpholine ring, wherein said morpholine ring is optionally substituted on carbon with one or more R⁷;

X is selected from ethene-1,2-diyl and ethyne-1,2-diyl;

W is selected from —N(H)—C(O)— and —N(H)—S(O)₂—; and

n is 1.

In still another aspect, R¹ is H;

R² is H;

R³ is selected from X—R⁵, W—R⁶, —C(H)═N—R^{3y}, —C(H)═N—N(R^{3a})—C(O)—R^{3b}, —N═C(H)(R^{3y}), —N(H)—S(O)₂—N(R^{3y})₂, and —N(H)—C(O)—N(H)(R^{3y});

R^{3b} is methyl;

R^{3y} in each occurrence is independently selected from 4-chloro-1H-pyrazol-3-yl, cyclopentyl, t-butyl, ethyl, imidazol-4-yl, 5-methylisoxazol-3-yl, 2-methoxyethyl, methyl, 1-methyl-1H-imidazol-2-yl, 1-methyl-1H-imidazol-5-yl, morpholino, 2-oxo-1,3-oxazolidin-3-yl, phenyl, 1-methyl-1H-pyrazol-4-yl, pyrazol-3-yl, 1,3-dimethyl-1H-pyrazol-5-yl, 1,4-dimethyl-1H-pyrazol-5-yl, pyridin-2-yl, pyridin-3-yl, pyridin-4-yl, pyrrolidinyl, thiazol-5-yl, thiazol-2-yl, 2-cyano5-methylthien-3-yl, and 3,5-dimethyl-4H-1,2,4-triazol-4-yl, 4H-1,2,4-triazol-4-yl;

R⁴ is H;

R⁵ is selected from pyridin-2-yl, pyridin-3-yl, pyridin-4-yl, 1-methyl-1H-imidazol-2-yl, pyrazin-2-yl, and —Si(Me)₃;

R⁶ is selected from piperidin-1-yl, morpholino, pyrrolidin-1-yl, and 2-2-oxoimidazolidin-1-yl;

W is selected from —N(H)—C(O)— and —N(H)—S(O)₂—;

X is selected from ethene-1,2-diyl and ethyne-1,2-diyl;

Ring A is a 2,6-dimethylmorpholine ring; and

n is 1.

In one aspect, the compound of Formula (I) may be a compound of Formula (II):

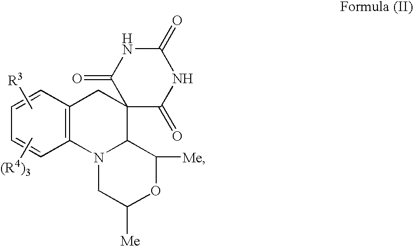

or a pharmaceutically acceptable salt thereof, wherein:

R³ in each occurrence is independently selected from —X—R⁵, —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R³³)₂, and —N(R^{3a})—C(O)—N(R^{3y})₂;

R^{3a} in each occurrence is independently selected from H and C₁₋₆alkyl;

R^{3b} in each occurrence is independently selected from C₁₋₆alkyl and carbocyclyl, wherein said C₁₋₆alkyl and carbocyclyl in each occurrence are optionally and independently substituted on carbon with one or more R³⁰;

R^{3y} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰;

R⁴ in each occurrence is independently selected from H and halo;

R⁵ in each occurrence is independently selected from heterocyclyl and —Si(R^{5b})₃, wherein said heterocyclyl is optionally substituted on carbon with one or more R⁵⁰, and wherein if said heterocyclyl contains an —NH-moiety, that nitrogen in each occurrence is optionally and independently substituted with R^{50*};

R^{5b} is C₁₋₆alkyl;

R⁶ is non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and heterocyclyl;

R^{50*} is C₁₋₆alkyl;

R^{60*} in each occurrence is independently selected from C₁₋₆alkyl, heterocyclyl and —C(O)₂R^{60c};

R^{60c} is C₁₋₆alkyl;

W in each occurrence is independently selected from —N(R^{3a})—C(O)—, —C(O)—N(R^{3a})—, and —N(R^{3a})—S(O)₂—; and

X is C₂₋₆alkynylene.

In another aspect, the compound of Formula (I) may be a compound of Formula (II):

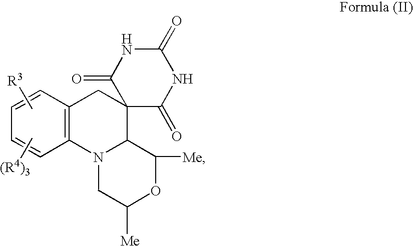

or a pharmaceutically acceptable salt thereof, wherein:

R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;

R^{3a} in each occurrence is independently selected from H and C₁₋₆alkyl;

R^{3b} in each occurrence is independently selected from C₁₋₆alkyl and 3- to 6-membered carbocyclyl, wherein said C₁₋₆alkyl in each occurrence is optionally and independently substituted on carbon with one or more R³⁰;

R^{3y} in each occurrence is independently selected from H, C₁₋₆alkyl, 3- to 6-membered carbocyclyl, and 5- or 6-membered heterocyclyl, wherein said C₁₋₆alkyl, 3- to 6-membered carbocyclyl, and 5- or 6-membered heterocyclyl in each occurrence are optionally and independently substituted on carbon with one or more R³⁰;

R⁴ in each occurrence is independently selected from H and halo;

R⁵ in each occurrence is independently selected from 5- or 6-membered heterocyclyl and —Si(R^{5b})₃, wherein said 5- or 6-membered heterocyclyl is optionally substituted on carbon with one or more R⁵⁰, and wherein if said 5- or 6-membered heterocyclyl contains an —NH-moiety, that nitrogen in each occurrence is optionally and independently substituted with R^{50*};

R^{5b} is C₁₋₆alkyl;

R⁶ is 5 or 6-membered non-aromatic heterocyclyl, wherein if said non-aromatic heterocyclyl contains an —NH— moiety, that nitrogen in each occurrence is optionally and independently substituted with R⁶⁰;

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and 5- or 6-membered heterocyclyl;

R^{50*} is C₁₋₆alkyl;

R^{60*} in each occurrence is independently selected from C₁₋₆alkyl, 5- or 6-membered heterocyclyl, and —C(O)₂R^{60c};

R^{60c} is C₁₋₆alkyl;

W in each occurrence is independently selected from —N(H)—C(O)—, —C(O)—N(H)—, and —N(H)—S(O)₂—; and

X is C₂₋₆alkynylene.

In still another aspect, the compound of Formula (I) may be a compound of Formula (II):

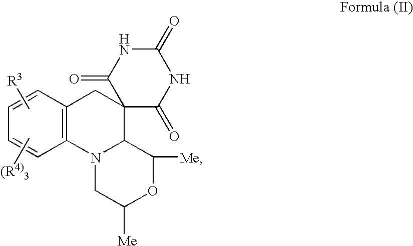

or a pharmaceutically acceptable salt thereof, wherein:

R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;

R^{3a} in each occurrence is independently selected from H and methyl;

R^{3b} in each occurrence is independently selected from methyl, t-butyl, and cyclopropyl, wherein said methyl, t-butyl, and cyclopropyl in each occurrence are optionally and independently substituted on carbon with one or more R³⁰;

R^{3y} in each occurrence is independently selected from H, 2,4-dioxoimidazolidinyl, ethyl, methyl, morpholinyl, phenyl, pyrazinyl, pyridinyl, pyrimidinyl, pyrrolidinyl, and 4H-1,2,4-triazolyl, wherein said 2,4-dioxoimidazolidinyl, morpholinyl, phenyl, pyrazinyl, pyridinyl, pyrimidinyl, pyrrolidinyl, and 4H-1,2,4-triazolyl in each occurrence are optionally and independently substituted on carbon with one or more methyl;

R⁴ in each occurrence is independently selected from H and fluoro;

R⁵ in each occurrence is independently selected from —Si(Me)₃, 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl, wherein said 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl are optionally substituted on carbon with one or more R⁵⁰, and wherein the —NH— nitrogen of said imidazolyl, in each occurrence is optionally and independently substituted with methyl;

R⁶ in each occurrence is independently selected from dioxidotetrahydrothiophenyl, morpholinyl, oxoimidazolidinyl, 2-oxotetrahydrofuranyl, piperidinyl, pyrrolidinyl, tetrahydropyranyl, and tetrahydropyranyl, wherein the —NH— nitrogen of said morpholinyl, oxoimidazolidinyl, piperidinyl, and pyrrolidinyl in each occurrence is optionally and independently substituted with R^{60*};

R³⁰ in each occurrence is independently selected from methyl —CN, and methoxy;

R⁵⁰ in each occurrence is independently selected from methyl, tetrazolyl and pyrazolyl;

R^{60*} in each occurrence is independently selected from methyl, pyridinyland —C(O)₂Me;

W in each occurrence is independently selected from —N(H)—C(O)—, —C(O)—N(H)—, and —N(H)—S(O)₂—; and

X is ethyne-1,2-diyl.

In one another aspect, the compound of Formula (I) may be a compound of Formula (III):

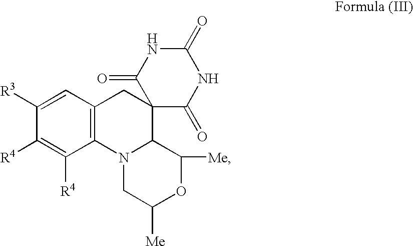

or a pharmaceutically acceptable salt thereof, wherein:

R³ in each occurrence is independently selected from —X—R⁵, —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})₂, and —N(R^{3a})—C(O)—N(R^{3y})₂;

R^{3a} in each occurrence is independently selected from H and C₁₋₆alkyl;

R^{3b} in each occurrence is independently selected from C₁₋₆alkyl and carbocyclyl, wherein said C₁₋₆alkyl and carbocyclyl in each occurrence are optionally and independently substituted on carbon with one or more R³⁰;

R^{3y} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰;

R⁴ in each occurrence is independently selected from H and halo;

R⁵ in each occurrence is independently selected from heterocyclyl and —Si(R^{5b})₃, wherein said heterocyclyl is optionally substituted on carbon with one or more R⁵⁰, and wherein if said heterocyclyl contains an —NH-moiety, that nitrogen in each occurrence is optionally and independently substituted with R^{50*};

R^{5b} is C₁₋₆alkyl;

R⁶ is non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and heterocyclyl;

R^{50*} is C₁₋₆alkyl;

R^{60*} in each occurrence is independently selected from C₁₋₆alkyl, heterocyclyl and —C(O)₂R^{60c};

R^{60c} is C₁₋₆alkyl;

W in each occurrence is independently selected from —N(R^{3a})—C(O)—, —C(O)—N(R^{3a})—, and —N(R^{3a})—S(O)₂—; and

X is C₂₋₆alkynylene.

In a further aspect, the compound of Formula (I) may be a compound of Formula (III):

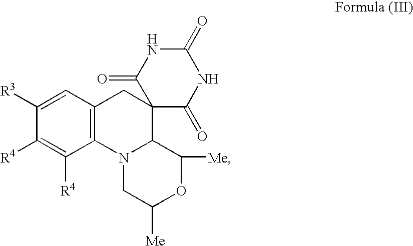

or a pharmaceutically acceptable salt thereof, wherein:

R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;

R^{3a} in each occurrence is independently selected from H and C₁₋₆alkyl;

R^{3b} in each occurrence is independently selected from C₁₋₆alkyl and 3- to 6-membered carbocyclyl, wherein said C₁₋₆alkyl in each occurrence is optionally and independently substituted on carbon with one or more R³⁰;

R^{3y} in each occurrence is independently selected from H, C₁₋₆alkyl, 3- to 6-membered carbocyclyl, and 5- or 6-membered heterocyclyl, wherein said C₁₋₆alkyl, 3- to 6-membered carbocyclyl, and 5- or 6-membered heterocyclyl in each occurrence are optionally and independently substituted on carbon with one or more R³⁰;

R⁴ in each occurrence is independently selected from H and halo;

R⁵ in each occurrence is independently selected from 5- or 6-membered heterocyclyl and —Si(R^{5b})₃, wherein said 5- or 6-membered heterocyclyl is optionally substituted on carbon with one or more R⁵⁰, and wherein if said 5- or 6-membered heterocyclyl contains an —NH-moiety, that nitrogen in each occurrence is optionally and independently substituted with R^{50*};

R^{5b} is C₁₋₆alkyl;

R⁶ is 5 or 6-membered non-aromatic heterocyclyl, wherein if said non-aromatic heterocyclyl contains an —NH— moiety, that nitrogen in each occurrence is optionally and independently substituted with R^{60*};

R³⁰ in each occurrence is independently selected from —CN, C₁₋₆alkyl, and —OR^{30a};

R^{30a} is C₁₋₆alkyl;

R⁵⁰ in each occurrence is independently selected from C₁₋₆alkyl and 5- or 6-membered heterocyclyl;

R^{50*} is C₁₋₆alkyl;

R^{60*} in each occurrence is independently selected from C₁₋₆alkyl, 5- or 6-membered heterocyclyl, and —C(O)₂R^{60c};

R^{60c} is C₁₋₆alkyl;

W in each occurrence is independently selected from —N(H)—C(O)—, —C(O)—N(H)—, and —N(H)—S(O)₂—; and

X is C₂₋₆alkynylene.

In still a further aspect, the compound of Formula (I) may be a compound of Formula (III):

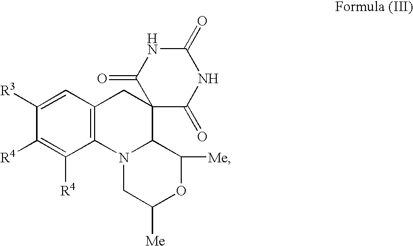

or a pharmaceutically acceptable salt thereof, wherein:

R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;

R^{3a} in each occurrence is independently selected from H and methyl;

R^{3b} in each occurrence is independently selected from methyl, t-butyl, and cyclopropyl, wherein said methyl, t-butyl, and cyclopropyl in each occurrence are optionally and independently substituted on carbon with one or more R³⁰;

R^{3y} in each occurrence is independently selected from H, 2,4-dioxoimidazolidinyl, ethyl, methyl, morpholinyl, phenyl, pyrazinyl, pyridinyl, pyrimidinyl, pyrrolidinyl, and 4H-1,2,4-triazolyl, wherein said 2,4-dioxoimidazolidinyl, morpholinyl, phenyl, pyrazinyl, pyridinyl, pyrimidinyl, pyrrolidinyl, and 4H-1,2,4-triazolyl in each occurrence are optionally and independently substituted on carbon with one or more methyl;

R⁴ in each occurrence is independently selected from H and fluoro;

R⁵ in each occurrence is independently selected from —Si(Me)₃, 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl, wherein said 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl are optionally substituted on carbon with one or more R⁵⁰, and wherein the —NH— nitrogen of said imidazolyl, in each occurrence is optionally and independently substituted with methyl;

R⁶ in each occurrence is independently selected from dioxidotetrahydrothiophenyl, morpholinyl, oxoimidazolidinyl, 2-oxotetrahydrofuranyl, piperidinyl, pyrrolidinyl, tetrahydropyranyl, and tetrahydropyranyl, wherein the —NH— nitrogen of said morpholinyl, oxoimidazolidinyl, piperidinyl, and pyrrolidinyl in each occurrence is optionally and independently substituted with R^{60*};

R³⁰ in each occurrence is independently selected from methyl, —CN, and methoxy;

R⁵⁰ in each occurrence is independently selected from methyl, tetrazolyl, and pyrazolyl;

R^{60*} in each occurrence is independently selected from methyl, pyridinyl, and —C(O)₂Me;

W in each occurrence is independently selected from —N(H)—C(O)—, —C(O)—N(H)—, and —N(H)—S(O)₂—; and

X is ethyne-1,2-diyl.

In yet a further aspect, the present invention provides a compound of Formula (I), or a pharmaceutically acceptable salt thereof, illustrated by the Examples, each of which provides a further independent aspect of the invention.

Typical compounds of Formula (I) are believed to inhibit bacterial DNA gyrase and are therefore of interest for their antibacterial effects. The inventive compounds are believed to be active against a variety of bacterial organisms, including both Gram positive and Gram negative aerobic and anaerobic bacteria.

These properties may be assessed, for example, using the testing methods shown below.

Bacterial Susceptibility Testing Methods

Compounds may be tested for antimicrobial activity by susceptibility testing in liquid media in a 96 well format. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension may be a 0.5 McFarland and a further 1 in 10 dilution may be advantageously made into the same liquid medium to prepare the final organism suspension in 100 μL. Plates may be incubated under appropriate conditions at 37° C. for 24 hours prior to reading. The Minimum Inhibitory Concentration (MIC) is intended to refer to the lowest drug concentration able to reduce growth by 80% or more.

Compounds may be evaluated against organisms such as Gram-positive species, including Staphylococcus aureus, Streptococcus pneumoniae, and Streptococcus pyogenes; and Gram-negative species including Haemophilus influenzae, and Moraxella catarrhalis. Compounds of the present invention are believed to have MIC's less than or equal to 8 μg/ml versus one or more of the organisms named above.

Representative bacterial DNA gyrase inhibition by compounds of the instant invention is indicated by in Table 1 below, which shows the minimum inhibitory concentration the compound of Example 1 has against the indicated bacteria.

TABLE 1

| Bacteria | MIC (μg/mL) |
|---|---|
| Sau516 | 8 |
| Sau517 | 8 |
| Spy838 | 16 |
| Spn548 | 16 |
| Spn538 | 32 |
| Hin446 | 4 |
| Hin737 | 16 |
| Hin158 | 0.13 |
| Mca445 | 2 |

DNA Gyrase Supercoiling Activity Fluorescence Polarisation Assay

In a black, 384-well polystyrene assay plate, 30 microliters/well of 5 nM Escherichia coli DNA gyrase A/B tetramer and 130 micrograms/ml of topologically relaxed plasmid containing the triplex-forming sequence TTCTTCTTCTTCTTCTTCTTCTTCTTC in an assay buffer consisting of 35 mM Tris-HCl (pH 7.5), 24 mM KCl, 4 mM MgCl₂, 2 mM dithiothreitol, 1.8 mM spermidine, 5% (v/v) glycerol, 200 nM bovine serum albumin, 0.8% dimethylsulfoxide, and 0.3 mM ATP may be incubated at ambient temperature for (typically 30 minutes) in the absence or presence of 5-10 different concentrations of test compound. The supercoiling reactions may be quenched by the addition of 10 microliters/well of 40 nM oligodeoxynucleotide probe in 3× triplex-forming buffer consisting of 150 mM NaCl, and 150 mM sodium acetate at pH 3.5. The oligodeoxynucleotide probe may be 5′-BODIPY-FL-labeled TTCTTCTTC. After 60 minutes, the fluorescence anisotropy of the BODIPY-FL may be measured in a Tecan Ultra plate reader, using 485 nm excitation and 535 nm emission filters equipped with polarizers. The IC₅₀ may be determined by nonliner regression using two control reactions. The first contains no test compound but 0.8% DMSO (100% activity) while the second control reaction contains 5 uM Ciprofloxacin and 0.8% DMSO (0% activity).

When tested in an in-vitro assay based on the DNA gyrase supercoiling activity fluorescence polarisation assay described above, the E. coli DNA gyrase supercoiling IC₅₀ assay inhibitory activity of the following Examples was measured at the indicated IC₅₀. A dash indicates that an IC₅₀ was not provided for that particular compound.

### Examples 1 to 10

| Example | IC₅₀ (μM) |
|---|---|
| 1 | 7.57 |
| 2 | 2.09 |
| 3 | 2.48 |
| 4 | 0.63 |
| 5 | 1.88 |
| 6 | 3.27 |
| 7 | 0.41 |
| 8 | 1.29 |
| 9 | 0.70 |
| 10 | 1.45 |

### Examples 11 to 20

| Example | IC₅₀ (μM) |
|---|---|
| 11 | 5.49 |
| 12 | 0.69 |
| 13 | 2.17 |
| 14 | 1.12 |
| 15 | 0.70 |
| 16 | 0.69 |
| 17 | 1.84 |
| 18 | 0.50 |
| 19 | 0.52 |
| 20 | 0.49 |

### Examples 21 to 30

| Example | IC₅₀ (μM) |
|---|---|
| 21 | 2.25 |
| 22 | 1.57 |
| 23 | 1.03 |
| 24 | 0.35 |
| 25 | 0.87 |
| 26 | 1.08 |
| 27 | 0.83 |
| 28 | 1.11 |
| 29 | 20.00 |
| 30 | 1.15 |

### Examples 31 to 40

| Example | IC₅₀ (μM) |
|---|---|
| 31 | 0.98 |
| 32 | 2.24 |
| 33 | 1.18 |
| 34 | 2.95 |
| 35 | 1.48 |
| 36 | 0.71 |
| 37 | 0.94 |
| 38 | 0.76 |
| 39 | 0.87 |
| 40 | 1.14 |

### Examples 41 to 50

| Example | IC₅₀ (μM) |
|---|---|
| 41 | 7.16 |
| 42 | 0.64 |
| 43 | 1.47 |
| 44 | 0.22 |
| 45 | — |
| 46 | — |
| 47 | 3.67 |
| 48 | 1.30 |
| 49 | 0.45 |
| 50 | 2.36 |

### Examples 51 to 60

| Example | IC₅₀ (μM) |
|---|---|
| 51 | 5.50 |
| 52 | 12.80 |
| 53 | 1.64 |
| 54 | 2.30 |
| 55 | 7.59 |
| 56 | 1.87 |
| 57 | 0.85 |
| 58 | 0.98 |
| 59 | 1.64 |
| 60 | 0.26 |

### Examples 61 to 70

| Example | IC₅₀ (μM) |
|---|---|
| 61 | 4.84 |
| 62 | 7.10 |
| 63 | 6.40 |
| 64 | 5.05 |
| 65 | >10 |
| 66 | 5.86 |
| 67 | 12.00 |
| 68 | 4.15 |
| 69 | 8.68 |
| 70 | 2.91 |

### Examples 71 to 80

| Example | IC₅₀ (μM) |
|---|---|
| 71 | 5.97 |
| 72 | 10.40 |
| 73 | 5.02 |
| 74 | 3.51 |
| 75 | 0.66 |
| 76 | 16.50 |
| 77 | 21.70 |
| 78 | 8.65 |
| 79 | 0.40 |
| 80 | — |

In one aspect there is provided a compound of Formula (I), or a pharmaceutically acceptable salt thereof, for use as a medicament.

In one aspect, the terms “infection” and “bacterial infection” may refer to a bacterial infection caused by Acinetobacter baumanii. In another aspect, the terms “infection” and “bacterial infection” may refer to a bacterial infection caused by Aeromonas hydrophile. In still another aspect, the terms “infection” and “bacterial infection” may refer to a bacterial infection caused by Bacillus anthracis. In yet another aspect, the terms “infection” and “bacterial infection” may refer to a bacterial infection caused by Bacteroides fragilis. In a further aspect, the terms “infection” and “bacterial infection” may refer to a bacterial infection caused by Bordatella pertussis. In still a further aspect, the terms “infection” and “bacterial infection” may refer to a bacterial infection caused by Burkholderia cepacia. In yet a further aspect, the terms “infection” and “bacterial infection” may refer to a bacterial infection caused by Chlamyida pneumoniae. In one aspect, the terms “infection” and “bacterial infection” may refer to a bacterial infection caused by Citrobacter freundii. In another aspect, the terms “infection” and “bacterial infection” may refer to a bacterial infection caused by Clostridium difficile. In still another aspect, the terms “infection” and “bacterial infection” may refer to a bacterial infection caused by Enterobacter cloacae. In yet another aspect, the terms “infection” and “bacterial infection” may refer to a bacterial infection caused by Enterococcus faecalis. In a further aspect, the terms “infection” and “bacterial infection” may refer to a bacterial infection caused by Enterococcus faecium. In still a further aspect, the terms “infection” and “bacterial infection” may refer to a bacterial infection caused by Enterobacter aerogenes. In yet a further aspect, the terms “infection” and “bacterial infection” may refer to a bacterial infection caused by Escherichia coli. In one aspect, the terms “infection” and “bacterial infection” may refer to a bacterial infection caused by Fusobacterium necrophorum. In another aspect, the terms “infection” and “bacterial infection” may refer to a bacterial infection caused by Haemophilus influenzae. In still another aspect, the terms “infection” and “bacterial infection” may refer to a bacterial infection caused by Haemophilus parainfluenzae. In yet another aspect, the terms “infection” and “bacterial infection” may refer to a bacterial infection caused by Haemophilus somnus. In a further aspect, the terms “infection” and “bacterial infection” may refer to a bacterial infection caused by Klebsiella oxytoca. In still a further aspect, the terms “infection” and “bacterial infection” may refer to a bacterial infection caused by Klebsiella pneumoniae. In yet a further aspect, the terms “infection” and “bacterial infection” may refer to a bacterial infection caused by Legionella pneumophila. In one aspect, the terms “infection” and “bacterial infection” may refer to a bacterial infection caused by Listeria monocytogenes. In another aspect, the terms “infection” and “bacterial infection” may refer to a bacterial infection caused by Moraxella catarrhalis. In still another aspect, the terms “infection” and “bacterial infection” may refer to a bacterial infection caused by Morganella morganii. In yet another aspect, the terms “infection” and “bacterial infection” may refer to a bacterial infection caused by Mycoplasma pneumoniae. In a further aspect, the terms “infection” and “bacterial infection” may refer to a bacterial infection caused by Neisseria gonorrhoeae. In still a further aspect, the terms “infection” and “bacterial infection” may refer to a bacterial infection caused by Neisseria meningitidis. In yet a further aspect, the terms “infection” and “bacterial infection” may refer to a bacterial infection caused by Pasteurella multocida. In one aspect, the terms “infection” and “bacterial infection” may refer to a bacterial infection caused by Proteus mirabilis. In another aspect, the terms “infection” and “bacterial infection” may refer to a bacterial infection caused by Proteus vulgaris. In still another aspect, the terms “infection” and “bacterial infection” may refer to a bacterial infection caused by Pseudomonas aeruginosa. In yet another aspect, the terms “infection” and “bacterial infection” may refer to a bacterial infection caused by Salmonella typhi. In a further aspect, the terms “infection” and “bacterial infection” may refer to a bacterial infection caused by Salmonella typhimurium. In still a further aspect, the terms “infection” and “bacterial infection” may refer to a bacterial infection caused by Serratia marcesens. In yet a further aspect, the terms “infection” and “bacterial infection” may refer to a bacterial infection caused by Shigella flexneria. In one aspect, the terms “infection” and “bacterial infection” may refer to a bacterial infection caused by Shigella dysenteriae. In another aspect, the terms “infection” and “bacterial infection” may refer to a bacterial infection caused by Staphylococcus aureus. In still another aspect, the terms “infection” and “bacterial infection” may refer to a bacterial infection caused by Staphylococcus epidermidis. In yet another aspect, the terms “infection” and “bacterial infection” may refer to a bacterial infection caused by Staphylococcus haemolyticus. In a further aspect, the terms “infection” and “bacterial infection” may refer to a bacterial infection caused by Staphylococcus intermedius. In still a further aspect, the terms “infection” and “bacterial infection” may refer to a bacterial infection caused by Staphylococcus saprophyticus. In yet a further aspect, the terms “infection” and “bacterial infection” may refer to a bacterial infection caused by Stenotrophomonas maltophila. In one aspect, the terms “infection” and “bacterial infection” may refer to a bacterial infection caused by Streptococcus agalactiae. In another aspect, the terms “infection” and “bacterial infection” may refer to a bacterial infection caused by Streptococcus mutans. In a still another aspect, the terms “infection” and “bacterial infection” may refer to a bacterial infection caused by Streptococcus pneumoniae. In yet another aspect, the terms “infection” and “bacterial infection” may refer to a bacterial infection caused by Streptococcus pyrogenes.

In one aspect, the terms “infection and “bacterial infection” may refer to a bacterial infection caused by a bacteria of the genus Aeromonas. In another aspect, the terms “infection and “bacterial infection” may refer to a bacterial infection caused by a bacteria of the genus Acinetobacter. In still another aspect, the terms “infection and “bacterial infection” may refer to a bacterial infection caused by a bacteria of the genus Bacillus. In yet another aspect, the terms “infection and “bacterial infection” may refer to a bacterial infection caused by a bacteria of the genus Bacteroides. In a further aspect, the terms “infection and “bacterial infection” may refer to a bacterial infection caused by a bacteria of the genus Bordetella. In still a further aspect, the terms “infection and “bacterial infection” may refer to a bacterial infection caused by a bacteria of the genus Burkholderia. In yet a further aspect, the terms “infection and “bacterial infection” may refer to a bacterial infection caused by a bacteria of the genus Chlamydophila. In one aspect, the terms “infection and “bacterial infection” may refer to a bacterial infection caused by a bacteria of the genus Citrobacter. In another aspect, the terms “infection and “bacterial infection” may refer to a bacterial infection caused by a bacteria of the genus Clostridium. In still another aspect, the terms “infection and “bacterial infection” may refer to a bacterial infection caused by a bacteria of the genus Enterobacter. In yet another aspect, the terms “infection and “bacterial infection” may refer to a bacterial infection caused by a bacteria of the genus Enterococcus. In a further aspect, the terms “infection and “bacterial infection” may refer to a bacterial infection caused by a bacteria of the genus Escherichia. In still a further aspect, the terms “infection and “bacterial infection” may refer to a bacterial infection caused by a bacteria of the genus Flavobacterium. In yet a further aspect, the terms “infection and “bacterial infection” may refer to a bacterial infection caused by a bacteria of the genus Fusobacterium. In one aspect, the terms “infection and “bacterial infection” may refer to a bacterial infection caused by a bacteria of the genus Haemophilus. In one aspect, the terms “infection and “bacterial infection” may refer to a bacterial infection caused by a bacteria of the genus Klebsiella. In another aspect, the terms “infection and “bacterial infection” may refer to a bacterial infection caused by a bacteria of the genus Legionella. In still another aspect, the terms “infection and “bacterial infection” may refer to a bacterial infection caused by a bacteria of the genus Listeria. In yet another aspect, the terms “infection and “bacterial infection” may refer to a bacterial infection caused by a bacteria of the genus Morganella. In a further aspect, the terms “infection and “bacterial infection” may refer to a bacterial infection caused by a bacteria of the genus Moraxella. In still a further aspect, the terms “infection and “bacterial infection” may refer to a bacterial infection caused by a bacteria of the genus Mycoplasma. In yet a further aspect, the terms “infection and “bacterial infection” may refer to a bacterial infection caused by a bacteria of the genus Neisseria. In one aspect, the terms “infection and “bacterial infection” may refer to a bacterial infection caused by a bacteria of the genus Pasteurella. In another aspect, the terms “infection and “bacterial infection” may refer to a bacterial infection caused by a bacteria of the genus Peptococci. In still another aspect, the terms “infection and “bacterial infection” may refer to a bacterial infection caused by a bacteria of the genus Peptostreptococci. In yet another aspect, the terms “infection and “bacterial infection” may refer to a bacterial infection caused by a bacteria of the genus Prevotella. In a further aspect, the terms “infection and “bacterial infection” may refer to a bacterial infection caused by a bacteria of the genus Proteus. In still a further aspect, the terms “infection and “bacterial infection” may refer to a bacterial infection caused by a bacteria of the genus Pseudomonas. In still another aspect, the terms “infection and “bacterial infection” may refer to a bacterial infection caused by a bacteria of the genus Salmonella. In yet a further aspect, the terms “infection and “bacterial infection” may refer to a bacterial infection caused by a bacteria of the genus Serratia. In one aspect, the terms “infection and “bacterial infection” may refer to a bacterial infection caused by a bacteria of the genus Shigella. In yet another aspect, the terms “infection and “bacterial infection” may refer to a bacterial infection caused by a bacteria of the genus Staphylococcus. In another aspect, the terms “infection and “bacterial infection” may refer to a bacterial infection caused by a bacteria of the genus Stenotrophomonas. In still another aspect, the terms “infection and “bacterial infection” may refer to a bacterial infection caused by a bacteria of the genus Streptococcus.

In one aspect, the terms “infection” and “bacterial infection” may refer to a gynecological infection. In another aspect the terms “infection” and “bacterial infection” may refer to a respiratory tract infection (RTI). In still another, the terms “infection” and “bacterial infection” may refer to a sexually transmitted disease. In yet another aspect, the terms “infection” and “bacterial infection” may refer to a urinary tract infection. In a further aspect, the terms “infection” and “bacterial infection” may refer to acute exacerbation of chronic bronchitis (ACEB). In yet a further aspect, the terms “infection” and “bacterial infection” may refer to acute otitis media. In one aspect, the terms “infection” and “bacterial infection” may refer to acute sinusitis. In another aspect, the terms “infection” and “bacterial infection” may refer to an infection caused by drug resistant bacteria. In still another aspect, the terms “infection” and “bacterial infection” may refer to catheter-related sepsis. In yet another aspect, the terms “infection” and “bacterial infection” may refer to chancroid. In a further aspect, the terms “infection” and “bacterial infection” may refer to chlamydia. In still a further aspect, the terms “infection” and “bacterial infection” may refer to community-acquired pneumonia (CAP). In yet a further aspect, the terms “infection” and “bacterial infection” may refer to complicated skin and skin structure infection. In one aspect, the terms “infection” and “bacterial infection” may refer to uncomplicated skin and skin structure infection. In another aspect, the terms “infection” and “bacterial infection” may refer to endocarditis. In still another aspect, the terms “infection” and “bacterial infection” may refer to febrile neutropenia. In yet another aspect, the terms “infection” and “bacterial infection” may refer to gonococcal cervicitis. In a further aspect, the terms “infection” and “bacterial infection” may refer to gonococcal urethritis. In still a further aspect, the terms “infection” and “bacterial infection” may refer to hospital-acquired pneumonia (HAP). In yet another aspect, the terms “infection” and “bacterial infection” may refer to osteomyelitis. In a further aspect, the terms “infection” and “bacterial infection” may refer to sepsis. In still a further aspect, the terms “infection” and “bacterial infection” may refer to syphilis.

In one aspect, there is provided the use of a compound of Formula (I), or a pharmaceutically acceptable salt thereof, in the manufacture of a medicament for the production of a bacterial DNA gyrase inhibitory effect, in a warm-blooded animal such as man.

In another aspect, there is provided the use a compound of Formula (I), or a pharmaceutically acceptable salt thereof, in the manufacture of a medicament for the treatment of a bacterial infection in a warm-blooded animal such as man.

In still another aspect, there is provided the use of a compound of Formula (I), or a pharmaceutically acceptable salt thereof, in the manufacture of a medicament for the treatment of urinary tract infections, pneumonia, prostatitis, skin and soft tissue infections, and intra-abdominal infections, in a warm-blooded animal such as man.

In yet another aspect, there is provided a method for producing a bacterial DNA gyrase inhibitory effect in a warm-blooded animal such as man, said method comprising administering to said animal an effective amount of a compound of Formula (I), or a pharmaceutically acceptable salt thereof.

In a further aspect, there is provided a method for treating a bacterial infection in a warm-blooded animal such as man, said method comprising administering to said animal an effective amount of a compound of formula (I), or a pharmaceutically acceptable salt thereof.

In still a further aspect, there is provided a method for treating urinary tract infections, pneumonia, prostatitis, skin and soft tissue infections, and intra-abdominal infections, in a warm-blooded animal such as man, said method comprising administering to said animal an effective amount of a compound of Formula (I), or a pharmaceutically acceptable salt thereof.

In yet a further aspect, there is provided a compound of Formula (I), or a pharmaceutically acceptable salt thereof, for use in producing a bacterial DNA gyrase inhibitory effect in a warm-blooded animal such as man.

In one aspect, there is provided a compound of Formula (I), or a pharmaceutically acceptable salt thereof, for use in treating a bacterial infection in a warm-blooded animal, such as man.

In another aspect, there is provided a compound of Formula (I), or a pharmaceutically acceptable salt thereof, for use in treating urinary tract infections, pneumonia, prostatitis, skin and soft tissue infections, and intra-abdominal infections, in a warm-blooded animal such as man.

In still another aspect, there is provided a pharmaceutical composition comprising a compound of Formula (I), or a pharmaceutically acceptable salt thereof, and at least one pharmaceutically acceptable carrier, diluent, or excipient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); coloring agents; flavoring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention, the pharmaceutical composition of this invention may also contain or be co-administered (simultaneously, sequentially or separately) with one or more known drugs selected from other clinically useful classes of antibacterial agents (for example, macrolides, quinolones, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

In addition to its use in therapeutic medicine, the compound of Formulas (I) and its pharmaceutically acceptable salts are also useful as pharmacological tools in the development and standardization of in vitro and in vivo test systems for the evaluation of the effects of inhibitors of DNA gyrase in laboratory animals such as cats, dogs, rabbits, monkeys, rats and mice, as part of the search for new therapeutic agents.

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5^{th} Edition, by Jerry March and Michael Smith, published by John Wiley & Sons 2001, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991) and as described hereinabove.

Compounds of Formula (I) may be prepared in a variety of ways. In one aspect, compounds of Formula (I) may be prepared according to the procedures describedin U.S. Pat. No. 7,208,490 to Pharmacia and Upjohn Company LLC, of which column 17, line 22 to column 84, line 22 is hereby incorporated by reference. Processes A through F shown below illustrate some methods for synthesizing compounds of Formula (I) (wherein Ring A, R¹, R², R³, R^{3a}, R^{3y}, R⁴, R⁵, R⁶, and n, unless otherwise defined, are as defined hereinabove). The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used. The Schemes and Processes are not intended to present an exhaustive list of methods for preparing the compounds of Formula (I); rather, additional techniques of which the skilled chemist is aware may be also be used for the compounds' synthesis. The claims are not intended to be limited to the structures shown in the Schemes and Processes.

The skilled chemist will be able to use and adapt the information contained and referenced within the above references, and accompanying Examples therein and also the Examples and Schemes herein, to obtain necessary starting materials and products.

In one aspect, compounds of Formula (I), or pharmaceutically acceptable salts thereof, may be prepared by:

Process A—reacting a compound of Formula (A1):

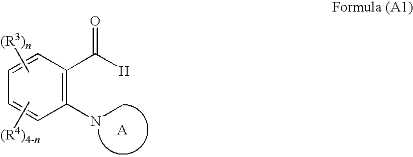

with a compound of Formula (A2):

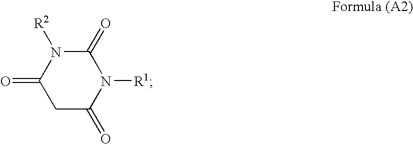

and thereafter if necessary:
i) converting a compound of Formula (I) into another compound of Formula (I);
ii) removing any protecting groups; and/or
iii) forming a pharmaceutically acceptable salt.

The reaction of Process A may be performed in a solvent such as methanol or butanol. The reaction may be advantageously performed at reflux temperatures.

In another aspect, compounds of Formula (I) in which R³ is —N(R^{3a})C(O)N(H)(R^{3y}) may be prepared by:

Process B—reacting a compound of Formula (A3):

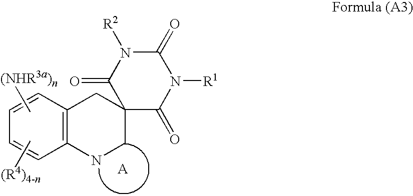

with a compound of Formula (A4):

R^{3y}—NCO

Formula (A4);

and thereafter if necessary:
i) converting a compound of Formula (I) into another compound of Formula (I);
ii) removing any protecting groups; and/or
iii) forming a pharmaceutically acceptable salt.

In still another aspect, compounds of Formula (I) in which R³ in each occurrence is independently selected from —N(R^{3a})C(O)R⁶, —N(R^{3a})C(O)N(R^{3y})₂, —N(R^{3a})S(O)₂R⁶, and —N(R^{3a})S(O)₂N(R^{3y})₂ may be prepared by:

Process C—reacting a compound of Formula (A3):

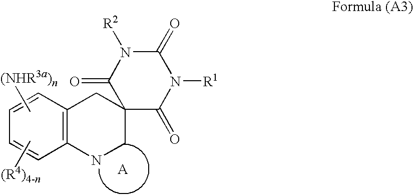

with a compound of Formula (A5):

R^{k}-Q-C1 Formula (A5);

and thereafter if necessary:
i) converting a compound of Formula (I) into another compound of Formula (I);
ii) removing any protecting groups; and/or
iii) forming a pharmaceutically acceptable salt,

wherein

Q in each occurrence is independently selected from —C(O)— and —S(O)₂—; and

R^{k} in each occurrence is independently selected from —N(R^{3y})₂ and non-aromatic heterocyclyl.

In yet another process, compounds of Formula (I) in which R³ is —N═C(H)(R^{3y}) may be prepared by:

Process D—reacting a compound of Formula (A3):

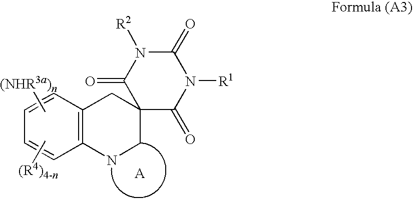

with a compound of Formula (A6):

R^{3y}—C(O)H Formula (A6);

and thereafter if necessary:
i) converting a compound of Formula (I) into another compound of Formula (I);
ii) removing any protecting groups; and/or
iii) forming a pharmaceutically acceptable salt.

In yet another process, compounds of Formula (I) in which R³ is —C₂alkynylene-R⁵ may be prepared by:

Process E—reacting a compound of Formula (A7):

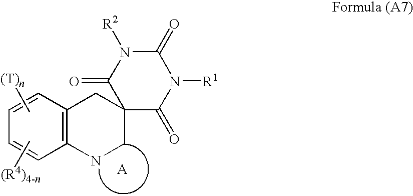

with a compound of Formula (A8):

≡—R⁵ Formula (A8);

under standard Sonogashira coupling conditions, and thereafter if necessary:
i) converting a compound of Formula (I) into another compound of Formula (I);
ii) removing any protecting groups; and/or
iii) forming a pharmaceutically acceptable salt,

wherein T is halo.

In yet another process, compounds of Formula (I) in which R³ is —C₂alkenylene-R⁵ may be prepared by:

Process F—reacting a compound of Formula (A7):

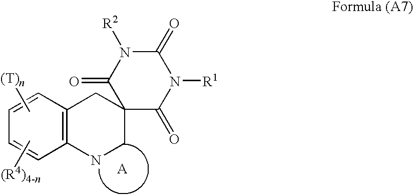

with a compound of Formula (A9):

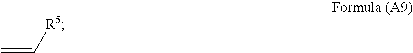

under standard Heck coupling conditions,

and thereafter if necessary:
i) converting a compound of Formula (I) into another compound of Formula (I);
ii) removing any protecting groups; and/or
iii) forming a pharmaceutically acceptable salt,

wherein T is halo.

Scheme 1 depicts a procedure by which compounds of Formula (A1) may be prepared.

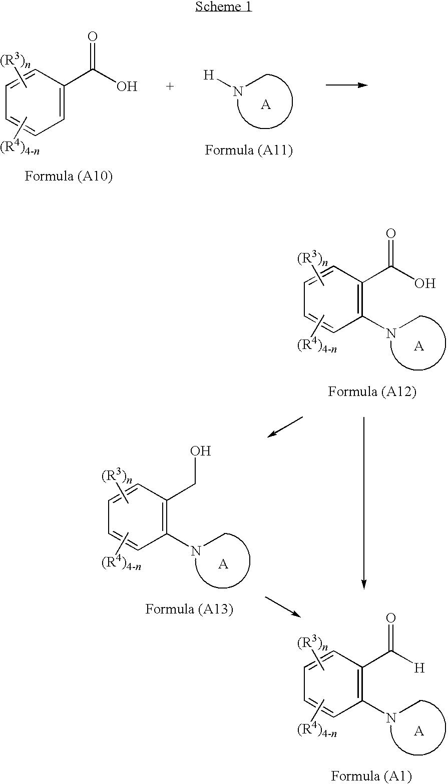

A compound of Formula (A10) may be reacted with a compound of Formula (A11) under standard conditions or in the presence of a suitable to provide a compound of Formula (A12). The carboxylic acid of the compound of Formula (A12) may be reduced directly to the aldehyde using, providing a compound of Formula (A1). Alternatively, the carboxylic acid of the compound of Formula (A12) may be first reduced to an alcohol, and subsequently oxidized to an aldehyde, providing a compound of Formula (A1).

Scheme 2 depicts a procedure by which compounds of Formula (A17), which are compounds of Formula (A3) in which R¹ and R² are H, may be prepared.

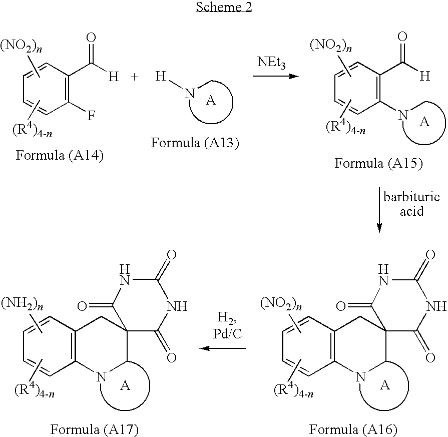

In any of the above-mentioned pharmaceutical compositions, processes, methods, uses, medicaments, and manufacturing features of the instant invention, any of the alternate embodiments of the compounds of the invention described herein also apply.

### EXAMPLES

The invention is now illustrated by, but not limited to, the following Examples, for which, unless otherwise stated:
(i) temperatures are given in degrees Celsius (° C.); operations are carried out at room temperature or ambient temperature, that is, in a range of 18-25° C.;
(ii) organic solutions were dried over anhydrous magnesium sulfate; evaporation of organic solvent was carried out using a rotary evaporator under reduced pressure (4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC or liquid chromatography/mass spectroscopy (LC/MS) and reaction times are given for illustration only;
(v) final products have satisfactory proton nuclear magnetic resonance (NMR) spectra and/or mass spectra data;
(vi) yields are given for illustration only and are not necessarily those which can be obtained by diligent process development; preparations were repeated if more material was required;
(vii) when given, NMR data is in the form of delta values for major diagnostic protons, given in part per million (ppm) relative to tetramethylsilane (TMS) as an internal standard, determined at 300 MHz in DMSO-d₆ unless otherwise stated;
(viii) chemical symbols have their usual meanings;
(ix) solvent ratio was given in volume:volume (v/v) terms.
(x) the following abbreviations may have been used:

| | |
|---|---|
| DMF | N,N-dimethylformamide; |
| THF | tetrahydrofuran; |
| DCM | dichloromethane; |
| DMAP | 4-dimethylaminopyridine; |
| DMSO | dimethylsulphoxide; |
| DIPEA | N,N-diisopropylethylamine; |
| EtOAc | ethyl acetate; and |
| IPA | isopropyl alcohol |

(xi) an ISCO Combiflash refers to flash chromatography on silica gel using Isco Combiflash® separation system: RediSep normal phase flash column, flow rate, 30-40 ml/min.

Unless otherwise indicated, it is believed that the Examples, and those Intermediates with chiral centers, were obtained as racemic mixtures.

Intermediate 1

### 2,4-Difluoro-5-iodo-benzoic acid

To an ice cooled and stirred solution of 2,4 difluorobenzoic acid (5.0 g, 31.6 mmol) in concentrated sulfuric acid (25 mL), was added N-iodosuccinamide (7.12 g, 31.6 mmol) in portions, and the mixture stirred 0-5° C. for 5 h during which time TLC shows the disappearance of starting material. The reaction mixture was poured into crushed ice with vigorous stirring. The precipitate thus formed was filtered, washed with cold water (5×50 mL), and dried under reduced pressure to give the title compound as a white solid. Yield: 8.5 g (94%).

¹H NMR (300 MHz, CDCl₃) δ: 6.65 (dd, 1H), 8.5 (m, 4H).

Intermediate 2

### 2,3-Difluoro-5-iodo-benzoic acid

To an ice cooled and stirred solution of 2,3 difluorobenzoic acid (10.0 g, 63.2 mmol) in concentrated sulfuric acid (50 mL), was added N-iodosuccinamide (14.2 g, 63.2 mmol) in portions, and the mixture stirred 0-5° C. for 5 hours during which time TLC showed the disappearance of the starting material. The reaction mixture was poured into crushed ice with vigorous stirring. The precipitate thus formed was filtered, washed with cold water (5×50 mL), and dried under reduced pressure to give the title compound as a white solid. Yield: 9.0 g (50%).

MS(ES)MH⁺: 283 for C₇H₃F₂IO₂

¹H NMR (400 MHz, CDCl₃) δ: 7.91 (m, 1H), 8.12(m, 1H), 13.83 (bs, 1H).

Intermediate 3

### (2,4-Difluoro-5-iodo-phenyl)-methanol

To an ice cooled and stirred solution of 2,4-difluoro-5-iodo-benzoic acid (Intermediate 1, 8.0 g, 28.1 mmol) in dry THF (120 mL), was added borane dimethyl sulfide (2.57 g, 33.8 mmol). The reaction mixture was heated at 80° C. for 2 hours, cooled to room temperature, and concentrated. The residue was dissolved in EtOAc (25 mL), washed with water (2×20 mL) followed by brine (2×20 mL), and dried over anhydrous sodium sulfate. The solution was filtered and the filtrate evaporated under reduced pressure. The residue thus obtained was purified over silica gel column using a gradient of EtOAc in petroleum ether to give the title product as a yellow solid. Yield: 7.4 g (97%).

¹H NMR (300 MHz, CDCl₃) δ: 4.45 (s, 2H), 7.2 (m, 1H), 7.8 (t, 1H).

Intermediate 4

### (2,3-Difluoro-5-iodo-phenyl)-methanol

To an ice cooled and stirred solution of 2,3-difluoro-5-iodo-benzoic acid (8.0 g, 28.1 mmol) in dry THF (120 mL), was added borane dimethyl sulfide (Intermediate 2, 2.57 g, 33.8 mmol) and the mixture heated at 80° C. for 2 hours. It was then cooled to room temperature and concentrated. The residue was dissolved in EtOAc (25 mL), washed with water (2×20 mL) followed by brine (2×20 mL) and dried over anhydrous sodium sulfate. It was filtered, and the filtrate was evaporated under reduced pressure. The residue thus obtained was purified over silica gel column using a gradient of ethylacetate in petroleum ether to give the title compound as a yellow solid. Yield: 6.0 g (62%).

MS (ES) MH⁺: 271 for C₇H₅F₂IO

¹H NMR (300 MHz, CDCl₃) δ: 4.5 (s, 2H), 7.6 (d, 1H), 7.7 (s, 1H), 10.2 (s, 1H).

Intermediate 5

### 2-((2R,6S)-2,6-Dimethyl-morpholin-4-yl)-3,4-difluoro-benzoic acid

To a stirred solution of 2,3,4-trifluorobenzoic acid (25.0 g, 142 mmol) in THF (250 mL), was added lithium bis(trimethylsilyl) amide (1M in THF) (156 mL, 156 mmol) at −78° C. under a nitrogen atmosphere, and the solution was stirred for 45 minutes. To this was added a premixed solution of cis-2,6-dimethylmorpholine (17.4 mL, 142 mmol) and lithium bis(trimethylsilyl)amide (1M in THF) (156 mL, 156 mmol) (which was stirred for 45 minutes at −78° C., before the addition) and stirring continued for 1 hour at −78° C. The reaction mixture was brought to room temperature and stirring continued for an additional 12 hours. Solvents were evaporated, and the residue was dissolved in ethyl acetate. It was washed with 1N HCl, followed by water and finally with brine. The combied organic layers were dried over anhydrous sodium sulfate and concentrated to give the title product as a semisolid. Yield: 27.5 g, (72%).

MS (ES) MH⁺: 271 for C₁₃H₁₅F₂NO₃

¹H NMR (DMSO-d₆): δ 1.2 (s, 6H), 2.9 (d, 2H), 3.1 (d, 2H), 3.9 (m, 2H), 7.2 (s, 1H), 7.3 (t, 1H), 8.1 (m, 1H).

Intermediate 6

### [2-((2R,6S)-2,6-Dimethyl-morpholin-4-yl)-3,4-difluoro-phenyl]-methanol

To a stirred and cold solution of 2-((2R,6S)-2,6-Dimethyl-morpholin-4-yl)-3,4-difluoro-benzoic acid (Intermediate 5, 27.0 g, 99.6 mmol) in THF (250 mL) was added sodium borohydride (12.56 g, 358.6 mmol) in small portions, followed by iodine (32.5 g, 139.4 mmol) in THF (250 mL). The addition was carried out such that the temperature was maintained below 10° C. The reaction mixture was brought to room temperature and refluxed for 12 hours. The reaction mixture was cooled and then quenched with methanol (250 mL). Solvents were evaporated and the residue treated with 2M sodium hydroxide solution (500 mL) for 2 hours. The aqueous layer was extracted with ethyl acetate (3×150 mL). The combined organic phases were washed with water followed by brine, dried over anhydrous sodium sulfate, and filtered. The filtrate was concentrated under reduced pressure to give the title product as a gummy solid. Yield: 27 g, (84%).

MS (ES) MH⁺: 257 for C₁₃H₁₇F₂NO₂

¹H NMR (DMSO-d₆): ¹H NMR (400 MHz, CDCl₃) δ: 1.2 (s, 6H), 3.0 (d, 3H), 3.1 (d, 2H), 3.9 (m, 2H), 4.78 (s, 2H), 6.9 (d, 1H), 7.0 (t, 1H).

Intermediate 7

### (2R,6S)-4-[6-(tert-Butyl-diphenyl-silanyloxymethyl)-2,3-difluoro-phenyl]-2,6-dimethyl-morpholine

To an ice cooled and stirred solution of [2-((2R,6S)-2,6-Dimethyl-morpholin-4-yl)-3,4-difluoro-phenyl]-methanol (Intermediate 6, 27.0 g, 105 mmol) in CH₂Cl₂ was added imidazole (8.5 g, 126 mmol), followed by t-butyl-chloro-diphenylsilane (30 mL, 115 mmol) over a period of 15 minutes. The reaction mixture was brought to room temperature and stirred for 12 hours, during which time TLC showed the disappearance of the starting material. The reaction mixture was diluted with CH₂Cl₂ and washed successively with 1 N HCl (1×250 mL), water and brine. The organic layer was dried over anhydrous sodium sulfate, filtered and concentrated. The residue thus obtained was purified over silica gel flash column using a gradient of ethyl acetate in petroleum ether to give the title product as a white solid. Yield: 45 g (94%).

MS (ES) MH⁺: 496 for C₂₉H₃₅F₂NO₂Si

¹HNMR (400 MHz, CDCl₃) δ: 1.1 (s, 15H); 2.6 (d, 2H); 2.8 (m, 2H); 3.5 (t, 2H); 4.7 (s, 2H); 7.0 (q, 1H); 7.3 (t, 1H); 7.4 (m, 10H).

Intermediate 8

### 5-(tert-Butyl-diphenyl-silanyloxymethyl)-4-((2R,6S)-2,6-dimethyl-morpholin-4-yl)-2,3-difluoro-benzaldehyde

To a stirred solution of (2R,6S)-4-[6-(tert-Butyl-diphenyl-silanyloxymethyl)-2,3-difluoro-phenyl]-2,6-dimethyl-morpholine (Intermediate 7, 15.0 g, 30.0 mmol) in THF (150 mL) was added s-butyllithium (1.4 M in cyclohexane, 66.4 mL, 93 mmol) at −78° C. under a nitrogen atmosphere. After stirring for 1 hour at this temperature, DMF (3.4 mL, 45 mmol) was added dropwise. The addition of DMF was carried out such that the temperature was maintained below −60° C. After stirring for 30 minutes, TLC showed the disappearance of starting material. The reaction mixture treated with saturated aqueous NH₄Cl solution and the aqueous layer extracted by EtOAc (2×100 mL). The combined organic layers were dried over anhydrous sodium sulfate, filtered, and the filtrate concentrated. The residue thus obtained was purified over silica gel flash column using a gradient of ethyl acetate in petroleum ether to give the title product as a yellow solid. Yield: 13.2 g (84%). MS (ES) MH⁺: 524 for C₃₀H₃₅F₂NO₃Si

¹H NMR (400 MHz, CDCl₃) δ: 1.1 (s, 15H), 2.8 (m, 4H), 3.4 (m, 2H), 4.6 (s, 2H), 7.3 (t, 4H), 7.4 (t, 2H), 7.6 (d, 4H), 7.8 (s, 1H), 10.2 (s, 1H).

Intermediate 9

### 4-((2R,6S)-2,6-Dimethyl-morpholin-4-yl)-2,3-difluoro-5-hydroxymethyl-benzaldehyde

A mixture of 5-(tert-Butyl-diphenyl-silanyloxymethyl)-4-((2R,6S)-2,6-dimethyl-morpholin-4-yl)-2,3-difluoro-benzaldehyde (Intermediate 8, 7.2 g, 28 mmol) and 4N HCl in dry dioxane (75 mL) was stirred at room temperature for 12 hours during which time TLC showed the deprotection of the TBDPS group. The reaction mixture was treated with cold water (20 mL) and extracted with ethyl acetate (3×50 mL). The organic phases were combined, dried over anhydrous sodium sulfate, and filtered. The filtrate was concentrated under reduced pressure. The residue thus obtained was purified over a silica gel flash column using a gradient of ethyl acetate in petroleum ether to give the title product as a yellow solid. Yield: 3.4 g (42%).

MS (ESP): 285 for C₁₄H₁₇F₂NO₃

¹H NMR (400 MHz, CDCl₃) δ: 1.2 (s, 6H), 3.0 (d, 4H), 3.8 (m, 2H), 4.7 (s, 1H), 7.6 (d, 1H), 10.2 (s 1H).

Intermediate 10

### 1-[5-(tert-Butyl-diphenyl-silanyloxymethyl)-4-((2R,6S)-2,6-dimethyl-morpholin-4-yl)-2,3-difluoro-phenyl]-ethanone

To a stirred solution of (2R,6S)-4-[6-(tert-Butyl-diphenyl-silanyloxymethyl)-2,3-difluoro-phenyl]-2,6-dimethyl-morpholine (Intermediate 7, 20.0 g, 30.0 mmol) in THF (150 mL) was added sec-butyllithium (1.4 M in cyclohexane, 66.4 mL, 93 mmol) at −78° C. under a nitrogen atmosphere. After stirring for 1 hour at this temperature, N-methoxy-N-methyl-acetamide (3.4 mL, 45 mmol) was added. After stirring for 30 minutes at −78° C., the solution was allowed reach room temperature and stirring was continued for 12 hours. The reaction mixture treated with saturated aqueous NH₄Cl solution and the aqueous layer extracted by EtOAc (2×100 mL). Organic phases were combined and dried over anhydrous sodium sulfate. The solution was filtered and the filtrate concentrated. The residue thus obtained was purified over a silica gel flash column using a gradient of ethyl acetate in petroleum ether to give the title compound as a yellow solid. Yield: 13.2 g (84%). MS(ES)MH⁺: 538.6 for C₃₁H₃₇F₂NO₃Si

¹H NMR (400 MHz, CDCl₃) δ: 1.1 (s, 15H), 2.8 (m, 4H), 3.4 (m, 2H), 4.6 (s, 2H), 7.3 (t, 4H), 7.4 (t, 2H), 7.6 (d, 4H), 7.8 (s, 1H), 10.2 (s, 1H).

Intermediate 11

### [5-(tert-Butyl-diphenyl-silanyloxymethyl)-4-((2R,6S)-2,6-dimethyl-morpholin-4-yl)-2,3-difluoro-phenyl]-methanol

To an ice cooled solution of 5-(tert-Butyl-diphenyl-silanyloxymethyl)-4-((2R,6S)-2,6-dimethyl-morpholin-4-yl)-2,3-difluoro-benzaldehyde (Intermediate 8, 7.9 g, 15.1 mmol) in methanol (80 mL) was added sodium borohydride (2.06 g, 54.4 mmol). The reaction mixture was stirred for 2 hours during which time TLC showed the disappearance of starting material. The reaction mixture was quenched with acetone and diluted with ethyl acetate. The organic phase was washed with water (2×25 mL) followed by brine (2×25 mL), and then dried over anhydrous sodium sulfate. The organic layer was then was filtered, and the filtrate was concentrated under reduced pressure to give the title compound as a gummy solid. Yield: 7.9 g (98%).

MS (ES) MH⁺: 526 for C₃₀H₃₇F₂NO₃Si

¹H NMR (400 MHz, CDCl₃) δ: 1.1 (s, 15H), 2.5 (d, 2H), 2.7 (t, 2H), 2.8 (s, 2H), 4.7 (s, 2H), 5.2 (t 1H), 7.3 (t, 1H), 7.4 (s, 10H).

Intermediate 12

### 5-({[tert-Butyl(diphenyl)silyl]oxy}methyl)-4-[(2R,6S)-2,6-dimethyl morpholin-4-yl]-2,3-difluorobenzyl acetate

To an ice cold solution of [5-(tert-Butyl-diphenyl-silanyloxymethyl)-4-((2R,6S)-2,6-dimethyl-morpholin-4-yl)-2,3-difluoro-phenyl]-methanol (Intermediate 11, 7.9 g, 15.04 mmol) in anhydrous DCM was added pyridine (6.06 mL, 75.2 mmol) followed by acetic anhydride (3.1 mL, 33.10 mmol). It was allowed to reach room temperature and stirred for 12 hours. Solvents were evaporated and the residue treated with 5% citric acid (250 mL). The aqueous layer extracted with ethyl acetate (3×150 mL) and combined. The combined organic phase washed with water followed by brine and then dried over anhydrous sodium sulfate. The combined organic layers were filtered and concentrated under reduced pressure to give the title compound as a gummy solid. Yield 7.8 g (94%).

MS (ES) MH⁺: 568 (M+H) for C₃₂H₃₉F₂NO₄Si

¹H NMR (400 MHz, CDCl₃) δ: 1.0 (s, 15H), 2.1 (s, 3H), 2.5 (d, 2H), 2.8 (t, 2H), 3.4 (s, 2H), 4.7 (s, 2H), 5.3 (s, 2H), 7.1 (t, 1H), 7.6 (s, 10H).

Intermediate 13

### 4-[(2R,6S)-2,6-Dimethylmorpholin-4-yl]-2,3-difluoro-5-(hydroxymethyl)benzyl acetate

A mixture of 5-({[tert-butyl(diphenyl)silyl]oxy}methyl)-4-[(2R,6S)-2,6-dimethyl morpholin-4-yl]-2,3-difluorobenzyl acetate (Intermediate 12, 7.8 g, 13.7 mmol) and HCl in dry dioxane (75 mL) was stirred at room temperature for 12 hours during which time TLC showed the deprotection of TBDPS group. The reaction mixture was treated with cold water (20 mL) and extracted with ethyl acetate (3×50 mL). The combined organic layers were dried over anhydrous sodium sulfate, filtered, concentrated under reduced pressure. The residue thus obtained was purified over silica gel flash column using a gradient of ethyl acetate in petroleum ether to give the title compound as an off white solid. Yield: 3.2 g (71%).

MS(ES)MH⁺: 330 for C₁₆H₂₁F₂NO₄

¹H NMR (400 MHz, CDCl₃) δ: 1.2 (d, 6H), 2.1 (s, 3H), 2.8 (d, 2H), 2.9 (t, 2H), 3.7 (s,2H), 4.7 (s, 2H), 5.1 (s, 2H), 7.2 (t, 1H).

Intermediate 14

### 1-[4-((2R,6S)-2,6-Dimethyl-morpholin-4-yl)-2,3-difluoro-5-hydroxymethyl-phenyl]-ethanone

The title compound was synthesized from 1-[5-(tert-Butyl-diphenyl-silanyloxymethyl)-4-((2R,6S)-2,6-dimethyl-morpholin-4-yl)-2,3-difluoro-phenyl]-ethanone (Intermediate 10), using a method similar to the one described for the synthesis of Intermediate 13.

MS(ES)MH⁺: 300 for C₁₅H₁₉F₂NO₃

¹H NMR (400 MHz, CDCl₃) δ: 1.1 (s, 15H), 2.5 (d, 2H), 2.7 (t, 2H), 2.8 (s, 2H), 4.7 (s,2H), 5.2 (t 1H), 7.3 (t, 1H), 7.4 (s, 10H).

Intermediate 15

### 4-[(2R,6S)-2,6-Dimethylmorpholin-4-yl]-2,3-difluoro-5-formylbenzyl acetate

To an ice-cooled solution of 4-[(2R,6S)-2,6-dimethylmorpholin-4-yl]-2,3-difluoro-5-(hydroxymethyl)benzyl acetate (Intermediate 13, 3.2 g, 9.72 mmol) in DCM/CH₃CN mixture (20 mL, 1:1 v/v) was added NMO (2.2 g, 19.44 mmol) followed TPAP (340 mg, 0.97 mmol) and the reaction mixture was stirred for 2 hours at room temperature. The reaction mixture was filtered thorough a silica gel bed and washed with EtOAc. The organic phase was concentrated under reduced pressure to give the title product as a yellow solid. Yield: 3.0 g (94%).

MS(ES)MH⁺: 328 for C₁₆H₁₉F₂NO₄

¹H NMR (400 MHz, CDCl₃): δ 1.2 (d, 6H); 2.1 (s, 3H), 3.0 (d, 2H), 3.1 (t, 2H), 3.8 (s, 2H), 5.1 (s, 2H), 7.6 (t 1H), 10.2 (s, 1H).

Intermediates 16 to 22 were synthesized from the indicated starting materials, using a method similar to the one described for the synthesis of Intermediate 15.

Intermediate 16

### 2,4-Difluoro-5-iodo-benzaldehyde

Starting material: (2,4-Difluoro-5-iodo-phenyl)-methanol (Intermediate 3).

¹H NMR (300 MHz, CDCl₃) δ: 6.9 (m, 1H), 8.3 (m, 1H), 10.2 (s, 1H).

Intermediate 17

### 2,3-Difluoro-5-iodo-benzaldehyde

Starting material: (2,3-Difluoro-5-iodo-phenyl)-methanol (Intermediate 4).

¹H NMR (300 MHz, CDCl₃) δ: 7.76(s, 1H), 7.95(s, 1H), 10.25 (s, 1H).

Intermediate 18

### 5-Acetyl-2-((2R,6S)-2,6-dimethyl-morpholin-4-yl)-3,4-difluoro-benzaldehyde

Starting material: [4-((2R,6S)-2,6-Dimethyl-morpholin-4-yl)-2,3-difluoro-5-hydroxymethyl-phenyl]-ethanone (Intermediate 14).

MS(ES) MH⁺: 298.2 for C₁₅H₁₇F₂NO₃

¹H-NMR (400 MHz, CDCl₃): δ 1.2 (d, 6H); 2.1 (s, 3H), 3.0 (d, 2H), 3.1 (t, 2H), 3.8 (s, 2H), 5.1 (s, 2H), 7.6 (t 1H), 10.2 (s, 1H).

Intermediate 19

### 4-((2R,6S)-2,6-Dimethyl-morpholin-4-yl)-2,3-difluoro-5-formyl-benzoic acid

Starting Material: 4-((2R,6S)-2,6-Dimethyl-morpholin-4-yl)-2,3-difluoro-5-hydroxy methyl-benzoic acid Intermediate 27.

MS (ES)MH⁺: 300.2 for C₁₄H₁₅F₂NO₄

¹H NMR (400 MHz, CDCl₃) δ: 1.1 (d, 6H); 2.6 (d, 2H); 2.8 (m, 2H); 3.5 (t, 2H); 7.2(s, 1H); 10.0 (s, 1H).

Intermediate 20

### 2-((2R,6S)-2,6-Dimethyl-morpholin-4-yl)-3,4-difluoro-5-[(Z)-pyrrolidin-1-ylimino methyl]-benzaldehyde

Starting material: {2-((2R,6S)-2,6-Dimethyl-morpholin-4-yl)-3,4-difluoro-5-[(Z)-pyrrolidin-1-yliminomethyl]-phenyl}-methanol (Intermediate 35).

MS(ES)MH⁺: 354.4 for C₁₈H₂₅F₂N₃O₂

¹H NMR (400 MHz, CDCl₃)δ: 1.21 (d, 6H), 2.38 (s, 3H), 3.06 (d, 4H), 3.94 (m, 2H), 5.39 (s, 2H), 7.57 (d,1H), 7.89 (s, 1H).

Intermediate 21

### 2-[(2R,6S)-2,6-Dimethylmorpholin-4-yl]-3,4-difluoro-5-[(E)-(morpholin-4-ylimino)methyl]benzaldehyde

Starting Material: {2-[(2R,6S)-2,6-Dimethylmorpholin-4-yl]-3,4-difluoro-5-[(E)-(morpholin-4-ylimino)methyl]phenyl}methanol (Intermediate 36).

MS(ES)MH⁺: 368.4 for C₁₈H₂₃F₂N₃O₃

¹H NMR (400 MHz, CDCl₃) δ: 1.21 (d, 6H), 3.04 (m, 4H), 3.22 (t, 4H), 3.83-3.91 (m, 6H), 7.62 (s, 1H), 8.14 (s, 1H), 10.30 (s, 1H).

Intermediate 22

### N′-[(E)-{4-[(2R,6S)-2,6-Dimethylmorpholin-4-yl]-2,3-difluoro-5-formyl phenyl}methylidene]acetohydrazide

Starting Material: N′-[(E)-{4-[(2R,6S)-2,6-Dimethylmorpholin-4-yl]-2,3-difluoro-5-(hydroxymethyl)phenyl}methylidene]acetohydrazide (Intermediate 37).

MS(ES)MH⁺: 340.4 for C₁₆H₁₉F₂N₃O₃.

Intermediate 23

### [(2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]methyl acetate

To a solution of 4-[(2R,6S)-2,6-dimethylmorpholin-4-yl]-2,3-difluoro-5-formylbenzyl acetate (Intermediate 15, 3.0 g, 9.17 mmol) in IPA was added barbituric acid (1.4 g, 11.00 mmol) and the mixture heated at 85° C. for 12 hours. Solvents were evaporated and the residue thus obtained was purified over neutral alumina using a gradient of methanol in DCM to give the title compound as an off white solid. Yield: 3.0 g (75%).

MS(ES)MH⁺: 438 for C₂₀H₂₁F₂N₃O₆

¹H NMR (400 MHz, DMSO-d₆): δ 1.9 (d, 3H); 2.0 (d, 3H), 2.4 (s, 3H), 2.8 (d, 2H), 3.0 (t, 1H), 3.1 (t, 1H), 3.4 (s, 1H), 3.6 (t, 2H), (3.9 (d, 2H), 4.0 (d, 1H), 6.8 (d, 1H), 11.4 (s, 1H), 11.7 (s, 1H).

Intermediate 24

### (2R,4S,4aS)-rel-9,10-Difluoro-8-(hydroxymethyl)-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Dry ammonia gas was purged through a stirred solution of [(2R,4S,4aS)-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]methyl acetate (Intermediate 23, 3.0 g, 6.86 mmol) in MeOH/THF (40 mL, 1:1 v/v) at −78° C., for 15 minutes. The reaction mixture was allowed to reach room temperature and then stirred for 24 hours. The reaction mixture was then cooled to −10° C. and purged with nitrogen to remove excess ammonia and concentrated. The residue was purified over silica gel column using a gradient of methanol in chloroform, to give the title compound as a pale yellow solid. Yield: 2.2 g (81%).

MS(ES) MH⁺: 396.4 for C₁₈H₁₉F₂N₃O₅

¹H NMR (400 MHz, DMSO-d₆): δ 0.9 (d, 3H); 1.2 (d, 3H), 2.8 (d, 1H), 2.9 (t, 1H), 3.3 (t, 1H), 3.6 (m, 1H), 3.9 (s, 2H), 4.3 (d, 1H), 4.9 (d, 2H), 5.1 (t, 1H), 6.7 (d, 1H), 11.4 (s, 1H), 11.7 (s, 1H).

Intermediate 25

### (2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidine]-8-carbaldehyde

To an ice-cooled solution of (2R,4S,4aS)-9,10-difluoro-8-(hydroxymethyl)-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione (Intermediate 24, 2.2 g, 5.59 mmol) in DCM/CH₃CN mixture (20 mL, 1:1 v/v) was added NMO (1.3 g, 11.19 mmol), followed by TPAP (0.19 g, 0.55 mmol). The resulting mixture was stirred for 2 hours at room temperature. The reaction mixture was filtered thorough a silica gel bed and washed with EtOAc. The organic phase was concentrated under reduced pressure to give the title compound as a yellow crystalline solid. Yield: 2.0 g (91%).

MS(ESP): 394.4 for C₁₈H₁₇F₂N₃O₅

¹H NMR (400 MHz, DMSO-d₆): δ 0.9 (d, 3H); 1.2 (d, 3H), 2.5 (t, 1H), 3.1 (t, 1H), 3.5 (t, 2H), 3.6 (s, 1H), 3.7 (d, 1H), 4.0 (t, 1H), 4.2 (d, 1H), 7.2 (d, 1H), 9.8 (s, 1H), 11.5 (s, 1H), 11.8 (s, 1H).

Intermediate 26

### 5-(tert-Butyl-diphenyl-silanyloxymethyl)-4-((2R,6S)-2,6-dimethyl-morpholin-4-yl)-2,3-difluoro-benzoic acid

To a stirred solution of (2R,6S)-4-[6-(tert-Butyl-diphenyl-silanyloxymethyl)-2,3-difluoro-phenyl]-2,6-dimethyl-morpholine (Intermediate 7, 15.0 g, 30.0 mmol) in THF (150 mL) was added sec-butyllithium (1.4 M in cyclohexane, 66.4 mL, 93 mmol) at −78° C. under a nitrogen atmosphere. After stirring for 2 hours at −78° C., the reaction mixture was quenched with dry ice (˜2.0 g), and slowly warmed to 0° C. and further stirred for 12 hours. The reaction mixture was concentrated under the reduced pressure and the residue dissolved in water (150 mL). The resulting solution was acidified to pH ˜4 using 1N HCl and extracted with EtOAc (3×100 mL). The combined organic layers were dried over anhydrous sodium sulfate and concentrated under reduced pressure. The residue thus obtained was purified over silica gel flash column using a gradient of ethyl acetate in petroleum ether to give the title compound as white solid. Yield: 13.2 g (84%)

MS (ES) MH⁺: 540.6 for C₃₀H₃₅F₂NO₄Si

¹H NMR (400 MHz, CDCl₃) δ: 1.1 (s, 15H), 2.6 (d, 2H), 2.8 (m, 2H), 3.5 (t, 2H), 4.7 (s, 2H), 7.2(s, 1H), 7.3 (t, 6H), 7.5 (d, 4H).

Intermediate 27

### 4-((2R,6S)-2,6-Dimethyl-morpholin-4-yl)-2,3-difluoro-5-hydroxymethyl-benzoicacid

A mixture of 5-(tert-Butyl-diphenyl-silanyloxymethyl)-4-((2R,6S)-2,6-dimethyl-morpholin-4-yl)-2,3-difluoro-benzoic acid (Intermediate 26, 7.2 g, 28 mmol) and HCl in anhydrous dioxane (75 mL) was stirred at room temperature for 12 hours during which time TLC showed the deprotection of the TBDPS group. The reaction mixture was concentrated under reduced pressure. The residue thus obtained was purified over silica gel flash column using a gradient of ethyl acetate in petroleum ether to give the title compound as a yellow solid. Yield: 3.4 g (42%)

MS (ES)MH⁺: 302.2 for C₁₄H₁₇F₂NO₄

¹H NMR (400 MHz, CDCl₃) δ: 1.1 (d, 6H); 2.6 (d, 2H); 2.8 (m, 2H); 3.5 (t, 2H); 4.7 (s, 2H); 7.2(s, 1H).

Intermediate 28

### (2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidine]-8-carboxylic acid

To a solution of 4-((2R,6S)-2,6-Dimethyl-morpholin-4-yl)-2,3-difluoro-5-formyl-benzoic acid (Intermediate 19, 100 mg, 0.272 mmol) in IPA was added barbituric acid (34 mg, 0.272 mmol), and the mixture was heated at 85° C. for 12 hours. The solvents were evaporated and the residue thus obtained was purified over neutral alumina using a gradient of methanol in DCM to give the title compound as a pale yellow solid. Yield: 32 mg (32%).

MS (ES)MH⁺: 410.2 for C₁₈H₁₇F₂N₃O₆

¹H NMR (400 MHz, CDCl₃) δ: 1.1 (d, 6H); 3.3 (d, 3H); 3.8 (m, 2H); 4.2 (s, 2H); 7.2(s, 1H).

Intermediate 29

### (2R,4S,4aS)-rel-8-Acetyl-9,10-difluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

The title compound was synthesized from 5-Acetyl-2-((2R,6S)-2,6-dimethyl-morpholin-4-yl)-3,4-difluoro-benzaldehyde (Intermediate 18), using a method similar to the one described for the synthesis of Intermediate 28.

MS(ES)MH⁺: 408.2 for C₁₉H₁₉F₂N₃O₅

¹H NMR (400 MHz, DMSO-d₆) δ: 0.8 (d, 3H), 1.1 (d, 3H), 2.1 (s, 3H), 2.8 (d, 1H), 3.0 (t, 1H), 3.5 (d, 1H), 3.6 (m, 1H), 3.7 (d, 1H), 3.9 (d, 1H), 4.1 (d, 1H), 7.2 (d, 1H), 11.5 (s, 1H), 11.8 (s,1H).

Intermediate 30

### 2-[(2R,6S)-2,6-Dimethylmorpholin-4-yl]-5-nitrobenzaldehyde

To a stirred solution of 2-fluoro-5-nitro-benzaldehyde (5 g, 29.6 mmol) in dry acetonitrile (50 mL), was added triethylamine (8.98 g, 88.6 mmol) followed by cis-2,6 dimethyl morpholine (4.09 g, 35.5 mmol). The reaction mixture was heated at 85° C. for 12 hours under a nitrogen atmosphere, cooled to room temperature, quenched with sat. NaHCO₃ solution (25 mL), and extracted with ethyl acetate (3×25 mL). The combined organic layers were washed with water, dried over anhydrous Na₂SO₄, and evaporated under reduced pressure to give the title compound as a yellow solid. (Yield: 7.3 g, 93.5%). MS(ES) MH⁺: 265.2 for C₁₃H₁₆N₂O₄

¹H NMR (400 MHz, CDCl₃) δ: 1.27 (d, 6H), 2.84 (t, 2H), 3.34 (d, 2H), 3.91-3.96 (m, 2H), 7.09 (d, 1H), 8.34 (dd, 1H), 8.65 (s, 1H), 10.08 (s, 1H).

Intermediates 31 and 32 were synthesized from the indicated starting materials, using a method similar to the one described for the synthesis of Intermediate 30.

Intermediate 31

### 2-((2S,6R)-2,6-Dimethyl-morpholin-4-yl)-4-fluoro-5-iodo-benzaldehyde

Starting material: 2,4-Difluoro-5-iodo-benzaldehyde (Intermediate 16).

MS (ES)MH⁺: 364.0 for C₁₃H₁₅F₁INO₂

¹H NMR (300 MHz, CDCl₃) δ: 1.22 (d, 6H), 2.63 (t, 2H), 3.09 (d, 2H), 3.98 (m, 2H), 6.75 (d, 1H), 8.15 (d, 1H), 10.08 (s, 1H).

Intermediate 32

### 2-((2S,6R)-2,6-Dimethyl-morpholin-4-yl)-3-fluoro-5-iodo-benzaldehyde

Starting material: 2,3-Difluoro-5-iodo-benzaldehyde (Intermediate 17).

¹H NMR (300 MHz, CDCl₃) δ: 7.76(s, 1H), 7.95(s, 1H), 10.25 (s, 1H).

Intermediate 33

### (2R,4S,4aS)-2,4-Dimethyl-8-nitro-1,2,4,4a-tetrahydro-2′H,6H-spiro[1,4-oxazino[4,3-a]quinoline-5,5′-pyrimidine]-2′,4′,6′(1′H,3′H)-trione

To a stirred solution of 2-[(2R,6S)-2,6-dimethylmorpholin-4-yl]-5-nitrobenzaldehyde (Intermediate 30, 7.3 g, 27.6 mmol) in dry IPA (150 mL) was added barbituric acid (3.89 g, 30.4 mmol) and heated to 80° C., overnight under a nitrogen atmosphere. The reaction mixture was cooled to room temperature and filtered. The solid thus obtained was stirred with water (30 mL) for 2 hours, filtered, and dried to give the title compound as a yellow solid. (Yield: 9 g, 90%).

MS(ES) MH⁺: 375.3 for C₁₇H₁₈N₄O₆

¹H NMR (400 MHz, DMSO-d₆) δ: 0.9 (d, 6H), 1.15 (d, 6H), 2.9 (d, 1H), 2.9-3.0 (m, 1H), 3.5-3.6 (m, 3H), 3.9 (d, 1H), 4.26-4.3 (m, 1H), 7.0 (d, 1H), 7.8 (d, 1H), 7.95 (dd, 1H).

Intermediate 34

### (2R,4S,4aS)-rel-8-Amino-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

To a solution of (2R,4S,4aS)-2,4-dimethyl-8-nitro-1,2,4,4a-tetrahydro-2′H,6H-spiro[1,4-oxazino[4,3-a]quinoline-5,5′-pyrimidine]-2′,4′,6′(1′H,3′H)-trione (Intermediate 33, 500 mg, 1.33 mmol) in anhydrous THF, was added Rancy Ni (20%, 100 mg, rinsed with THF) and the mixture hydrogenated under a positive pressure of hydrogen (1 Kg) for 16 hours. The reaction mixture was filtered through Celite, and the filtrate taken for the next step without further isolation of the title product.

Intermediate 35

### {2-((2R,6S)-2,6-Dimethyl-morpholin-4-yl)-3,4-difluoro-5-[(Z)-pyrrolidin-1-yliminomethyl]-phenyl}-methanol

To a solution of intermediate 4-((2R,6S)-2,6-Dimethyl-morpholin-4-yl)-2,3-difluoro-5-hydroxymethyl-benzaldehyde (Intermediate 9, 250 mg, 0.87 mmol) in EtOH, was added N-amino morpholine (89 mg, 0.87 mmol) followed by glacial acetic acid (2 drops). The reaction mixture washeated to 85° C. for 12 hours. The mixture was then concentrated under reduced pressure to give the title compound as a pale yellow solid which was used for the next step without further purification. Yield: 120 mg (39%).

MS(ES)MH⁺: 354.4 for C₁₈H₂₅F₂N₃O₂

¹H NMR (400 MHz, CDCl₃)δ: 1.21 (d, 6H), 2.38 (s, 3H), 3.06 (d, 4H), 3.94 (m, 2H), 5.39 (s, 2H), 7.57 (d,1H), 7.89 (s, 1H),

Intermediates 36 and 37 were synthesized from 4-((2R,6S)-2,6-Dimethyl-morpholin-4-yl)-2,3-difluoro-5-hydroxymethyl-benzaldehyde (Intermediate 9) and the indicated starting materials using a method similar to the one described for the synthesis of Intermediate 35.

Intermediate 36

### {2-[(2R,6S)-2,6-Dimethylmorpholin-4-yl]-3,4-difluoro-5-[(E)-(morpholin-4-ylimino)methyl]phenyl}methanol

Starting material: 4-Aminomorpholine

MS(ES)MH⁺: 370.4 for C₁₈H₂₅F₂N₃O₃

¹H NMR (400 MHz, CDCl₃)δ: 1.21 (d, 6H), 2.38 (s, 3H), 3.06 (d, 4H), 3.94 (m, 2H), 5.39 (s, 2H), 7.57 (d,1H), 7.89 (s, 1H), 8.92 (s, 1H).

Intermediate 37

### N′-[(E)-{4-[(2R,6S)-2,6-Dimethylmorpholin-4-yl]-2,3-difluoro-5-(hydroxymethyl)phenyl}methylidene]acetohydrazide

Starting material: Acetylhydrazide

MS(ES)MH⁺: 342.4 for C₁₆H₂₁F₂N₃O₃

¹H NMR (400 MHz, CDCl₃)δ: 1.21 (d, 6H), 2.38 (s, 3H), 3.06 (d, 4H), 3.94 (m, 2H), 5.39 (s, 2H), 7.57 (d,1H), 7.89 (s, 1H), 8.94(s, 1H)

Intermediate 38

### 5-Bromo-2-((2R,6S)-2,6-dimethylmorpholino)benzaldehyde

A solution of 5-bromo-2-fluorobenzaldehyde (4.62 g, 22.76 mmol), cis-2,6-dimethylmorpholine (3.08 mL, 25.03 mmol), and N-ethyldiisopropylamine (5.91 mL, 34.14 mmol) in acetonitrile (50 mL) was heated at reflux for 3 hours. Additional cis-2,6-dimethylmorpholine (3.08 mL, 25.03 mmol) was added and heating at reflux was continued overnight. Solvent was removed and the mixture was partitioned between EtOAc and 1N HCl. The EtOAc was separated and washed with brine. The combined aqueous layers were twice more extracted with EtOAc with each extract being washed with water and brine. Drying (MgSO₄) and removal of solvent gave an oil. The crude product was chromatographed on silica gel (100% CH₂Cl₂ followed by gradient elution to 10% MeOH in CH₂Cl₂) to give the title product as a yellow solid.

MS (ES) MH⁺: 298 for C₁₃H₁₆BrNO₂

¹H NMR (DMSO-d₆): 1.2 (d, 6H), 2.6 (t, 2H), 3.0 (d, 2H), 3.9 (m, 2H), 7.0 (d, 1H), 7.6 (d, 1H), 7.9 (s, 1H), 10.2 (s, 1H).

Intermediate 39

### 2-[(2R,6S)-2,6-Dimethylmorpholin-4-yl]-5-iodobenzaldehyde

2-Fluoro-5-iodobenzaldehyde and (2R,6S)-2,6-dimethylmorpholine were reacted in a procedure similar to the one described for the synthesis of Intermediate 38, providing the title compound.

MS (ES) M+H⁻: 346 for C₁₃H₁₆INO₂

¹H NMR (400 MHz, CDCl₃) δ: 1.23 (d, J=4.0 Hz, 6H), 2.66 (t, 2H), 3.05 (d, J=8.0 Hz, 2H), 3.88-3.93 (m, 2H), 6.86 (d, J=8.0 Hz, 1H), 7.80 (dd, J=8.0 Hz, J=8.0 Hz, 1H), 8.08 (s, 1H), 10.17 (s, 1H).

Intermediate 40

### (2R,4S,4aS)-rel-8-Bromo-2,4-dimethyl-2,4,4a,6-tetrahydro-1H,1′H-spiro[[1,4]oxazino[4,3-a]quinoline-5,5′-pyrimidine]-2′,4′,6′(3′H)-trione

A solution of 5-bromo-2-((2R,6S)-2,6-dimethylmorpholino)benzaldehyde (Intermediate 38, 2.106 g, 7.06 mmol) and barbituric acid (0.905 g, 7.06 mmol) in toluene (30 mL) was stirred at room temperature overnight. The mixture was heated at 80° C. for 2 hours and then at reflux for 2 additional hours. The solution was cooled to room temperature, after which the solids were filtered, rinsed with toluene, and then with ether before being dried in vacuo to give 2.5 g of the title product.

MS (ES) MH⁺: 408 for C₁₇H₁₈BrN₃O₄

¹H NMR (DMSO-d₆): 0.9 (d, 3H), 1.1 (d, 3H), 2.7-2.9 (m, 2H), 3.2 (m, 1H), 3.4-3.7 (m, 3H), 4.0 (d, 1H), 6.8 (d, 1H), 7.0 (s, 1H), 7.2 (d, 1H), 11.5 (s, 1H), 11.8 (s, 1H).

Intermediate 41

### (2R,4S,4aS)-rel-8-Iodo-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

The title compound was synthesized from 2-[(2R,6S)-2,6-Dimethylmorpholin-4-yl]-5-iodobenzaldehyde (Intermediate 39) and barbituric acid in isopropanol, using a method similar to the one described for the synthesis of Intermediate 40.

MS(ES) MH⁺: 455.9 for C₁₇H₁₈IN₃O₄

¹H NMR (400 MHz, DMSO-d₆) d: 0.89 (d, J=4.0 Hz, 3H), 1.11 (d, J=4.0 Hz, 3H), 2.84-2.75 (m, 2H), 3.28 (d, J=16.0 Hz, 1H), 3.50-3.47 (m, 1H), 3.58-3.52 (m, 1H), 3.66 (d, J=12.0 Hz, 1H), 3.96 (dd, J=12.0 Hz, J=16.0 Hz, 1H), 6.69 (d, J=8.0 Hz, 1H), 7.15 (s, 1H),7.32 (d, J=4.0 Hz,1H), 11.44 (s, 1H), 11.74 (s, 1H).

Intermediate 42

### 5-Bromo-2,3,4-trifluoro-benzaldehyde

To a solution of diisopropylamine (1.05 g, 10.46 mmol) in THF (10 mL) was added n-butyllithium (6.5 mL, 1.6 N), dropwise at −10° C. and the solution stirred for 30 min at −10° C. The mixture was cooled to −78° C. and to this was added 1-bromo-2,3,4-trifluorobenzene (1.0 g, 4.76 mmol) in THF (10 mL) with stirring at −78° C. under a nitrogen atmosphere. After stirring for 1 hour at this temperature, DMF (3.4 mL, 45 mmol) was added dropwise so that the temperature was maintained below −60° C. The reaction mixture was slowly allowed to warm to room temperature, and stirred overnight. The reaction mixture treated with saturated aqueous NH₄Cl solution and the aqueous layer extracted by EtOAc (2×100 mL). The combined organic phases were dried over anhydrous sodium sulfate, filtered and concentrated. The residue thus obtained was purified over silica gel flash column using a gradient of ethyl acetate in petroleum ether to give the title compound as yellow needles. Yield: 700 mg (61%).

MS (ES) MH⁺: 239 for C₇H₂NBrF₃O

¹H NMR (300 MHz, CDCl₃) δ: 7.8 (t, 1H) 10.2 (s, 1H).

Intermediate 43

### 5-Bromo-2-((2S,6R)-2,6-dimethyl-morpholin-4-yl)-3,4-difluoro-benzaldehyde

To an ice cooled and stirred solution of 5-Bromo-2,3,4-trifluoro-benzaldehyde (Intermediate 42, 250 mg, 1.05 mmol) in dry acetonitrile (2 mL), was added triethylamine (159 mg, 1.57 mmol) followed by 2,6-dimethylmorpholine (133 mg, 1.15 mmol), and the mixture heated at 80° C. for 12 hours. The reaction mixture was then cooled to room temperature and concentrated. The residue was dissolved in EtOAc (25 ml), washed with water (2×20 mL) followed by brine (2×20 mL), and dried over anhydrous sodium sulfate. The combined organic layeres were filtered and the filtrate evaporated under reduced pressure. The residue thus obtained was purified over a silica gel column using a gradient of ethyl acetate in petroleum ether to give the title product as a yellow solid. Yield: 220 mg (62%).

MS (ES) MH⁺: 336 for C₁₃H₁₄BrF₂NO₂

¹H NMR (300 MHz, CDCl₃) δ: 1.2 (d, 6H), 2.9-3.1 (m, 4H), 3.8 (m, 2H), 7.8 (dd, 1H), 10.2 (s, 1H).

Intermediate 44

### 2-((2R,6S)-2,6-Dimethylmorpholino)-3,4-difluoro-5((trimethylsilyl)ethynyl)benzaldehyde

To a stirred and degassed solution of 5-bromo-2-((2S,6R)-2,6-dimethyl-morpholin-4-yl)-3,4-difluoro-benzaldehyde (Intermediate 43, 2.0 g, 5.9 mmol) in dry DMF (10 mL), was added DIPEA (10 mL), copper iodide (56 mg, 0.29 mmol), dichloro-bis-(triphenyl phosphene)palladium (209 mg, 0.29 mmol) and trimethylsilyl acetylene (1.0 mL, 7.1 mmol), sequentially. The reaction mixture heated at 120° C. for 20 minutes under microwave conditions, cooled to room temperature, and concentrated. The residue thus obtained was purified over a silica gel column using a gradient of ethyl acetate in petroleum ether to give the title product as a yellow solid. Yield: 1.8 g.

MS (ES) MH⁺: 351 for C₁₈H₂₃F₂NO₂Si

¹H NMR (300 MHz, CDCl₃) δ: 0.26 (s, 9H), 1.2 (d, 6H), 3.05 (m, 4H), 3.89 (m, 1H), 7.7 (d, 1H), 10.1 (s, 1H).

Intermediates 45 and 46 were synthesized from the indicated starting materials using a method similar to the one described for the synthesis of Intermediate 44.

Intermediate 45

### 2-((2S,6R)-2,6-Dimethyl-morpholin-4-yl)-4-fluoro-5-trimethylsilanylethynyl-benzaldehyde

Starting material: 2-((2S,6R)-2,6-Dimethyl-morpholin-4-yl)-4-fluoro-5-iodo-benzaldehyde (Intermediate 31).

MS (ES) MH⁺: 334.2 for C₁₃H₁₅F₁INO₂

¹H NMR (300 MHz, CDCl₃) δ: 0.26 (s, 9H), 1.22 (d, 6H), 2.63 (t, 2H), 3.13 (d, 4H), 3.91 (m, 1H), 6.70 (d, 1H), 7.91(d, 1H), 10.05 (s, 1H).

Intermediate 46

### 2-((2S,6R)-2,6-Dimethyl-morpholin-4-yl)-3-fluoro-5-trimethylsilanylethynyl-benzaldehyde

Starting material: 2-((2S,6R)-2,6-Dimethyl-morpholin-4-yl)-3-fluoro-5-iodo-benzaldehyde (Intermediate 32).

MS (ES) MH⁺: 334.2 for C₁₈H₂₄FNO₂Si

¹H NMR (300 MHz, CDCl₃) δ: 0.2 (s, 9H), 1.1 (d, 6H), 2.8 (t, 2H), 3.1 (d, 1H), 3.75 (m, 2H), 7.6 (m, 2H), 10.2 (s, 1H).

Intermediate 47

### 2-[(2R,6S)-2,6-Dimethylmorpholin-4-yl]-5-ethynyl-3,4 difluorobenzaldehyde

To a solution of 2-((2R,6S)-2,6-dimethylmorpholino)-3,4-difluoro-5-((trimethylsilyl)ethynyl)benzaldehyde (Intermediate 44, 1.8 g, 5.1 mmol) in anhydrous MeOH (10 mL) was added potassium fluoride (0.29 g, 5.1 mmol). The reaction mixture was stirred at room temperature for 12 hours and concentrated. The residue thus obtained was purified over a silica gel column (230-400) using a gradient of ethylacetate in petroleum ether to give the title product as a yellow solid. Yield: 980 mg (70%).

MS (ES) MH⁺: 280 for C₁₅H₁₅F₂NO₂

¹H NMR (300 MHz, CDCl₃) δ: 1.2 (d, 6H) 3.1 (d, 4H) 3.3 (s, 1H), 3.8 (m, 2H), 7.7 (q, 1H), 10.2 (s, 1H).

Intermediates 48 and 49 were synthesized from the indicated starting materials using a method similar to the one described for the synthesis of Intermediate 47.

Intermediate 48

### 2-((2S,6R)-2,6-Dimethyl-morpholin-4-yl)-4-fluoro-5-trimethylsilanylethynyl-benzaldehyde

Starting material: 2-((2S,6R)-2,6-Dimethyl-morpholin-4-yl)-4-fluoro-5-trimethyl silanyl ethynyl-benzaldehyde (Intermediate 45).

MS (ES) MH⁺: 262 for C₁₃H₁₅F₁INO₂

¹H NMR (300 MHz, CDCl₃) δ: 1.2 (d, 6H), 2.65 (t, 2H), 3.1 (d, 4H), 3.3 (s, 1H), 3.9 (m, 2H), 6.7 (d, 1H), 7.9 (d, 1H), 10.1 (s, 1H).

Intermediate 49

### 2-((2S,6R)-2,6-Dimethyl-morpholin-4-yl)-5-ethynyl-3-fluoro-benzaldehyde

Starting material: 2-((2S,6R)-2,6-Dimethyl-morpholin-4-yl)-3-fluoro-5-trimethyl silanyl ethynyl-benzaldehyde (Intermediate 46).

MS (ES)MH⁺: 262.2 for C₁₅H₁₆FNO₂

¹H NMR (400 MHz, CDCl₃) δ: 1.1 (d, 6H), 2.9 (d, 2H), 3.1 (d, 2H), 3.8 (m, 2H), 4.3 (s, 1H), 7.55 (d, 1H), 7.6 (s, 1H), 10.2 (s, 1H).

Intermediate 50(a)

### 2-((2R,6S)-2,6-Dimethylmorpholino)-3,4-difluoro-5-((5-methyl-1,3,4-thiadiazol-2-yl)ethynyl)benzaldehyde

To a stirred and degassed solution of 2-[(2R,6S)-2,6-dimethylmorpholin-4-yl]-5-ethynyl-3,4 difluorobenzaldehyde (Intermediate 47, 0.35 mmol) in dry acetonitrile (5 mL) was added copper iodide (0.01 mmol), dichoro bis(triphenylphosphine)palladium (0.01 mmol), triethylamine (3.58 mmol), and 2-bromo-5-methyl-1,3,4-thiadiazole (77 mg, 0.43 mmol), sequentially. The reaction mixture was heated at 80° C. in a sealed tube for 12 hours, cooled to room temperature, filtered through a Celite pad, and concentrated. The residue was purified by flash chromatography over a silica gel column using a gradient of ethyl acetate in petroleum ether to give the title product. The product was taken for next step without further purification.

MS (ES) MH⁺: 378 for C₁₈H₁₇F₂N₃O₂S.

Intermediate 50(b)

### 2-[(2R,6S)-2,6-Dimethylmorpholin-4-yl]-3,4-difluoro-5-(1,3,4-thiadiazol-2-ylethynyl)benzaldehyde

To a stirred and degassed solution of 2-[(2R,6S)-2,6-dimethylmorpholin-4-yl]-5-ethynyl-3,4 difluorobenzaldehyde (Intermediate 47, 0.35 mmol) in dry DMF (2 mL) was added DIPEA (1.5 mL), copper iodide (0.01 mmol), dichorobis(triphenylphosphine) palladium (0.01 mmol), and 2-bromo-1,3,4-thiadiazole (0.71 mg, 0.43 mmol), sequentially. The reaction mixture was heated at 120° C. for 20 minutes in a microwave reactor, cooled to room temperature, and concentrated. The residue was purified by flash chromatography over a silica gel column using a gradient of ethyl acetate in petroleum ether to give the title product.

MS (ES) MH⁺: 364 for C₁₇H₁₅F₂N₃O₂S.

Intermediates 51 to 86 were synthesized from the indicated starting materials using a method similar to the ones described for the syntheses of Intermediates 50(a) and 50(b).

Intermediate 51

### 2-((2R,6S)-2,6-Dimethyl-morpholin-4-yl)-3,4-difluoro-5-thiazol-2-ylethynylbenzaldehyde

Starting materials: 2-[(2R,6S)-2,6-Dimethylmorpholin-4-yl]-5-ethynyl-3,4 difluorobenzaldehyde (Intermediate 47) and 2-bromothiazole.

MS(ES) MH⁺: for 363.4 C₁₈H₁₆F₂N₂O₂S

¹H NMR (400 MHz, CDCl₃) δ: 1.2 (d, 6H) 3.10 (q, 4H), 3.8 (m, 2H), 7.7 (q, 1H), 10.2 (s, 1H).

Intermediate 52

### 2-((2R,6S)-2,6-Dimethyl-morpholin-4-yl)-3,4-difluoro-5-thiazol-5-ylethynyl-benzaldehyde

Starting materials: 2-[(2R,6S)-2,6-Dimethylmorpholin-4-yl]-5-ethynyl-3,4 difluorobenzaldehyde (Intermediate 47) and 5-bromothiazole.

MS(ES) MH⁺: 363.2 for C₁₈H₁₆F₂N₂O₂S.

Intermediate 53

### 2-((2R,6S)-2,6-Dimethyl-morpholin-4-yl)-3,4-difluoro-5-thiophen-2-ylethynyl-benzaldehyde

Starting materials: 2-[(2R,6S)-2,6-Dimethylmorpholin-4-yl]-5-ethynyl-3,4 difluorobenzaldehyde (Intermediate 47) and 2-bromothiophene.

MS(ES) MH⁺: 362.2 for C₁₉H₁₇F₂NO₂S

¹H NMR (400 MHz, CDCl₃) δ: 1.2 (d, 6H) 3.1 (d, 4H), 3.8 (m, 2H), 7.04 (m, 1H), 7.3(m, 2H), 7.7 (q, 1H), 10.2 (s, 1H).

Intermediate 54

### 5-Benzo[b]thiophen-2-ylethynyl-2-((2R,6S)-2,6-dimethyl-morpholin-4-yl)-3,4-difluoro-benzaldehyde

Starting materials: 2-[(2R,6S)-2,6-Dimethylmorpholin-4-yl]-5-ethynyl-3,4 difluorobenzaldehyde (Intermediate 47) and 2-bromo-1,3-benzothiazole.

MS(ES) MH⁺: 412.2 for C₂₃H₁₉F₂NO₂S.

Intermediate 55

### 2-[(2R,6S)-2,6-Dimethylmorpholin-4-yl]-3,4-difluoro-5-[(1-methyl-1H-imidazol-2-yl)ethynyl]benzaldehyde

Starting materials: 2-[(2R,6S)-2,6-Dimethylmorpholin-4-yl]-5-ethynyl-3,4 difluorobenzaldehyde (Intermediate 47) and 2-bromo-1-methylimidazole.

MS(ES) MH⁺: 360.2 for C₁₉H₁₉F₂N₃O₂.

Intermediate 56

### 2-[(2R,6S)-2,6-Dimethylmorpholin-4-yl]-3,4-difluoro-5-[(1-methyl-1H-imidazol-4-yl)ethynyl]benzaldehyde

Starting materials: 2-[(2R,6S)-2,6-Dimethylmorpholin-4-yl]-5-ethynyl-3,4 difluorobenzaldehyde (Intermediate 47) and 4-bromo-1-methylimidazole.

MS(ES) MH⁺: 360.2 for C₁₉H₁₉F₂N₃O₂.

Intermediate 57

### 2-[(2R,6S)-2,6-Dimethylmorpholin-4-yl]-3,4-difluoro-5-{[5-(1H-tetrazol-5-yl)thiophen-2-yl]ethynyl}benzaldehyde

Starting materials: 2-[(2R,6S)-2,6-Dimethylmorpholin-4-yl]-5-ethynyl-3,4 difluorobenzaldehyde (Intermediate 47) and 4-bromo-1-methylimidazole.

MS(ES) MH⁺: 430.2 for C₂₀H₁₇F₂N₅O₂S.

Intermediate 58

### 2-[(2R,6S)-2,6-Dimethylmorpholin-4-yl]-3,4-difluoro-5-(1H-imidazol-4-ylethynyl)benzaldehyde

Starting materials: 2-[(2R,6S)-2,6-Dimethylmorpholin-4-yl]-5-ethynyl-3,4 difluorobenzaldehyde (Intermediate 47) and 4-bromo-1-methylimidazole.

MS(ES) MH⁺: 346.0 for C₁₈H₁₇F₂N₃O₂.

Intermediate 59

### 2-[(2R,6S)-2,6-Dimethylmorpholin-4-yl]-3,4-difluoro-5-(1H-imidazol-2-ylethynyl)benzaldehyde

Starting materials: 2-[(2R,6S)-2,6-Dimethylmorpholin-4-yl]-5-ethynyl-3,4 difluorobenzaldehyde (Intermediate 47) and 4-bromo-1-methylimidazole.

MS(ES) MH⁺: 346.2 for C₁₈H₁₇F₂N₃O₂.

Intermediate 60

### 2-[(2R,6S)-2,6-Dimethylmorpholin-4-yl]-3,4-difluoro-5-{[5-(1H-pyrazol-5-yl)thiophen-2-yl]ethynyl}benzaldehyde

Starting materials: 2-[(2R,6S)-2,6-Dimethylmorpholin-4-yl]-5-ethynyl-3,4 difluorobenzaldehyde (Intermediate 47) and 4-bromo-1-methylimidazole.

MS(ES) MH⁺: 428.0 for C₂₂H₁₉F₂N₃O₂S.

Intermediate 61

### 2-[(2R,6S)-2,6-Dimethylmorpholin-4-yl]-3,4-difluoro-5-(pyridin-3-ylethynyl)benzaldehyde

Starting materials: 2-[(2R,6S)-2,6-Dimethylmorpholin-4-yl]-5-ethynyl-3,4 difluorobenzaldehyde (Intermediate 47) and 3-bromopyridine.

MS(ES) MH⁺: 357.4 for C₂₀H₁₈F₂N₂O₂.

Intermediate 62

### 2-[(2R,6S)-2,6-Dimethylmorpholin-4-yl]-3,4-difluoro-5-(pyrimidin-2-ylethynyl)benzaldehyde

Starting materials: 2-[(2R,6S)-2,6-Dimethylmorpholin-4-yl]-5-ethynyl-3,4 difluorobenzaldehyde (Intermediate 47) and 2-bromopyrimidine.

¹H NMR (400 MHz, CDCl₃) δ: 1.2 (d, 6H) 3.1(m, 4H), 3.8 (m, 2H),7.3(m, 1H), 7.9 (q, 1H), 8.8(d, 2H), 10.1 (s, 1H).

Intermediate 63

### 2-[(2R,6S)-2,6-Dimethylmorpholin-4-yl]-3,4-difluoro-5-(pyrazin-2-ylethynyl)benzaldehyde

Starting materials: 2-[(2R,6S)-2,6-Dimethylmorpholin-4-yl]-5-ethynyl-3,4 difluorobenzaldehyde (Intermediate 47) and 2-bromopyrazine

¹H NMR (400 MHz, CDCl₃) δ: 1.2 (d, 6H) 3.1 (q, 4H), 3.8 (m, 2H), 7.8 (q, 1H),8.5 (d, 1H), 8.6(q, 1H), 8.68 (s, 2H), 8.8 (d, 1H), 9.6 (s, 1H),10.1 (s, 1H).

Intermediate 64

### 2-[(2R,6S)-2,6-Dimethylmorpholin-4-yl]-4-fluoro-5-(1,3,4-thiadiazol-2-ylethynyl)benzaldehyde

Starting materials: 2-((2S,6R)-2,6-Dimethyl-morpholin-4-yl)-5-ethynyl-4-fluoro-benzaldehyde (Intermediate 48) and 2-bromo-[1,3,4]thiadiazole.

MS (ES) MH⁺: 346.2 for C₁₇H₁₆FN₃O₂S.

Intermediate 65

### 2-[(2R,6S)-2,6-Dimethylmorpholin-4-yl]-4-fluoro-5-[(5-methyl-1,3,4-thiadiazol-2-yl)ethynyl]benzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromo-5-methyl-1,3,4-thiadiazole.

MS (ES) MH⁺: 360.2 for C₁₈H₁₈FN₃O₂S.

Intermediate 66

### 2-[(2R,6S)-2,6-Dimethylmorpholin-4-yl]-4-fluoro-5-(1,3-thiazol-2-ylethynyl)benzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromothiazole.

MS (ESP) MH⁺: 345.2 for C₁₈H₁₇FN₂O₂S.

Intermediate 67

### 2-[(2R,6S)-2,6-Dimethylmorpholin-4-yl]-4-fluoro-5-(1,3-thiazol-5-ylethynyl)benzaldehyde

Starting materials: 2-((2S,6R)-2,6-Dimethyl-morpholin-4-yl)-4-fluoro-5-trimethylsilanylethynyl-benzaldehyde (Intermediate 48) and 5-bromothiazole.

MS (ES) MH⁺: 345.2 for C₁₈H₁₇FN₂O₂S.

Intermediate 68

### 2-[(2R,6S)-2,6-Dimethyl morpholin-4-yl]-4-fluoro-5-(thiophen-2-ylethynyl)benzaldehyde

Starting materials: 2-((2S,6R)-2,6-Dimethyl-morpholin-4-yl)-4-fluoro-5-trimethylsilanylethynyl-benzaldehyde and (Intermediate 48) and 2-bromothiophene.

MS (ES) MH⁺: 344.2 for C₁₉H₁₈FNO₂S.

Intermediate 69

### 5-(1-Benzothiophen-2-ylethynyl)-2-[(2R,6S)-2,6-dimethylmorpholin-4-yl]-4-fluorobenzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromo-1,3-benzothiazole.

MS (ES) MH⁺: 394.0 for C₂₃H₂₀FNO₂S

Intermediate 70

### 2-[(2R,6S)-2,6-Dimethylmorpholin-4-yl]-4-fluoro-5-[(1-methyl-1H-imidazol-2-yl)ethynyl]benzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromo-1-methylimidazole.

MS (ES) MH⁺: 342.2 for C₁₉H₂₀FN₃O₂.

Intermediate 71

### 5-(1,3-Benzothiazol-2-ylethynyl)-2-[(2R,6S)-2,6-dimethylmorpholin-4-yl]-3-fluorobenzaldehyde

Starting materials: 2-((2S,6R)-2,6-Dimethyl-morpholin-4-yl)-5-ethynyl-3-fluoro-benzaldehyde (Intermediate 49) and 2-bromobenzthiazole.

MS (ES) MH⁺: 395.2 for C₂₂H₁₉FN₂O₂S

¹H NMR (400 MHz, CDCl₃) δ: 1.22 (t, 6H), 3.08(m, 4H), 3.86(m, 2H), 7.46-7.57 (m, 3H), 7.77(m, 2H), 8.09(d, 1H), 10.30(s, 1H).

Intermediate 72

### 2-[(2R,6S)-2,6-Dimethylmorpholin-4-yl]-4-fluoro-5-(pyrazin-2-ylethynyl)benzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromopyrazine.

MS (ES) MH⁺: 340.0 for C₁₉H₁₈FN₃O₂.

Intermediate 73

### 2-[(2R,6S)-2,6-Dimethylmorpholin-4-yl]-4-fluoro-5-(pyridin-3-ylethynyl)benzaldehyde

Starting materials: 2-((2S,6R)-2,6-Dimethyl-morpholin-4-yl)-4-fluoro-5-trimethylsilanylethynyl-benzaldehyde (Intermediate 48) and 3-bromopyridine.

MS (ES) MH⁺: 339.2 for C₂₀H₁₉FN₂O₂.

Intermediate 74

### 2-[(2R,6S)-2,6-Dimethylmorpholin-4-yl]-4-fluoro-5-(pyrimidin-2-ylethynyl)benzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromopyrimidine.

MS (ES) MH⁺: 340.2 for C₁₉H₁₈FN₃O₂.

Intermediate 75

### 5-(1,3-Benzothiazol-2-ylethynyl)-2-[(2R,6S)-2,6-dimethylmorpholin-4-yl]-4-fluorobenzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromobenzthiazole.

MS (ES) MH⁺: 395.2 for C₂₂H₁₉FN₂O₂S

¹H NMR (400 MHz, CDCl₃) δ: 1.22 (t, 6H), 3.08(m, 4H), 3.86(m, 2H), 7.46-7.57 (m, 3H), 7.77(m, 2H), 8.09(d, 1H), 10.30(s, 1H).

Intermediate 76

### 2-[(2R,6S)-2,6-Dimethylmorpholin-4-yl]-3-fluoro-5-(1,3,4-thiadiazol-2-ylethynyl)benzaldehyde

Starting materials: 2-((2S,6R)-2,6-Dimethyl-morpholin-4-yl)-5-ethynyl-3-fluoro-benzaldehyde (Intermediate 49) and 2-bromo-[1,3,4]thiadiazole.

MS (ES) MH⁺: 346.2 for C₁₇H₁₆FN₃O₂S.

Intermediate 77

### 2-[(2R,6S)-2,6-Dimethylmorpholin-4-yl]-3-fluoro-5-[(5-methyl-1,3,4-thiadiazol-2-yl)ethynyl]benzaldehyde

Starting materials: 2-((2S,6R)-2,6-Dimethyl-morpholin-4-yl)-5-ethynyl-3-fluoro-benzaldehyde (Intermediate 49) and 2-bromo-5-methyl-1,3,4-thiadiazole.

MS (ES) MH⁺: 360.2 for C₁₈H₁₈FN₃O₂S.

Intermediate 78

### 2-[(2R,6S)-2,6-Dimethylmorpholin-4-yl]-3-fluoro-5-(1,3-thiazol-2-ylethynyl)benzaldehyde

Starting materials: 2-((2S,6R)-2,6-Dimethyl-morpholin-4-yl)-5-ethynyl-3-fluoro-benzaldehyde (Intermediate 49) and 2-bromothiazole.

MS (ESP) MH⁺: 345.2 for C₁₈H₁₇FN₂O₂S.

Intermediate 79

### 2-[(2R,6S)-2,6-Dimethylmorpholin-4-yl]-3-fluoro-5-(1,3-thiazol-5-ylethynyl)benzaldehyde

Starting materials: 2-((2S,6R)-2,6-Dimethyl-morpholin-4-yl)-5-ethynyl-3-fluoro-benzaldehyde

(Intermediate 49) and 5-bromothiazole.

MS (ES) MH⁺: 345.2 for C₁₈H₁₇FN₂O₂S.

Intermediate 80

### 2-[(2R,6S)-2,6-Dimethylmorpholin-4-yl]-3-fluoro-5-(thiophen-2-ylethynyl)benzaldehyde

Starting materials: 2-((2S,6R)-2,6-Dimethyl-morpholin-4-yl)-5-ethynyl-3-fluoro-benzaldehyde

(Intermediate 49) and 2-bromothiophene.

MS (ES) MH⁺: 344.2 for C₁₉H₁₈FNO₂S.

Intermediate 81

### 5-(1-Benzothiophen-2-ylethynyl)-2-[(2R,6S)-2,6-dimethylmorpholin-4-yl]-3-fluorobenzaldehyde

Starting materials: 2-((2S,6R)-2,6-Dimethyl-morpholin-4-yl)-5-ethynyl-3-fluoro-benzaldehyde

(Intermediate 49) and 2-bromobenzthiophene.

MS (ES) MH⁺: 394.0 for C₂₃H₂₀FNO₂S.

Intermediate 82

### 2-[((2R,6S)-2,6-Dimethylmorpholin-4-yl]-3-fluoro-5-[(1-methyl-1-H-imidazol-2-yl)ethynyl]benzaldehyde

Starting materials: 2-((2S,6R)-2,6-Dimethyl-morpholin-4-yl)-5-ethynyl-3-fluoro-benzaldehyde

(Intermediate 49) and 2-bromo-1-methylimidazole.

MS (ES) MH⁺: 342.2 for C₁₉H₂₀FN₃O₂.

Intermediate 83

### 5-(1,3-benzoxazol-2-ylethynyl)-2-[(2R,6S)-2,6-dimethylmorpholin-4-yl]-4-fluorobenzaldehyde

Starting materials: 2-((2S,6R)-2,6-Dimethyl-morpholin-4-yl)-4-fluoro-5-trimethylsilanylethynyl-benzaldehyde (Intermediate 48) and 2-bromobenzoxazole.

MS (ESP) MH⁺: 379.2 for C₂₂H₁₉FN₂O₃.

Intermediate 84

### 2-[(2R,6S)-2,6-Dimethylmorpholin-4-yl]-3-fluoro-5-(pyrazin-2-ylethynyl)benzaldehyde

Starting materials: 2-((2S,6R)-2,6-Dimethyl-morpholin-4-yl)-5-ethynyl-3-fluoro-benzaldehyde

(Intermediate 49) and 2-bromopyrazine.

MS (ES) MH⁺: 340.0 for C₁₉H₁₈FN₃O₂.

Intermediate 85

### 2-[(2R,6S)-2,6-Dimethylmorpholin-4-yl]-3-fluoro-5-(pyridin-3-ylethynyl)benzaldehyde

Starting materials: 2-((2S,6R)-2,6-Dimethyl-morpholin-4-yl)-5-ethynyl-3-fluoro-benzaldehyde

(Intermediate 49) and 3-bromopyridine.

MS (ES) MH⁺: 339.2 for C₂₀H₁₉FN₂O₂.

Intermediate 86

### 2-[(2R,6S)-2,6-Dimethylmorpholin-4-yl]-3-fluoro-5-(pyrimidin-2-ylethynyl)benzaldehyde

Starting materials: 2-((2S,6R)-2,6-Dimethyl-morpholin-4-yl)-5-ethynyl-3-fluoro-benzaldehyde

(Intermediate 49) and 2-bromopyrimidine.

MS (ES) MH⁺: 340.2 for C₁₉H₁₈FN₃O₂.

Intermediate 87

### 2,3,4-Trifluoro-5-iodobenzaldehyde

To a solution of diisopropylamine (4.3 g, 42.6 mmol) in THF (50 ml) was added n-butyl lithium (21.3 ml, 2 N in hexane) dropwise at −10° C., and the solution was stirred for 30 minutes at −10° C. The reaction mixture was cooled to −78° C. and to this was added 1,2,3-trifluoro-4-iodobenzene (5 g, 19.38 mmol) in THF (50 ml), stirred at −78° C. under a nitrogen atmosphere. After stirring for 5 hours at this temperature, DMF (7 ml, 89 mmol) was added dropwise such that the temperature was maintained below −60° C. The reaction mixture was slowly allowed to warm to room temperature and stirred overnight. The reaction mixture was treated with saturated aqueous NH₄Cl solution and the aqueous layer extracted with ethyl acetate (2×100 ml). The organic phases were combined, dried over anhydrous sodium sulfate, and concentrated. The residue was purified over silica gel flash column using a gradient of ethyl acetate in petroleum ether to give the title product as a yellow solid. Yield: 2.4 g (45%).

MS (MH⁺): 287.2 for C₇H₂F₃IO

¹H NMR (300 MHz, CDCl₃) δ: 8.1 (m, 1H), 10.2 (s, 1H).

Intermediate 88

### 2-[(2R,6S)-2,6-Dimethylmorpholin-4-yl]-3,4-difluoro-5-iodobenzaldehyde

To an ice cooled and stirred solution of 2,3,4-trifluoro-5-iodobenzaldehyde (Intermediate 87, 2.4 g, 8.39 mmol) in dry acetonitrile (25 ml), was added triethylamine (1.32 ml, 12.59 mmol), followed by 2,6-dimethylmorpholine (1.06 g, 9.23 mmol). The reaction mixture was heated at 80° C. for 12 hours, cooled to room temperature, and concentrated. The residue was dissolved in ethyl acetate (200 ml), washed with water (2×50 ml) and brine (50 ml), dried over anhydrous sodium sulfate, and concentrated. The residue was purified over silica gel column using a gradient of ethyl acetate in petroleum ether to give the title product as a yellow solid. Yield: 2.8 g (87%) MS (MH⁻): 382.2 for C₁₃H₁₄F₂INO₂

¹H NMR (300MHz, CDCl₃) δ: 1.2 (d, 6H), 3.1 (m, 4H), 3.8 (m, 2H), 8.0 (m, 1H), 10.2 (s, 1H).

Intermediate 89

### (2R,4S,4aS)-rel-9,10-Difluoro-8-iodo-2,4-dimethyl-2,4,4a,6-tetrahydro-1H,1′H-spiro[[1,4]oxazino[4,3-a]quinoline-5,5′-pyrimidine]-2′,4′,6′(3′H)-trione

To a stirred solution of 2-[(2R,6S)-2,6-dimethylmorpholin-4-yl]-3,4-difluoro-5-iodobenzaldehyde (Intermediate 88, 2.0 g, 5.2 mmol) in dry IPA (30 ml) was added barbituric acid (0.75 g, 5.7 mmol), and the reaction mixture was stirred for 14 hours at 80° C., under a nitrogen atmosphere. The IPA was removed under vacuum and the residue was subjected to silica gel column chromatography using a gradient of ethyl acetate in petroleum ether to give the title product in the form of a white solid. Yield: 2.0 g (80%)

MS (MH⁺): 492.2 for C₁₇H₁₆F₂IN₃O₄

¹H NMR (400 MHz, DMSO-d₆) δ: 0.9 (d, 3H), 1.1 (d, 3H), 2.8 (d, 1H), 3.0 (m, 1H), 3.4 (d, 1H), 3.6 (m, 1H), 3.7 (m, 1H), 3.8 (d, 1H), 4.0 (d, 1H), 7.1 (d, 1H), 11.5 (s, 1H), 11.8 (s, 1H).

The (2S,4R,4aR) and (2R,4S,4aS) enantiomers of the title compound were separated by Supercritical Fluid Chromatography using a Chiralcel OJ-H, 21×250mm, 5μ column (6 minute elution with 30% MeOH, 70% CO₂ at 60 ml/min, 40° C., and 100 bar with detection at 220nm).

Intermediate 89(a), First Eluting Compound

### (2S,4R,4aR)-9,10-Difluoro-8-iodo-2,4-dimethyl-2,4,4a,6-tetrahydro-1H,1′H-spiro[[1,4]oxazino[4,3-a]quinoline-5,5′-pyrimidine]-2′,4′,6′(3′H)-trione

MS (MH⁺): 492.2 for C₁₇H₁₆F₂IN₃O₄

¹H NMR (400 MHz, DMSO-d₆) δ: 0.9 (d, 3H), 1.1 (d, 3H), 2.8 (d, 1H), 3.0 (m, 1H), 3.4 (d, 1H), 3.6 (m, 1H), 3.7 (m, 1H), 3.8 (d, 1H), 4.0 (d, 1H), 7.1 (d, 1H), 11.65 (s, broad, 2H). 100% ee by chiral HPLC.

[∝]=+251 (c=0.1 in methanol).

Intermediate 89(b), Second Eluting Compound

### (2R,4S,4aS)-9,10-Difluoro-8-iodo-2,4-dimethyl-2,4,4a,6-tetrahydro-1H,1′H-spiro[[1,4]oxazino[4,3-a]quinoline-5,5′-pyrimidine]-2′,4′,6′(3′H)-trione

MS (MH⁺): 492.2 for C₁₇H₁₆F₂IN₃O₄

¹H NMR (400 MHz, DMSO-d₆) δ: 0.9 (d, 3H), 1.1 (d, 3H), 2.8 (d, 1H), 3.0 (m, 1H), 3.4 (d, 1H), 3.6 (m, 1H), 3.7 (m, 1H), 3.8 (d, 1H), 4.0 (d, 1H), 7.1 (d, 1H), 11.65 (s, broad, 2H). 98% ee by chiral HPLC.

[∝]=−216 (c=0.1 in methanol).

### Example 1

### (2R,4S,4aS)-rel-2,4-Dimethyl-8-((trimethylsilypethynyl)-2,4,4a,6-tetrahydro-1H,1′H-spiro[[1,4]oxazino[4,3-a]quinoline-5,5′-pyrimidine]-2′,4′,6′(3′H)-trion

To a reaction vial containing (2R,4S,4aS)-8-bromo-2,4-dimethyl-2,4,4a,6-tetrahydro-1H,1′H-spiro[[1,4]oxazino[4,3-a]quinoline-5,5′-pyrimidine]-2′,4′,6′(3′H)-trione (Intermediate 40, 150 mg, 0.37 mmol) and copper(I) iodide (0.831 μL, 0.02 mmol) at room temperature was added Tris(dibenzylideneacetone)dipalladium (0) (33.6 mg, 0.04 mmol) under an Argon atmosphere. Purging with argon was continued for 20 min. Dioxane (5 mL) was added and argon was bubbled through 20 min. After de-aerating by bubbling N₂ through, triethylamine (0.205 mL, 1.47 mmol) was added via syringe. Tri-t-butylphosphine (0.219 mL, 0.07 mmol) and trimethylsilylacetylene (0.103 mL, 0.73 mmol) were added via syringe. The mixture was stirred at room temperature under Ar overnight. The mixture was worked up by diluting with EtOAc and filtering through a pad of silica gel, rinsing through with EtOAc. After removal of solvent, the residue was chromatographed on silica gel (100% CH₂Cl₂ followed by gradient elution to 30% EtOAc in CH₂Cl₂). The product from the chromatography was taken up in MeOH. Solids precipitated to afford 32 mg of the title product as a solid.

MS (ES) MH⁺: 426 for C₂₂H₂₇N₃O₄Si

¹H NMR (DMSO-d₆): 0.9 (d, 3H), 1.1 (d, 3H), 2.7-2.9 (m, 2H), 3.2 (m, 1H), 3.4-3.7 (m, 3H), 4.0 (d, 1H), 6.8 (d, 1H), 7.0 (s, 1H), 7.2 (d, 1H), 11.5 (s, 1H), 11.8 (s, 1H).

### Example 2

### (2R,4S)-rel-2,4-Dimethyl-8-(pyridin-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

To a solution of (2R,4S,4aS)-8-iodo-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione (Intermediate 41, 460 mg, 1 mmol) in dry acetonitrile (2 mL), was added PdCl₂(PPh₃)₂ (0.05 mmol), CuI (0.05 mmol), Et₃N (7.9 mmol) and 2-ethynyl pyridine (103 mg, 1 mmol), sequentially. The reaction mixture was heated at 85° C. in a sealed tube for 12 hours, cooled to room temperature, filtered through Celite pad, and concentrated. The residue thus obtained was purified over flash chromatography over silica gel column using a gradient of ethyl acetate in petroleum ether to give the title compound as a solid.

MS(ES) MH⁺: 431 for C₂₄H₂₂N₄O₄

¹H NMR (400 MHz, CD₃OD) δ: 1.1 (d, 3H), 1.3 (d, 3H), 3.0 (m, 1H), 3.1 (d, 1H), 3.3 (d, 1H), 3.70-3.76 (m, 2H), 3.9 (d, 1H), 4.2 (d, 1H), 6.9 (d, 1H), 7.18 (s, 1H), 7.35-7.40 (m, 2H), 7.6 (d, 1H), 7.85 (m, 1H), 8.51 (d, 1H).

Examples 3 and 4 were synthesized from (2R,4S,4aS)-8-iodo-2,4-dimethyl-1,2,4,4a-tetrahydro-2′H,6H-spiro[1,4-oxazino[4,3-c]quinoline-5,5′-pyrimidine]-2′,4′,6′(1′H,3′H)-trione (Intermediate 41) and the indicated starting materials using a method similar to the one described for the synthesis of Example 2.

### Example 3

### (2R,4S,4aS)-rel-2,4-Dimethyl-8-(pyridin-4-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 4-Ethynyl pyridine.

MS(ES) MH⁺:431 for C₂₄H₂₂N₄O₄

¹H NMR (400 MHz, CD₃OD) δ: 1.1 (d,3H), 1.3 (d, 3H), 3.0 (m, 1H), 3.1 (d,1H), 3.25 (d, 1H), 3.7 (m, 3H), 3.9 (d, 1H), 4.2 (d, 1H), 6.9 (d, 1H), 7.2 (s, 1H), 7.4 (d, 1H), 7.5 (s, 2H), 8.5 (brs, 3H).

### Example 4

### (2R,4S,4aS)-rel-2,4-Dimethyl-8-[(1-methyl-1H-imidazol-2-yl)ethynyl]-1,2,4,4a-tetrahydro-2′H,6H-spiro[1,4-oxazino[4,3-a]quinoline-5,5′-pyrimidine]-2′,4′,6′(1′H,3′H)-trione

Starting material: 1-Methyl-2-ethynyl-imidazole.

MS(ES) MH⁺: 434 for C₂₃H₂₃N₅O₄

¹H NMR (CD₃OD) δ: 1.1 (d, 3H), 1.3 (d, 3H), 2.95 (m,1H), 3.1 (d, 1H), 3.3 (d, 1H), 3.7 (m, 1H), 3.74-3.76 (m, 4H), 3.92 (d, 1H), 4.14 (d, 1H), 6.87 (d, 1H), 7.11 (s, 1H), 7.2 (s,1H), 7.3 (d, 1H), 7.69 (s, 1H).

### Example 5

### (2S,4R,4aR)-rel-9,10-Difluoro-2,4-dimethyl-8-[(5-methyl-1,3,4-thiadiazol-2-yl)ethynyl]-1,2,4,4a-tetrahydro-2′H,6H-spiro[1,4-oxazino[4,3-a]quinoline-5,5′-pyrimidine]-2′,4′,6′(1′H,3′H)-trione

To a stirred solution of 2-((2R,6S)-2,6-dimethylmorpholino)-3,4-difluoro-5-((5-methyl-1,3,4-thiadiazol-2-yl)ethynyl)benzaldehyde (Intermediate 50(a), 150 mg, 0.52 mmol) in dry IPA (5 mL) was added barbituric acid (66 mg, 0.52 mmol) and the solution heated around 80° C. for 12 h. Solvents were evaporated and the residue thus obtained purified over silica gel-column to give the title product as a solid.

MS (ES) MH⁺: 488 for C₂₂H₁₉F₂N₅O₄S

¹H NMR (300 MHz, DMSO-d₆) δ: 0.9 (d, 3H), 1.1 (d, 3H), 2.9 (d, 1H), 3.1 (m, 1H), 3.5 (d, 1H), 3.6 (m, 1H), 3.7 (m, 1H), 3.9 (d, 1H), 4.1 (d, 1H), 7.1 (d, 1H), 11.6 (s, 1H), 11.9 (s, 1H).

Examples 6 to 46 were synthesized from pyrimidine-2,4,6(1H,3H,5H)-trione and the indicated starting materials using a method similar to the one described for the synthesis of Example 5.

### Example 6

### (2S,4R,4aR)-rel-9,10-Difluoro-2,4-dimethyl-8-[(trimethylsilyl)ethynyl]-1,2,4,4a-tetrahydro2′H,6H-spiro[1,4-oxazino[4,3-a]quinoline-5,5′-pyrimidine]-2′,4′,6′(1′H,3′H)-trione

Starting material: 2-((2R,6S)-2,6-Dimethylmorpholino)-3,4-difluoro-5-((trimethylsilyl)ethynyl)benzaldehyde (Intermediate 44).

MS (ES) M+H⁻: 462 for C₂₂H₂₅F₂N₃O₄Si

¹H NMR (400 MHz, DMSO-d₆) δ: 0.2 (d, 9H), 0.9 (d, 3H), 1.1 (d, 3H), 2.8 (d, 1H), 3.05 (m, 1H), 3.4 (d, 1H), 3.6 (m, 1H), 3.7 (m,1H), 3.8 (d, 1H), 4.0 (d, 1H), 7.0 (d, 1H), 8.2 (s, 1H), 9.15 (s, 1H), 11.5 (s, 1H), 11.9 (s, 1H).

### Example 7

### (2S,4R,4aR)-rel-9,10-Difluoro-2,4-dimethyl-8-(1,3,4-thiadiazol-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-(1,3,4-thiadiazol-2-ylethynyl)benzaldehyde (Intermediate 50(b)).

MS (ES) M+H⁻: 474 for C₂₃H₁₉F₂N₃O₄S

¹H NMR (400 MHz, DMSO-d₆) δ: 0.9 (d, 3H), 1.1 (d, 3H), 2.9 (d, 1H), 3.1 (m, 1H), 3.5 (d, 1H), 3.65 (m, 1H), 3.8 (m, 1H), 3.9 (d, 1H), 4.1 (d, 1H), 7.1 (d, 1H), 9.7 (s, 2H), 11.6 (s, 1H), 11.9.

### Example 8

### (2S,4R,4aR)-rel-9,10-Difluoro-2,4-dimethyl-8-(1,3-thiazol-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-((2R,6S)-2,6-Dimethyl-morpholin-4-yl)-3,4-difluoro-5-thiazol-2-ylethynylbenzaldehyde (Intermediate 51).

MS (ES) M+H⁺: 473 for C₂₂H₁₈F₂N₄O₄S

¹H NMR (400 MHz, DMSO-d₆) δ: 0.9 (d, 3H), 1.1 (d, 3H), 2.9 (d, 1H), 3.1 (m, 1H), 3.5 (d, 1H), 3.6 (m, 1H), 3.75 (m, 1H), 3.9 (d, 1H), 4.1 (d, 1H), 7.1 (d, 1H), 7.9 (d, 1H), 7.9 (d, 1H), 11.7 (bs, 2H).

### Example 9

### (2S,4R,4aR)-rel-9,10-Difluoro-2,4-dimethyl-8-(1,3-thiazol-5-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-((2R,6S)-2,6-Dimethyl-morpholin-4-yl)-3,4-difluoro-5-thiazol-5-ylethynyl-benzaldehyde (Intermediate 52).

MS (ES) M+H⁻: 473 for C₂₂H₁₈F₂N₄O₄S

¹H NMR (400 MHz, DMSO-d₆) δ: 0.9 (d, 3H), 1.1 (d, 3H), 2.9 (d, 1H), 3.1 (m, 1H), 3.4 (d, 1H), 3.6 (m, 1H), 3.7 (m, 1H), 3.9 (d, 1H), 4.05 (d, 1H), 7.0 (d, 1H), 8.2 (s, 1H), 9.15 (s, 1H), 11.5 (s, 1H), 11.9 (s, 1H).

### Example 10

### (2S,4R,4aR)-rel-9,10-Difluoro-2,4-dimethyl-8-(thiophen-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-((2R,6S)-2,6-Dimethyl-morpholin-4-yl)-3,4-difluoro-5-thiophen-2-ylethynyl-benzaldehyde (Intermediate 53).

MS (ES) M+H⁻: 472 for C₂₃H₁₉F₂N₃O₄S ¹H NMR (400 MHz, DMSO-d₆) δ: 0.9 (d, 3H), 1.1 (d, 3H), 2.9 (d, 1H), 3.1 (m, 1H), 3.4 (d, 1H), 3.6 (m, 1H), 3.75 (m,1H), 3.9 (d,1H), 4.0 (d, 1H), 7.0 (d, 1H), 7.1 (m, 1H), 7.4 (d, 1H), 7.7 (d, 1H), 11.5 (s, 1H), 11.9 (s, 1H).

### Example 11

### (2S,4R,4aR)-rel-8-(1-Benzothiophen-2-ylethynyl)-9,10-difluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 5-Benzo[b]thiophen-2-ylethynyl-2-((2R,65)-2,6-dimethyl-morpholin-4-yl)-3,4-difluoro-benzaldehyde (Intermediate 54).

MS (ES) M+H⁻: 522 for C₂₇H₂₁F₂N₃O₄S

¹H NMR (400 MHz, DMSO-d₆) δ: 0.9 (d, 3H), 1.1 (d, 3H), 2.9 (d, 1H), 3.1 (m, 1H), 3.45 (d, 1H), 3.65 (m, 1H), 3.7 (m,1H), 3.9 (d,1H), 4.1 (d, 1H), 7.0 (d, 1H), 7.45 (m, 2H), 7.7 (s, 1H), 7.9 (m, 1H), 8.0 (m, 1H), 11.55 (s, 1H), 11.9 (s, 1H).

### Example 12

### (2R,4S,4aS)-rel-9,10-Difluoro-2,4-dimethyl-8-[(1-methyl-1H-imidazol-2-yl)ethynyl]-1,2,4,4a-tetrahydro-2′H,6H-spiro[1,4-oxazino≡[4,3-a]quinoline-5,5′-pyrimidine]-2′,4′,6′(1′H,3H)-trione

Starting material: 2-[(2R,6S)-2,6-Dimethylmorpholin-4-yl]-3,4-difluoro-5-[(1-methyl-1H-imidazol-2-yl)ethynyl]benzaldehyde (Intermediate 55).

MS (ES) M+H⁻: 470 for C₂₃H₂₁F₂N₅O

¹H NMR (400 MHz, DMSO-d₆) δ: 0.9 (d, 3H), 1.1 (d, 3H), 2.9 (d, 1H), 3.1 (m, 1H), 3.5 (d, 1H), 3.66 (m, 1H), 3.7 (s, 3H), 3.8 (m, 1H), 3.9 (d, 1H), 4.1 (d, 1H), 7.0 (s, 1H), 7.04 (s, 1H), 7.3 (s, 1H), 11.55 (s, 1H), 11.9 (s, 1H).

### Example 13

### (2S,4R,4aR)-rel-9,10-Difluoro-2,4-dimethyl-8-[(1-methyl-1H-imidazol-4-yl)ethynyl]-1,2,4,4a-tetrahydro-2′H,6H-spiro[1,4-oxazino[4,3-a]quinoline-5,5′-pyrimidine]-2′,4′,6′-(1′H,3′H)-trione

Starting material: 2-[(2R,6S)-2,6-Dimethylmorpholin-4-yl]-3,4-difluoro-5-[(1-methyl-1H-imidazol-4-yl)ethynyl]benzaldehyde (Intermediate 56).

MS (ES) M+H⁻: 470 for C₂₃H₂₁F₂N₅O₄

¹H NMR (400 MHz, DMSO-d₆) δ: 0.9 (d, 3H), 1.1 (d, 3H), 2.9 (d, 1H), 3.05 (m, 1H), 3.4 (d, 1H), 3.6 (s, 3H), 3.6 (m, 1H), 3.8 (d, 1H), 4.0 (d, 1H), 6.9 (d, 1H), 7.5 (bs, 1H), 7.65 (bs, 1H), 11.5 (s, 1H), 11.8 (s, 1H).

### Example 14

### (2R,4S,4aS)-rel-9,10-Difluoro-2,4-dimethyl-8-{[5-(1H-tetrazol-5-yl)thiophen-2-yl]ethynyl}-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-{[5-(1H-tetrazol-5-yl)thiophen-2-yl]ethynyl}benzaldehyde (Intermediate 57).

MS (ES) M+H⁻: 540 for C₂₄H₁₉F₂N₇O₄S

¹H NMR (400 MHz, DMSO-d₆) δ: 0.9 (d, 3H), 1.1 (d, 3H), 2.9 (d, 1H), 3.1 (m, 1H), 3.5 (d, 1H), 3.7 (m, 1H), 3.75 (m, 1H), 3.9 (d, 1H), 4.1 (d, 1H), 7.0 (d, 1H), 7.3 (d, 1H), 7.4 (d, 1H), 11.6 (s, 1H), 11.9 (s, 1H).

Example 15

### (2S,4R,4aR)-rel-9,10-Difluoro-8-(1H-imidazol-4-ylethynyl)-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-(1H-imidazol-4-ylethynyl)benzaldehyde (Intermediate 58).

MS (ES) M+H⁻: 456 for C₂₂H₁₉FN₅O₄

¹H NMR (400 MHz, DMSO-d₆) δ: 0.9 (d, 3H), 1.1 (d, 3H), 2.9 (d, 1H), 3.45 (d, 1H), 3.6 (m, 1H), 3.7 (m,1H), 3.8 (d,1H), 4.0 (d,1H), 6.9 (d, 1H), 7.5 (s, 1H), 7.7 (s, 1H), 11.5 (s, 1H), 11.85 (s, 1H), 12.45 (bs, 1H).

### Example 16

(2S,4R,4aR)-rel-9,10-Difluoro-8-(1H-imidazol-2-ylethynyl)-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-(1H-imidazol-2-ylethynyl)benzaldehyde (Intermediate 59).

MS (ES) M+H⁻: 456 for C₂₂H₁₉FN₅O₄

¹H NMR (400 MHz, DMSO-d₆) δ: 0.9 (d, 3H), 1.1 (d, 3H), 2.9 (d, 1H), 3.1 (m, 1H), 3.5 (d, 1H), 3.6 (m, 1H), 3.7 (m, 1H), 3.9 (d,1H), 4.1 (d, 1H), 7.1 (m, 2H), 7.2 (s, 1H).

### Example 17

### (2R,4S,4aS)-rel-9,10-Difluoro-2,4-dimethyl-8-{[5-(1H-pyrazol-5-yl)thiophen-2-yl]ethynyl}-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-{[5-(1H-pyrazol-5-yl)thiophen-2-yl]ethynyl}benzaldehyde (Intermediate 60).

MS (ES) M+H⁻: 436 for C₂₆H₂₁F₂N₅O₄S

¹H NMR (400 MHz, DMSO-d₆) δ: 0.9 (d, 3H), 1.1 (d, 3H), 2.9 (d, 1H), 3.1 (m, 1H), 3.45 (d, 1H), 3.6 (m, 1H), 3.7 (m, 1H), 3.9 (d,1H), 4.05 (d, 1H), 6.7 (s, 1H), 11.9 (s, 1H), 13.0 (s, 1H).

### Example 18

### (2R,4S,4aS)-rel-9,10-Difluoro-2,4-dimethyl-8-(pyridin-3-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-(pyridin-3-ylethynyl)benzaldehyde (Intermediate 61).

MS (ES) M+H⁻: 467 for C₂₄H₂F₂N₄O₄

¹H NMR (400 MHz, DMSO-d₆) δ: 0.9 (d, 3H), 1.1 (d, 3H), 2.9 (d, 1H), 3.1 (m, 1H), 3.5 (d, 1H), 3.6 (m, 1H), 3.7 (m,1H), 3.9 (d, 1H), 4.05 (d, 1H), 7.0 (d, 1H), 7.45 (m, 1H), 7.9 (d, 1H), 8.6 (d, 1H), 8.7 (s, 1H).

### Example 19

### (2S,4R,4aR)-rel-9,10-Difluoro-2,4-dimethyl-8-(pyrimidin-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-(pyrimidin-2-ylethynyl)benzaldehyde (Intermediate 62).

MS (ES) M+H⁻: 468 for C₂₃H₁₉F₂N₅O₄

¹H NMR (400 MHz, DMSO-d₆) δ: 0.9 (d, 3H), 1.1 (d, 3H), 2.9 (d, 1H), 3.1 (m, 1H), 3.5 (d, 1H), 3.65 (m, 1H), 3.8 (m, 1H), 3.9 (d, 1H), 4.1 (d, 1H), 7.1 (d, 1H), 7.5 (m, 1H), 8.8 (d, 2H), 11.6 (s, 1H), 11.9 (s, 1H).

### Example 20

### (2S,4R,4aR)-rel-9,10-Difluoro-2,4-dimethyl-8-(pyrazin-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3,4-difluoro-5-(pyrazin-2-ylethynyl)benzaldehyde (Intermediate 63).

MS (ES) M+H⁻: 468 for C₂₃H₁₉F₂N₅O₄

¹H NMR (400 MHz, DMSO-d₆) δ: 1.0 (d, 3H), 1.1 (d, 3H), 2.9 (d, 1H), 3.1 (m, 1H), 3.5 (d, 1H), 3.65 (m, 1H), 3.8 (m,1H), 3.9 (d, 1H), 4.1 (d, 1H), 7.1 (d, 1H), 8.6 (d, 1H), 8.7 (m, 1H), 8.8 (d, 1H).

### Example 21

### (2R,4S,4 aS)-rel-9,10-Difluoro-2,4-dimethyl-8-[(E)-(pyrrolidin-1-ylimino)methyl]-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-((2R,6S)-2,6-Dimethyl-morpholin-4-yl)-3,4-difluoro-5-[(Z)-pyrrolidin-1-ylimino methyl]-benzaldehyde (Intermediate 20).

MS(ESP): 461.4 (M+H) ¹H NMR (400 MHz, DMSO-d₆) δ: 0.8 (d, 3H), 1.1 (d, 3H),1.8 (s, 4H), 2.8 (d, 1H), 3.2 (t, 1H), 3.2 (s, 4H), 3.5 (t, 1H), 3.6 (t, 1H), 3.9 (d, 2H), 4.0 (d, 2H), 7.1 (d, 2H), 11.4 (s, 1H), 11.7 (s, 1H).

### Example 22

### (2R,4S,4aS)-rel-9,10-Difluoro-2,4-dimethyl-8-[(E)-(morpholin-4-ylimino)methyl]-1,2,4,4a-tetrahydro-2′H,6H-spiro[1,4-oxazino[4,3-a]quinoline-5,5′-pyrimidine]-2′,4′,6(1′H,3′H)-trione

Starting material: 2-[(2R,6S)-2,6-Dimethylmorpholin-4-yl]-3,4-difluoro-5-[(E)-(morpholin-4-ylimino)methyl]benzaldehyde (Intermediate 21).

MS(ESP): 477.4 (M+H)

¹H NMR (400 MHz, DMSO-d₆) δ: 0.8 (d, 3H), 1.1 (d, 3H), 2.8 (d, 1H), 3.0 (m, 5H), 3.5 (d, 1H), 3.6 (t, 1H), 3.7 (s, 5H), 3.8 (d, 1H), 4.0 (d, 1H), 7.1 (d, 1H), 7.5 (s, 1H), 11.4 (s, 1H), 11.7 (s, 1H).

### Example 23

### N′-{(E)-[(2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]methylidene}acetohydrazide

Starting material: N′-[(E)-{4-[(2R,6S)-2,6-Dimethylmorpholin-4-yl]-2,3-difluoro-5-formyl-phenyl}methylidene]acetohydrazide (Intermediate 22).

MS(ESP): 449 (M+H)

¹H NMR (400 MHz, DMSO-d₆) δ: 0.8 (d, 3H), 1.1 (d, 3H), 2.1 (s, 1H), 2.8 (d,1H), 3.0 (d, 1H), 3.6 (m, 2H), 3.7 (d, 1H), 3.8 (d, 1H), 4.0 (d, H), 7.2 (d,1H), 8.0 (s, 1H), 11.1 (s, 1H), 11.4 (s, 1H), 11.8 (s, 1H);

### Example 24

### (2R,4S,4aS)-rel-9-Fluoro-2,4-dimethyl-8-(1,3,4-thiadiazol-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-(1,3,4-thiadiazol-2-ylethynyl)benzaldehyde (Intermediate 64).

MS (ES) MH⁺: 456.2 for C₂₁H₁₈FN₅O₄S

¹H NMR (400 MHz, DMSO-d₆) δ: 0.9 (d, 3H), 1.1 (d, 3H), 2.8 (d, 1H), 2.9 (t, 1H), 3.4 (d, 1H), 3.5 (m, 1H), 3.6 (m, 1H), 3.8 (d, 1H), 4.15 (d, 1H), 7.0 (d, 1H), 7.2 (d, 1H), 9.7 (d, 1H), 11.5 (s, 1H), 11.8 (s, 1H).

### Example 25

### (2R,4S,4aS)-rel-9-Fluoro-2,4-dimethyl-8-[(5-methyl-1,3,4-thiadiazol-2-yl)ethynyl]-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-[(5-methyl-1,3,4-thiadiazol-2-yl)ethynyl]benzaldehyde (Intermediate 65).

MS (ES) MH⁺: 470.2 for C₂₂H₂₀FN₅O₄S

¹H NMR (400 MHz, DMSO-d₆) δ: 0.9 (d, 3H), 1.1 (d, 3H), 2.75 (s, 3H), 2.8 (d, 1H), 2.9 (t, 1H), 3.4 (d, 1H), 3.5 (m, 1H), 3.6 (m, 1H), 3.8 (d, 1H), 4.1 (d, 1H), 6.95 (d, 1H), 7.2 (d, 1H), 11.5 (bs, 1H), 11.8 (bs, 1H).

### Example 26

### (2R,4S,4aS)-rel-9-Fluoro-2,4-dimethyl-8-(1,3-thiazol-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-(1,3-thiazol-2-ylethynyl)benzaldehyde (Intermediate 66).

MS (ESP) MH⁺: 455.2 for C₂₂H₁₉FN₄O₄S

¹H NMR (400 MHz, DMSO-d₆) δ: 0.9 (d, 3H), 1.1 (d, 3H), 2.75 (s, 3H), 2.8 (d, 1H), 2.9 (t, 1H), 3.4 (d, 1H), 3.5 (m, 1H), 3.6 (m, 1H), 3.8 (d, 1H), 4.1 (d, 1H), 6. (d, 1H), 7.1 (d, 1H), 8.1 (d, 1H), 9.1 (d, 1H), 11.5 (bs, 1H), 11.8 (bs, 1H).

### Example 27

### (2R,4S,4 aS)-rel-9-Fluoro-2,4-dimethyl-8-(1,3-thiazol-5-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-(1,3-thiazol-5-ylethynyl)benzaldehyde (Intermediate 67).

MS (ES) MH⁺: 479.2 for C₂₂H₁₉FN₄O₄S

¹H NMR (400 MHz, DMSO-d₆) δ: 0.9 (d, 3H), 1.1 (d, 3H), 2.9 (d, 1H), 3.0 (t, 1H), 3.4 (d, 1H), 3.60 (d, 1H), 3.62 (m, 1H), 3.7 (t, 1H), 3.8 (d, 1H), 4.0 (d, 1H), 7.0 (s, 1H), 7.2 (d, 1H), 8.1 (s, 1H), 9.1(s, 1H), 11.5 (s, 1H), 11.8 (s, 1H).

### Example 28

### (2R,4S,4aS)-rel-9-Fluoro-2,4-dimethyl-8-(thiophen-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethyl morpholin-4-yl]-4-fluoro-5-(thiophen-2-ylethynyl)benzaldehyde (Intermediate 68).

MS (ES) MH⁺: 452.2 for C₂₃H₂₀FN₃O₄S

¹H NMR (400 MHz, DMSO-d₆) δ: 0.9 (d, 3H), 1.1 (d, 3H), 2.8-2.9 (m, 2H), 3.4 (s, 1H), 3.5-3.6 (m, 2H), 3.8 (d, 1H), 4.1 (d, 1H), 6.9 (d, 1H), 7.1 (m, 2H), 7.3 (m, 1H), 7.6 (dd, 1H), 11.5 (s, 1H), 11.8 (s, 1H).

### Example 29

### (2R,4S,4aS)-rel-8-(1-Benzothiophen-2-ylethynyl)-9-fluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 5-(1-Benzothiophen-2-ylethynyl)-2-[(2R,6S)-2,6-dimethylmorpholin-4-yl]-4-fluorobenzaldehyde (Intermediate 69).

MS (ES) MH⁺: 504.2 for C₂₇H₂₂FN₃O₄S

¹H NMR (400 MHz, DMSO-d₆) δ: 0.9 (d, 3H), 1.1 (d, 3H), 2.75 (s, 3H), 2.8 (d, 1H), 2.9 (t, 1H), 3.4 (d, 1H), 3.5 (m, 1H), 3.6 (m, 1H), 3.8 (d, 1H), 4.12 (d, 1H), 6.9 (d, 1H), 7.1 (d, 1H), 7.4 (m, 2H), 7.65 (s, 1H), 7.8 (m, 1H), 7.9 (m, 1H), 11.5 (bs, 1H), 11.8 (bs, 1H).

### Example 30

### (2R,4S,4aS)-rel-9-Fluoro-2,4-dimethyl-8-[(1-methyl-1H-imidazol-2-yl)ethynyl]-1,2,4,4a-tetrahydro-2′H,6H-spiro[1,4-oxazino[4,3-a]quinoline-5,5′-pyrimidine]-2′,4′,6(1′H,3′H)-trione

Starting material: 2-[(2R,6S)-2,6-Dimethylmorpholin-4-yl]-4-fluoro-5-[(1-methyl-1H-imidazol-2-yl)ethynyl]benzaldehyde (Intermediate 70).

MS (ES) MH⁺: 452.2 for C₂₃H₂₂FN₅O₄

¹H NMR (400 MHz, DMSO-d₆) δ: 0.9 (d, 3H), 1.1 (d, 3H), 2.8 (d, 1H), 2.9 (m, 1H), 3.4 (d, 1H), 3.5 (m, 1H), 3.7 (s, 3H), 3.8 (d, 1H), 3.8 (d, 1H); 4.1 (d, 1H), 6.9 (t, 3H), 7.1 (s, 1H), 7.3 (s, 2H) 11.5 (s, 1H), 11.8 (s, 1H).

### Example 31

### (2R,4S,4aS)-rel-9-Fluoro-2,4-dimethyl-8-(pyrazin-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-(pyrazin-2-ylethynyl)benzaldehyde (Intermediate 72).

MS (ES) MH⁺: 450.2 for C₂₃H₂₀FN₅O₄

¹H NMR (400 MHz, DMSO-d₆) δ: 0.9 (d, 3H), 1.1 (d, 3H), 2.9 (d, 1H), 3.0 (t, 1H), 3.4 (d, 1H), 3.6 (m, 1H), 3.7 (s, 3H), 3.8 (d, 1H); 4.0 (d, 1H), 7.1 (s, 1H), 7.3 (d, 1H), 8.6 (d, 2H), 8.6 (d 1H), 8.8 (d, 1H), 11.5 (s, 1H), 11.8 (s, 1H).

### Example 32

### (2R,4S,4aS)-rel-9-Fluoro-2,4-dimethyl-8-(pyridin-3-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-(pyridin-3-ylethynyl)benzaldehyde (Intermediate 73).

MS (ES) MH⁺: 447.2 for C₂₄H₂₁FN₄O₄

¹H NMR (400 MHz, DMSO-d₆) δ: 0.9 (d, 3H), 1.1 (d, 3H), 2.8 (d, 1H), 2.9 (m, 1H), 3.35 (d, 1H), 3.5-3.6 (m, 2H), 3.8 (d, 1H), 3.8 (d, 1H), 4.1 (d, 1H), 6.9 (d, 1H), 7.1 (d, 1H), 7.4 (dd, 1H), 7.9 (m, 1H), 8.5 (dd, 1H), 8.7 (d, 1H), 11.5 (bs, 1H), 11.8 (bs, 1H).

### Example 33

### (2R,4S,4aS)-rel-9-Fluoro-2,4-dimethyl-8-(pyrimidin-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-(pyrimidin-2-ylethynyl)benzaldehyde (Intermediate 74).

MS (ES) MH⁺: 450.2 for C₂₃H₂₀FN₅O₄

¹H NMR (400 MHz, DMSO-d₆) δ: 0.91 (d, 3H), 1.12 (d, 3H), 2.80 (d, 1H), 2.88 (m, 1H), 3.35 (d, 1H), 3.49-3.60 (m, 2H), 3.79 (d, 1H), 3.84 (d, 1H), 4.13 (d, 1H), 6.91 (d, 1H), 7.14 (d, 1H), 7.43 (t, 1H), 8.77 (d, 1H), 11.53 (bs, 1H), 11.83 (bs, 1H).

### Example 34

### (2R,4S,4aS)-rel-8-(1,3-Benzothiazol-2-ylethynyl)-9-fluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 5-(1,3-Benzothiazol-2-ylethynyl)-2-[(2R,6S)-2,6-dimethylmorpholin-4-yl]-4-fluorobenzaldehyde (Intermediate 75).

MS (ES) MH⁺: 492.2 for C₂₆H₂₁FN₄O₄S

¹H NMR (400 MHz, DMSO-d₆) δ: 0.9 (d, 3H), 1.1 (d, 3H), 2.8 (d, 1H), 2.9 (m, 1H), 3.35 (d, 1H), 3.5-3.6 (m, 2H), 3.8 (d, 1H), 3.8 (d, 1H), 4.2 (d, 1H), 7.0 (d, 1H), 7.2 (d, 1H), 7.5 (t, 1H), 7.6 (m, 1H), 8.0 (d, 1H), 8.1 (d, 1H), 11.6 (bs, 1H), 11.9 (bs, 1H).

### Example 35

### (2R,4S,4aS)-rel-8-(1,3-Benzoxazol-2-ylethynyl)-9-fluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 5-(1,3-Benzoxazol-2-ylethynyl)-2-[(2R,6S)-2,6-dimethylmorpholin-4-yl]-4-fluorobenzaldehyde (Intermediate 83).

MS (ES) MH⁺: 489.2 for C₂₆H₂₁FN₄O₅

¹H NMR (400 MHz, DMSO-d₆) δ: 0.9 (d, 3H), 1.1 (d, 3H), 2.8 (d, 1H), 2.9 (m, 1H), 3.35 (d, 1H), 3.5-3.6 (m, 2H), 3.8 (d, 1H), 4.2 (d, 1H), 7.0 (d, 1H), 7.2 (d, 1H), 7.4-7.5 (m, 2H), 7.7-7.8 (m, 2H), 11.6 (bs, 1H), 11.8 (bs, 1H).

### Example 36

### (2R,4S,4aS)-rel-10-Fluoro-2,4-dimethyl-8-(1,3,4-thiadiazol-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-4-fluoro-5-(1,3,4-thiadiazol-2-ylethynyl)benzaldehyde (Intermediate 76).

MS (ES) MH⁺: 456.2 for C₂₁H₁₈FN₅O₄S

¹H NMR (400 MHz, DMSO-d₆) δ: 0.88 (d, 3H), 1.10 (d, 3H), 2.90 (d, 1H), 3.02 (t, 1H), 3.37 (m, 1H), 3.63 (m, 1H), 3.71 (m, 1H), 3.88 (d, 1H), 4.06 (d, 1H), 7.10 (s, 1H), 7.36 (d, 1H), 9.67(d, 1H), 11.5 (bs, 2H).

### Example 37

### (2R,4S,4aS)-rel-10-Fluoro-2,4-dimethyl-8-[(5-methyl-1,3,4-thiadiazol-2-yl)ethynyl]-1,2,4,4a-tetrahydro-2′H,6H-spiro[1,4-oxazino[4,3-a]quinoline-5,5′-pyrimidine]-2′,4′,6(1′H,3′H)-trione

Starting material: 2-[(2R,65)-2,6-Dimethylmorpholin-4-yl]-3-fluoro-5-[(5-methyl-1,3,4-thiadiazol-2-yl)ethynyl]benzaldehyde (Intermediate 77).

MS (ES) MH⁺: 470.2 for C₂₂H₂₀FN₅O₄S

¹H NMR (400 MHz, DMSO-d₆) δ: 0.9 (d, 3H), 1.1 (d, 3H), 2.75 (s, 3H), 2.9 (d, 1H), 3.0 (t, 1H), 3.45 (d, 1H), 3.6 (q, 1H), 3.7 (m, 1H), 3.9 (d, 1H), 4.1 (d, 1H), 7.1 (s, 1H), 7.35 (d, 1H), 11.5 (s, 1H), 11.9 (s, 1H).

### Example 38

### (2R,4S,4aS)-rel-10-Fluoro-2,4-dimethyl-8-(1,3-thiazol-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,65)-2,6-Dimethylmorpholin-4-yl]-3-fluoro-5-(1,3-thiazol-2-ylethynyl)benzaldehyde (Intermediate 78).

MS (ESP) MH⁺: 455.2 for C₂₂H₁₉FN₄O₄S

¹H NMR (400 MHz, DMSO-d₆) δ: 0.9 (d, 3H), 1.1 (d, 3H), 2.9 (d, 1H), 2.99 (t, 1H), 2.9 (d, 1H), 3.5 (d, 1H), 3.6 (m, 1H), 3.74 (m, 1H), 3.86 (d, 1H), 4.1 (d, 1H), 4.1 (m, 2H), 7.0 (s, 1H), 7.3 (d, 1H), 7.85 (s, 1H), 7.95 (s, 1H), 11.5 (bs, 1H), 11.85 (bs, 1H).

### Example 39

### (2R,4S,4aS)-rel-10-Fluoro-2,4-dimethyl-8-(1,3-thiazol-5-ylethynyl)-1,2,4,4a-tetrahydro-2′H,6′-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3-fluoro-5-(1,3-thiazol-5-ylethynyl)benzaldehyde (Intermediate 79).

MS (ES) MH⁺: 479.2 for C₂₂H₁₉FN₄O₄S

¹H NMR (400 MHz, DMSO-d₆) δ: 0.9 (d, 3H), 1.1 (d, 3H), 2.9 (d, 1H), 3.0 (t, 1H), 3.4 (d, 1H), 3.6 (d, 1H), 3.6 (m, 1H), 3.7 (t, 1H), 3.8 (d, 1H), 4.0 (d, 1H), 7.0 (s, 1H), 7.2 (d, 1H), 8.1 (s, 1H), 9.1 (s, 1H), 11.5 (s, 1H), 11.8 (s, 1H).

### Example 40

### (2R,4S,4aS)-rel-10-Fluoro-2,4-dimethyl-8-(thiophen-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3-fluoro-5-(thiophen-2-ylethynyl)benzaldehyde (Intermediate 80).

MS (ES) MH⁺: 454.2 for C₂₃H₂₀FN₃O₄S

¹H NMR (400 MHz, DMSO-d₆) δ: 0.9 (d, 3H), 1.1 (d, 3H), 2.9 (d, 1H), 3.0 (t, 1H), 3.35 (d, 1H), 3.55 (m, 1H), 3.7 (m, 1H), 3.9 (d, 1H), 4.0 (d, 1H), 6.95 (s, 1H), 7.1 (d, 1H), 7.3 (d, 1H), 7.6 (d, 1H), 11.5 (bs, 1H), 11.8 (bs, 2H).

### Example 41

### (2R,4S,4aS)-rel-8-(1-Benzothiophen-2-ylethynyl)-10-fluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 5-(1-Benzothiophen-2-ylethynyl)-2-[(2R,6S)-2,6-dimethylmorpholin-4-yl]-3-fluorobenzaldehyde (Intermediate 81).

MS (ES) MH⁺: 504.2 for C₂₇H₂₂FN₃O₄S

¹H NMR (400 MHz, DMSO-d₆) δ: 0.89 (d, 3H),1.11 (d, 3H), 2.92 (d, 1H), 3.01 (m, 2H), 3.34 (d, 1H), 3.64 (m, 1H), 3.74 (m, 1H), 3.86 (d, 1H), 4.06 (d, 1H), 7.03 (s, 1H), 7.25 (d, 1H), 7.42 (dd, 1H), 7.66 (s, 1H), 7.85 (dd, 1H), 7.96 (dd, 1H), 11.48 (bs, 1H), 11.83 (bs, 1H).

### Example 42

### (2R,4S,4aS)-rel-10-Fluoro-2,4-dimethyl-8-[(1-methyl-1H-imidazol-2-yl)ethynyl]-1,2,4,4a-tetrahydro-2′H,6H-spiro[1,4-oxazino[4,3-a]quinoline-5,5′-pyrimidine]-2′,4′,6(1′H,3′H)-trione

Starting material: 2-[(2R,6S)-2,6-Dimethylmorpholin-4-yl]-3-fluoro-5-[(1-methyl-1H-imidazol-2-yl)ethynyl]benzaldehyde (Intermediate 82).

MS (ES) MH⁺: 452.2 for C₂₃H₂₂FN₅O₄

¹H NMR (400 MHz, DMSO-d₆) δ: 0.88 (d, 3H), 1.10 (d, 3H), 2.91 (d, 1H), 2.99 (t, 1H), 3.44 (d, 1H), 3.62 (m, 1H), 3.73 (s, 3H); 3.84 (d, 1H); 4.04 (d, 1H), 6.95 (s, 1H), 7.02 (s, 1H), 7.23 (d, 2H) 11.49 (s, 1H), 11.83 (s, 1H).

### Example 43

### (2R,4S,4aS)-rel-10-Fluoro-2,4-dimethyl-8-(pyrazin-2-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3-fluoro-5-(pyrazin-2-ylethynyl)benzaldehyde (Intermediate 84).

MS (ES) MH⁺: 450.2 for C₂₃H₂₀FN₅O₄

¹H NMR (400 MHz, DMSO-d₆) δ: 0.9 (d, 3H), 1.1 (d, 3H), 2.9 (d, 1H), 3.0 (t, 1H), 3.4 (d, 1H), 3.6 (m, 1H), 3.7 (s, 3H), 3.8 (d, 1H); 4.0 (d, 1H), 7.1 (s, 1H), 7.3 (d, 1H), 8.57 (d, 2H), 8.63 (d, 1H), 8.8 (d, 1H), 11.5 (s, 1H), 11.8 (s, 1H).

### Example 44

### (2R,4S,4aS)-rel-10-Fluoro-2,4-dimethyl-8-(pyridin-3-ylethynyl)-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3-fluoro-5-(pyridin-3-ylethynyl)benzaldehyde (Intermediate 85).

MS (ES) MH⁺: 447.2 for C₂₄H₂₁FN₄O₄

¹H NMR (400 MHz, DMSO-d₆) δ: 0.9 (d, 3H), 1.109 (d, 3H), 2.9 (d, 1H), 3.0 (t, 1H), 3.4 (d, 1H), 3.6 (m, 1H), 3.7 (s, 3H), 3.8 (d, 1H); 4.0 (d, 1H), 7.0 (s, 1H), 7.2 (d, 1H), 7.4 (dd, 1H), 7.4 (dd, 1H), 7.9 (dd, 1H), 8.5 (d, 2H), 8.7 (d, 1H), 11.5 (s, 1H), 11.8 (s, 1H).

### Example 45

### (2R,4S,4aS)-rel-10-Fluoro-2,4-dimethyl-8-(pyrimidin-2-ylethynyl)-1,2,4,4a-tetrahydro-2′H,6′H-spiro[1,4-oxazino[4,3-a]quinoline-5,5′-pyrimidine]-2′,4′,6′(1′H,3′H)-trione

Starting material: 2-[(2R,6S)-2,6-Dimethylmorpholin-4-yl]-3-fluoro-5-(pyrimidin-2-ylethynyl)benzaldehyde (Intermediate 86).

MS (ES) MH⁺: 450.2 for C₂₃H₂₀FN₅O₄

¹H NMR (400 MHz, DMSO-d₆) δ: 0.9 (d, 3H), 1.1 (d, 3H), 2.9 (d, 1H), 3.0 (t, 1H), 3.4 (d, 1H), 3.6 (m, 1H), 3.7 (s, 3H), 3.8 (d, 1H); 4.0 (d, 1H), 7.1 (s, 1H), 7.3 (d, 1H), 7.45 (dd, 1H), 7.9 (dd, 1H), 8.8 (m, 2H), 11.5 (bs, 1H), 11.8 (bs, 1H).

### Example 46

### (2R,4S,4aS)-rel-8-(1,3-Benzothiazol-2-ylethynyl)-10-fluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 5-(1,3-Benzothiazol-2-ylethynyl)-2-[(2R,6S)-2,6-dimethylmorpholin-4-yl]-3-fluorobenzaldehyde (Intermediate 71).

MS (ES) MH⁺: 492.2 for C₂₆H₂₁FN₄O₄S

¹H NMR (400 MHz, DMSO-d₆) δ: 0.9 (d, 3H), 1.1 (d, 3H), 2.9 (d, 1H), 3.0 (t, 1H), 3.4 (d, 1H), 3.6 (m, 1H), 3.7 (s, 3H), 3.8 (d, 1H); 4.1 (d, 1H), 7.1 (s, 1H), 7.4 (d, 1H), 7.6 (m, 2H), 8.0 (d, 1H), 8.1 (d, 2H), 11.5 (s, 1H), 11.9 (s, 1H).

### Example 47

### (2R,4S,4aS)-rel-8-{(E)-[(3,5-Dimethyl-4H-1,2,4-triazol-4-yl)imino]methyl}-9,10-difluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

To a solution of (2R,4S,4aS)-9,10-difluoro-2,4-dimethyl-2′,4′,6′-trioxo-1,1-1,1′,2,3′,4,4′,4a,6′-octahydro-2′H,6H-spiro[1,4-oxazino[4,3-a]quinoline-5,5′-pyrimidine]-8-carbaldehyde (Intermediate 25, 250 mg, 0.63 mmol) in EtOH, was added 4-amino 3,5-dimethyl triazole (71 mg, 0.636 mmol) followed by glacial acetic acid (2 drops). The reaction was mixture heated to 85° C. for 12 hours, cooled to room temperature, and concentrated under reduced pressure. The residue thus obtained was purified using normal phase HPLC (95:5: Hexane:IPA) to give the title compound as a pale yellow solid. Yield: 50 mg (20%).

MS(ES)MH⁺: 487 for C₂₂H₂₃F₂N₇O₄

¹H NMR (400 MHz, DMSO-d₆) δ: 0.9 (d, 3H), 1.1 (d, 3H), 2.3 (s, 6H), 2.9 (d, 1H), 3.1 (t, 1H), 3.6 (d, 2H), 3.7 (s, 1H), 3.9 (d, 1H), 4.1 (d, 1H), 7.4 (d, 1H), 8.7 (s, 1H), 11.4 (s, 1H), 11.8 (s, 1H).

### Example 48

### N′-{(1E)-1-[(2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]ethylidene}acetohydrazide

To a solution of (2R,4S,4aS)-8-acetyl-9,10-difluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione (Intermediate 29, 250 mg, 0.63 mmol) in EtOH, was added acetic hydrazide (47 mg, 0.636 mmol) followed by glacial acetic acid (2 drops). The mixture was heated to 85° C. for 12 hours, cooled to room temperature, and concentrated under reduced pressure. The residue thus obtained was purified using normal phase HPLC (95:5: Hexane:IPA) to give the title compound as a pale yellow solid. Yield: 50 mg (20%).

MS(ES)MH⁺: 464.2 for C₁₉H₁₉F₂N₃O₅

¹H NMR (400 MHz, DMSO-d₆) δ: 0.8 (d, 3H), 0.9 (d, 3H), 1.9 (d, 1H), 2.1 (d, 5H), 2.8 (t, 1H), 3.3 (m, 1H), 3.4 (d, 1H), 3.6 (d, 1H), 3.7 (s, 1H), 3.8 (d, 1H), 4.0 (d, 1H), 7.5 (d, 1H), 10.3 (d,1H), 11.5 (s, 1H), 11.8 (s, 1H).

Examples 49 to 60 were synthesized from (2R,4S,4aS)-8-acetyl-9,10-difluoro-2,4-dimethyl-1,2,4,4a-tetrahydro-2′H,6H-spiro[1,4-oxazino[4,3-c]quinoline-5,5′-pyrimidine]-2′,4′,6′(1′H,3′H)-trione (Intermediate 29) and the indicated starting materials, using a method similar to the one described for the synthesis of Example 48.

### Example 49

### Methyl (2E)-2-{1-[(2R,4S,4aS)-rel-9,10-difluoro-2,4-dimethyl-2′,4′6′-trioxo-1,1′,2,3′,4,4′,4a,6′-octahydro-2′H,6H-spiro[1,4-oxazino[4,3-a]quinoline-5,5′-pyrimidin]-8-yl]ethylidene}hydrazinecarboxylate

Starting material: Methoxycarbonylhydrazine.

MS (ES) MH⁺: 480.0 for C₂₁H₂₃F₂N₅O₆

¹H NMR (400 MHz, DMSO-d₆) δ: 1.09 (d, 3H), 1.1 (d, 3H), 2.1 (s, 3H), 2.4 (s, 1H), 2.49 (d, 1H), 3.0 (t, 1H), 3.5 (d, 1H), 3.6 (d, 1H), 3.7 (s, 3H), 3.79 (d, 1H), 3.8 (d, 1H), 3.9 (d, 1H), 7.0 (d, 1H), 10.0 (s, 1H), 11.47(s, 1H), 11.8 (s,1H).

### Example 50

### tert-Butyl (2E)-2-{1-[(2R,4S,4aS)-rel-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]ethylidene}hydrazinecarboxylate

Starting material: t-Butylcarbazate.

MS (ES) MH⁻: 520.0 for C₂₄H₂₉F₂N₅O₅

¹H NMR (400 MHz, DMSO-d₆) δ: 1.05 (d, 3H), 1.09 (d, 3H), 1.5 (s, 1H), 2.1 (s, 3H), 2.8 (d, 1H), 2.9 (t, 1H), 3.3 (d, 1H), 3.6 (t, 1H), 3.8(d, 1H), 3.9 (d, 1H), 4.0 (d, 1H), 7.0 (d, 1H), 9.7 (s, 1H), 11.5 (s, 1H), 11.8 (s,1H).

### Example 51

### (2E)-2-{1-[(2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]ethylidene}-N-phenylhydrazinecarboxamide

Starting material: 4-Phenylsemicarbazide.

MS (ES) MH⁺: 541.2 for C₂₆H₂₆F₂N₆O₅

¹H NMR (400 MHz, DMSO-d₆) δ: 0.89 (d, 3H), 1.1 (d, 3H), 2.0 (s, 3H), 2.9 (d, 1H), 3.0 (d, 1H), 3.1 (d, 1H), 3.4 (d, 1H), 3.6 (t, 1H), 3.7 (d, 1H), 3.9 (d, 1H), 6.9 (d, 1H), 7.0 (d, 1H), 7.3 (t, 2H), 7.5 (d, 2H), 8.69 (s, 1H), 9.8 (s, 1H), 11.5 (s, 1H), 11.8 (s,1H).

### Example 52

### (2R,4S,4aS)-rel-8-[(1E)-N-(2,4-Dioxoimidazolidin-1-yl)ethanimidoyl]-9,10-difluoro-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione

Starting material: 1-Amino-2,4-imidazolidinedione.

MS (ES) MH⁺: 505.2 for C₂₂H₂₂F₂N₆O₆

¹H NMR (400 MHz, DMSO-d₆) δ: 0.8 (d, 3H), 1.0 (d, 3H), 2.2 (s, 3H), 2.5 (d, 1H), 2.8 (t, 1H), 3.0 (d, 1H), 3.3 (d, 1H), 3.5 (d, 1H), 3.7 (d, 1H), 3.8 (d, 1H), 4.2 (s, 2H), 7.1 (d, 1H), 11.2 (s, 1H), 11.5 (s, 1H), 11.8 (s,1H).

### Example 53

### 2-Cyano-N′-{(1E)-1-[(2R,4S,4aS)-rel-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]ethylidene}acetohydrazide

Starting material: 2-Cyanoacethydrazide.

MS (ES) MH⁺: 489.2 for C₂₂H₂₂F₂N₆O₅

¹H NMR (400 MHz, DMSO-d₆)) δ: 0.9 (d, 3H), 1.2 (d, 3H), 2.2 (s, 3H), 2.8 (d, 1), 2.9 (t, 1H), 3.0 (d, 1H), 3.3 (d, 1H), 3.6 (t, 1H), 3.7 (m, 1H), 3.8 (d, 1H), 4.1 (d, 2H), 7.1 (s, 1H), 11.3 (s, 2H).

### Example 54

### (2E)-2-{1-[(2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]ethylidene}hydrazinecarboxamide

Starting material: Semicarbazide.

MS (ES) MH⁻: 465.0 for C₂₀H₂₂F₂N₆O₅

¹H NMR (400 MHz, DMSO-d₆)) δ: 0.8 (d, 3H), 1.0 (d, 3H), 2.1 (s, 3H), 2.4 (d, 1H), 2.8 (d, 1H), 3.0 (t, 1H), 3.4 (d, 2H), 3.6 (t, 1H), 3.7 (m, 1H), 3.9 (d, 1H), 4.0 (d, 2H), 6.4 (s, 2H), 7.2 (d, 1H), 9.3 (d, 1H), 11.4 (s, 1H), 11.8 (s,1H).

### Example 55

### N′-{(1E)-1-[(2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]ethylidene}-2-methoxyacetohydrazide

Starting material: Methoxyacetohydrazide.

MS (ES) MH⁺: 494.2 for C₂₂H₂₅F₂N₅O₆

¹H NMR (400 MHz, DMSO-d₆) δ: 0.8 (d, 3H), 1.0 (d, 3H), 2.8 (d, 1H), 2.9 (t, 1H), 3.4 (d, 3H), 3.5 (d, 1H), 3.6 (d, 1H), 3.7 (d, 1H), 3.8 (d, 1H), 4.0 (d, 2H), 4.3 (s, 1H), 7.0 (d, 1H), 10.3 (d, 1H), 11.49(s, 1H), 11.8 (s,1H).

### Example 56

### N′-{(1E)-1-[(2R,4S,4aS)-rel-9,10-Difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]ethylidene}cyclopropanecarbohydrazide

Starting material: Cyclopropanecarbohydrazide.

MS (ES) MH⁻: 488.2 for C₂₃H₂₅F₂N₅O₅

¹H NMR (400 MHz, DMSO-d₆) δ: 0.7 (d, 3H), 0.89 (d, 3H), 1.1 (d, 3H), 1.9 (s, 1H), 2.1 (d, 3H), 2.4 (s, 1H), 2.6 (t, 1H), 2.8 (t, 1H), 3.0 (d, 1H), 3.4 (d, 1H), 3.7 (d, 1H), 3.8 (d, 2H), 4.0 (d, 1H), 6.9 (d, 1H), 10.5 (d, 1H), 11.4 (s, 1H), 11.8 (s,1H).

### Example 57

### (2R,4S,4aS)-rel-9,10-Difluoro-2,4-dimethyl-8-{(1E)-1-[2-(pyridin-2-yl)hydrazinylidene]ethyl}-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-Pyridylhydrazine.

MS (ES) MH⁺: 499.2 for C₂₄H₂₄F₂N₆O₄

¹H NMR (400 MHz, DMSO-d₆) δ: 0.9 (d, 3H), 1.1 (d, 3H), 2.2 (s, 3H), 2.8 (d, 1H), 2.9 (t, 1H), 3.5 (d, 1H), 3.8 (t, 1H), 3.85(d, 2H), 4.0 (d, 1H), 6.7 (m, 1H), 7.0 (d, 1H), 7.6 (m, 1H), 8.1 (d, 1H), 9.6 (s, 1H), 11.5 (s, 1H), 11.8 (s,1H).

### Example 58

### (2R,4S,4aS)-rel-9,10-Difluoro-2,4-dimethyl-8-[(1E)-1-(2-phenylhydrazinylidene)ethyl]-1,2,4,4a-tetrahydro-2′H,6H-spiro[1,4-oxazino[4,3-a]quinoline-5,5′-pyrimidine]-2′,4′,6′(1′H,3′H)-trione

Starting material: Phenylhydrazine.

MS (ES) MH⁺: 498.2 for C₂₅H₂₅F₂N₅O₄

¹H NMR (400 MHz, DMSO-d₆) δ: 0.8 (d, 3H), 1.01 (d, 3H), 2.18 (s, 3H), 2.8 (d, 1H), 3.0 (t, 1H), 3.5 (d, 1H), 3.6 (m, 1H), 3.79(s, 1H), 3.9 (d, 2H), 4.03(d, 1H), 6.7 (d, 1H), 7.0 (d, 1H), 7.1 (m, 4H), 9.1 (s, 1H), 11.47 (s, 1H), 11.8 (s,1H).

### Example 59

### (2R,4S,4aS)-rel-9,10-Difluoro-2,4-dimethyl-8-{(1E)-1-[2-(pyrimidin-2-yl)hydrazinylidene]ethyl}-1,2,4,4a-tetrahydro-2′H,6H-spiro[1,4-oxazino[4,3-a]quinoline-5,5′-pyrimidine]-2′,4′,6′(1′H,3′H)-trione

Starting material: 2-Pyrimidinylhydrazine.

MS (ESP) MH⁺: 500.2 for C₂₃H₂₃F₂N₇O₄

¹H NMR (400 MHz, DMSO-d₆) δ: 0.89 (d, 3H), 1.01 (d, 3H), 2.18 (s, 3H), 2.8 (d, 1H), 3.0 (t, 1H), 3.3 (d, 1H), 3.5 (s, 1H), 3.66(d, 1H), 3.8 (d, 1H), 4.05(d, 1H), 6.8 (d, 1H), 7.0 (d, 1H), 8.4 (s, 2H), 10.06 (s, 1H), 11.5 (s, 1H), 11.8 (s,1H).

### Example 60

### (2R,4S,4aS)-rel-9,10-Difluoro-2,4-dimethyl-8-{(1E)-1-[2-(pyrazin-2-yl)hydrazinylidene]ethyl}-1,2,4,4a-tetrahydro-2′H,6H-spiro[1,4-oxazino[4,3-a]quinoline-5,5′-pyrimidine]-2′,4′,6′(1′H,3′H)-trione

Starting material: Pyrazinylhydrazine.

MS (ESP) MH⁺: 500.2 for C₂₃H₂₃F₂N₇O₄

¹H NMR (400 MHz, DMSO-d₆) δ: 0.9 (d, 3H), 1.1 (d, 3H), 2.1 (s, 3H), 2.86 (d, 1H), 3.0 (t, 1H), 3.6 (m, 1H), 3.7 (d, 1H), 3.8(d, 1H), 4.05 (d, 1H), 7.1(d, 1H), 7.8 (s, 1H), 8.0 (s, 1H), 8.6 (s, 2H), 10.01 (s, 1H), 11.5 (s, 1H), 11.8 (s,1H).

### Example 61

### (2R,4S,4aS)-rel-9,10-Difluoro-2,4-dimethyl-2′,4′,6′-trioxo-N-(2-oxotetrahydrofuran-3-yl)-1,1′,2,3′,4,4′,4a,6′-octahydro-2′H,6H-spiro[1,4-oxazino[4,3-a]quinoline-5,5′-pyrimidine]-8-carboxamide

To a solution of (2R,4S,4aS)-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidine]-8-carboxylic acid (Intermediate 28, 1.0 eq) and TBTU (2.5 eq) and 3-amino-dihydro-furan-2-one (1.1 eq) in anhydrous MDC, at 0° C. was added DIPEA (2 eq). The mixture was warmed to room temperature and stirred 12 hours. The reaction mixture was then diluted with MDC and washed successively with water and brine. The organic layer was dried over anhydrous sodium sulfate, filtered, and concentrated. The residue thus obtained was purified over silica gel flash column using a gradient of ethyl acetate in petroleum ether to give the title product as a solid.

MS (ES) MH⁺: 493.2 for C₂₂H₂₂F₂N₄O₇

¹H NMR (400 MHz, CD₃OD) δ: 1.0 (d, 3H),1.3 (d, 3H), 2.4 (q, 1H), 2.6 (p, 1H), 3.1 (t, 2H), 3.3 (s, 1H), 3.8 (p, 1H), 3.9 (m, 1H), 4.0 (d, 1H), 4.2 (d, 1H), 4.3 (q, 1H), 4.5 (q, 1H), 4.7 (q, 1H), 7.2(d, 1H).

Examples 62 to 69 were synthesized from (2R,4S,4aS)-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidine]-8-carboxylic acid (Intermediate 28) and the indicated starting materials using a method similar to the one described for the synthesis of Example 61.

### Example 62

### (2R,4S,4aS)-rel-N-(1, 1-Dioxidotetrahydrothiophen-3-yl)-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidine]-8-carboxamide

Starting material: 1,1-Dioxotetrahydrothiophen-3-yl)amine.

MS (ES) MH⁺: 527.2 for C₂₂H₂₄F₂N₄O₇S

¹H NMR (400 MHz, DMSO-d₆) δ: 0.9 (d, 3H), 1.1 (d, 3H), 2.2 (m, 1H), 2.4 (m, 1H), 2.8 (d, 1H), 3.1 (m, 2H), 3.2 (q, 1H), 3.5 (m, 2H), 3.7 (m, 1H), 3.8 (m, 1H), 3.9 (d, 1H), 4.1 (d, 1H), 4.6 (q, 1H), 7.1 (d, 1H), 8.3 (d, 1H), 11.7 (bs, 2H).

### Example 63

### tert-Butyl 3-({[2R,4S,4aS)-rel-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]carbonyl}amino)pyrrolidine-1-carboxylate

Starting material: 3-Amino-1-(tert-butoxycarbonyl)pyrrolidine.

MS (ES) MH⁺: 527.2 for C₂₇H₃₃F₂N₅O₇

¹H NMR (400 MHz, DMSO-d₆) δ: 0.9 (d, 3H), 1.1 (d, 3H), 1.4 (d, 9H), 1.8 (bs, 1H), 2.1 (bs, 1H), 2.8 (d, 1H), 3.1 (t, 1H), 3.2 (m, 1H), 3.3 (m, 1H), 3.5 (m, 2H), 3.6 (m, 1H), 3.8 (m, 1H), 3.9 (d, 1H), 4.1 (d, 1H), 4.3 (m, 1H), 7.0 (d, 1H), 8.2 (bs, 1H), 11.5 (bs, 1H), 11.8 (bs, 1H).

### Example 64

### (2R,4S,4aS)-rel-9,10-Difluoro-2,4-dimethyl-2′,4′,6′-trioxo-N-(tetrahydrofuran-3-yl)-1,1′,2,3′,4,4′,4a,6′-octahydro-2′H,6H-spiro[1,4-oxazino[4,3-a]quinoline-5,5′-pyrimidine]-8-carboxamide

Starting material: Tetrahydrofuran-3-amine.

MS (ES) MH⁺: 479.2 for C₂₂H₂₄F₂N₄O₆

¹H NMR (400 MHz, DMSO-d₆) δ: 0.9 (d, 3H), 1.1 (d, 3H), 1.8 (m, 1H), 2.1 (m, 1H), 2.8 (d, 1H), 3.05 (t, 1H), 3.5 (m, 2H), 3.6-3.8 (m, 6H), 4.02 (d, 1H), 4.4 (m, 1H), 7.04 (d, 1H), 8.1 (d, 1H), 11.5 (s, 1H), 11.8 (s, 1H).

### Example 65

### (2R,4S,4aS)-rel-9,10-Difluoro-2,4-dimethyl-2′,4′,6′-trioxo-N-(tetrahydro-2H-pyran-3-yl)-1,1′,2,3′,4,4′,4a,6′-octahydro-2′H,6H-spiro[1,4-oxazino[4,3-a]quinoline-5,5′-pyrimidine]-8-carboxamide

Starting material: 3-Aminotetrahydropyran.

MS (ES) MH⁺: 492.2 for C₂₃H₂₆F₂N₄O₆

¹H NMR (400 MHz, DMSO-d₆) δ: 0.9 (d, 3H), 1.1 (d, 3H), 1.7-1.8 (m, 3H), 2.5(m, 1H), 2.9 (d, 1H), 3.1 (t, 1H), 3.2 (bs, 2H), 3.5 (d, 1H), 3.6 (t, 1H), 3.7 (m, 3H), 3.9 (d, 2H), 4.1 (d, 1H); 7.1 (d, 1H), 8.3 (bs, 1H), 9.1 (bs, 2H), 11.5 (bs, 1H), 11.9 (bs, 1H).

### Example 66

### (2R,4S,4aS)-rel-9,10-Difluoro-2,4-dimethyl-2′,4′,6′-trioxo-N-(tetrahydro-2H-pyran-4-yl)-1,1′,2,3′,4,4′,4a,6′-octahydro-2′H,6H-spiro[1,4-oxazino[4,3-a]quinoline-5,5′-pyrimidine]-8-carboxamide

Starting material: Tetrahydropyran-4-amine.

MS (ES) MH⁺: 493.2 for C₂₃H₂₆F₂N₄O₆

¹H NMR (400 MHz, DMSO-d₆) δ: 0.9 (d, 3H), 1.1 (d, 3H),1.5 (m, 2H), 1.8 (m, 2H), 2.9 (d, 1H), 3.1 (t, 1H), 3.5 (m, 2H), 3.6 (d, 1H), 3.7 (m, 1H), 3.8 (m, 1H), 3.9 (m, 3H), 4.1 (d, 1H), 7.1 (d, 1H), 7.9 (d, 1H), 11.5 (s, 1H), 11.8 (s, 1H).

### Example 67

### tert-Butyl 4-({[(2R,4S,4aS)-rel-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]carbonyl}amino)piperidine-1-carboxylate

Starting material: tert-Butyl 4-aminopiperidine-1-carboxylate.

MS (ES) MH⁻: 590.2 for C₂₈H₃₅F₂N₅O₇

¹H NMR (400 MHz, DMSO-d₆) δ: 0.9(d, 3H), 1.1(d, 3H), 1.3(s, 9H), 1.7(d, 2H), 2.8(d, 2H), 3.0(t, 1H), 3.4(d, 1H), 3.6(t, 1H), 3.7(m, 1H), 3.9(m, 1H), 4.0(d, 1H), 7.0(s, 1H), 7.8(bs, 1H).

### Example 68

### (2R,4S,4aS)-rel-9,10-Difluoro-2,4-dimethyl-2′,4′,6′-trioxo-N-[1-(pyridin-2-yl)piperidin-4-yl]-1,1′,2,3′,4,4′,4a,6′-octahydro-2′H,6H-spiro[1,4-oxazino[4,3-a]quinoline-5,5′-pyrimidine]-8-carboxamide

Starting material: 1-(2-Pyridinyl)-4-piperidinamine

MS (ES) MH⁺: 569.2 for C₂₈H₃₀F₂N₆O₅

¹H NMR (400 MHz, DMSO-d₆) δ: 0.8(d, 3H), 1.1(d, 3H), 1.8(d, 2H), 2.8(d, 2H), 2.9(t, 2H), 3.0(t, 1H), 3.4(d, 1H), 3.6(m, 1H), 3.7(t, 1H), 3.8(d, 1H), 4.0(d, 2H), 4.2(d, 2H), 6.5(t, 1H), 6.8(d, 1H), 7.0(bs, 1H), 7.5(t, 1H), 7.8(d, 1H), 8.0(bs, 1H).

### Example 69

### (2R,4S,4aS)-rel-9,10-Difluoro-2,4-dimethyl-N-(1-methylpiperidin-3-yl)-2′,4′,6′-trioxo-1,1′,2,3′,4,4′,4a,6′-octahydro-2′H,6H-spiro[1,4-oxazino[4,3-a]quinoline-5,5′-pyrimidine]-8-carboxamide

Starting material: 3-Amino-1-methylpiperidine.

MS (ES) MH⁺: 506.2 for C₂₄H₂₉F₂N₅O₅

¹H NMR (400 MHz, CD₃OD) δ: 1.0 (d, 3H), 1.2 (d, 3H), 1.7-1.8 (m, 2H), 2.1 (m, 2H); 2.8 (m, 1H), 2.9(s, 3H), 3.1 (t, 2H); 3.2 (bs, 1H); 3.5 (d, 1H); 3.6 (d, 2H); 3.7 (m, 1H); 3.9 (d, 1H) 4.2 (m, 1H), 7.1 (d, 1H).

### Example 70

### (2R,4S,4aS)-rel-9,10-Difluoro-2,4-dimethyl-2′,4′,6′-trioxo-N-(pyrrolidin-3-yl)-1,1′,2,3′,4,4′,4a,6′-octahydro-2′H,6H-spiro[1,4-oxazino[4,3-a]quinoline-5,5′-pyrimidine]-8-carboxamide

A mixture of tert-butyl 3-({[(2R,4S,4aS)-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]carbonyl}amino)pyrrolidine-1-carboxylate (Example 63, 100 mg) and HCl in anhydrous ether (2 mL) was stirred at room temperature for 1 hour, during which time TLC showed the deprotection of the Boc group. The reaction mixture was concentrated under reduced pressure. The residue thus obtained was triturated with ether to give the title compound as a yellow solid. Yield: 50 mg (60%)

MS (ESP) MH⁺: 476.2 for C₂₂H₂₅F₂N₅O₅

¹H NMR (400 MHz, DMSO-d₆) δ: 0.9 (d, 3H), 1.1 (d, 3H), 1.9 (m, 1H), 2.2 (m, 1H), 2.9 (d, 1H), 3.1 (m, 1H), 3.2 (m, 1H), 3.6 (m, 1H), 3.7 (m, 2H), 3.8 (d, 1H), 4.1 (d, 1H), 4.5 (m, 1H), 7.1 (d, 1H), 8.3 (bs, 1H), 9.1 (bs, 2H), 11.5 (bs, 1H), 11.9 (bs, 1H).

### Example 71

### (2R,4S,4aS)-rel-9,10-Difluoro-2,4-dimethyl-2′,4′,6′-trioxo-N-(piperidin-4-yl)-1,1′,2,3′,4,4′,4a,6′-octahydro-2′H,6H-spiro[1,4-oxazino[4,3-a]quinoline-5 ,5′-pyrimidine]-8-carboxamide

The title compound was synthesized from tert-Butyl 4-({[(2R,4S,4aS)-9,10-difluoro-2,4-dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]carbonyl}amino)piperidine-1-carboxylate (Example 67) using a method similar to the one described for the synthesis of Example 70.

MS (ES) MH⁺: 492.2 for C₂₃H₂₇F₂N₅O₅

¹H NMR (400 MHz, DMSO-d₆) δ: 0.9 (d, 3H), 1.1 (d, 3H), 1.7 (m, 2H), 1.9 (d, 3.0 (m, 4H), 3.3 (m, 2H), 3.5 (d, 2H), 3.6 (m, 1H), 3.8 (m, 1H), 3.9 (d, 1H), 4.1 (m, 1H); 7.0 (d, 1H); 8.1 (d, 1H), 8.6 (d, 2H) 11.5 (bs, 1H), 11.8 (bs, 1H).

### Example 72

### 1-[(2R,4S,4aS)-rel-2,4-Dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]-3-ethylurea

To a solution of (2R,4S,4aS)-8-amino-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione (Intermediate 34, 344 mg, 1 mmol) in dry THF (2 mL) was added triethylamine (0.42 ml, 3 mmol) followed by ethyl isocyanate 0.095 ml, 1.2 mmol). The reaction mixture was stirred at room temperature for 12 hours. Solvents were evaporated and the residue purified over a silica gel (230-400) column using a gradient of ethyl acetate in petroleum ether to give 70 mg of the title product.

MS(ES) MH⁺: 416.2 for C₂₀H₂₅N₅O₅

¹H NMR (400 MHz, DMSO-d₆) δ: 0.89 (d, 3H), 1.0 (t, 3H),1.24 (d, 3H), 2.66 (m, 1H), 2.89 (d, 1H), 3.04(m, 2H), 3.14 (d, 1H), 3.46 (m, 1H), 3.59 (m, 2H), 3.87 (d, 1H), 5.92 (m, 1H), 6.70 (d, 1H), 6.92 (s, 1H), 7.04 (d, 1H), 8.02 (s, 1H), 11.41 (s, 1H), 11.65 (s, 1H).

### Example 73

### 1-[(2R,4S,4aS)-rel-2,4-Dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]-3-phenylurea

The title compound was synthesized from (2R,4S,4aS)-8-amino-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione (Intermediate 34) and phenyl isocyanate using a method similar to the one described for the synthesis of Example 72.

MS(ES) MH⁺: 464.4 for C₂₄H₂₅N₅O₅

¹H NMR (400 MHz , DMSO-d₆): 0.9 (d, 3H), 1.1 (d, 3H), 2.7-2.8 (m, 1H), 3.0 (d, 1H), 3.2 (d, 1H), 3.5-3.9 (m, 3H), 3.94 (d, 1H), 6.8 (d, 1H), 6.9-6.95 (m, 1H), 7.0 (s, 1H), 7.1 (d, 1H), 7.25 (m, 2H), 7.4 (d, 2H), 8.2 (s, 1H), 8.8 (s, 1H), 11.4 (s, 1H), 11.7 (s, 1H).

### Example 74

### N-[(2R,4S,4aS)-rel-2,4-Dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]piperidine-1-carboxamide

To a solution of (2R,4S,4aS)-8-amino-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione (Intermediate, 34, 500 mg, 1.45 mmol) in anhydrous THF (10 mL) at 0° C. was added triethylamine (440 mg, 4.35 mmol), followed by 1-piperidinecarbonyl chloride (214 mg, 1.45 mmol), and the mixture was stirred at room temperature for 12 hours. The reaction mixture was quenched with HCl (1N, 50 ml), extracted with ethyl acetate (2×20 mL). The combined organic phases were dried over anhydrous sodium sulfate and concentrated under reduced pressure. The residue thus obtained was washed with ether to give 200 mg of the title product. (Yield: 30%).

MS(ES) MH⁺: 456.6 for C₂₃H₂₉N₅O₆

¹H NMR (400 MHz, DMSO-d₆) δ: 0.89 (d, 3H), 1.12 (d, 3H),1.45 (d, 4H), 1.55 (d, 2H), 2.68 (t, 1H), 2.90 (d, 1H), 3.12 (d, 1H), 3.28 (d, 2H), 3.53-3.79 (m, 4H), 3.89 (d, 1H), 6.71 (d, 1H), 6.97 (d, 1H), 7.09 (q, 1H), 8.08 (s, 1H), 11.40 (s, 1H), 11.65 (s, 1H).

Examples 75 to 77 were synthesized from (2R,4S,4aS)-8-amino-2,4-dimethyl-1,2,4,4a-tetrahydro-2′H,6H-spiro[1,4-oxazino[4,3-a]quinoline-5,5′-pyrimidine]-2′,4′,6′(1′H,3+H)-trione (Intermediate 34) and the starting materials, using a method similar to the one described for the synthesis of Example 74.

### Example 75

### N-[(2R,4S ,4aS)-rel-2,4-Dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]morpholine-4-sulfonamide

Starting material: 4-Morpholinesulfonyl chloride.

MS(ES) MH⁺: 494.2 for C₂₁H₂₇N₅O₇S

¹H NMR (400 MHz, DMSO-d₆) δ: 0.9(d, 3H), 1.1 (d, 3H), 2.5 (m, 2H), 3.0 (m, 4H), 3.2-3.3 (m, 6H), 3.5 (m, 3H), 3.6 (d, 1H), 3.95 (d, 1H), 6.8 (m, 2H), 6.9 (q, 1H), 9.4 (s, 1H), 11.4 (s, 1H), 11.7 (s, 1H).

### Example 76

### N-[(2R,4S,4aS)-rel-2,4-Dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]-2-oxoimidazolidine-1-carboxamide

Starting material: 1-(Chlorocarbonyl)-2-imidazolidinone.

MS(ES) MH⁺: 457.1 for C₂₁H₂₄N₆O₆

¹H NMR (400 MHz, DMSO-d₆) δ: 0.9 (d, 3H), 1.2 (d, 3H), 2.7 (m, 1H), 2.9 (m, 1H), 3.1 (d, 1H), 3.3-3.6 (m, 3H), 3.9 (d, 1H), 6.7 (dd, 1H), 6.95 (d, 1H), 7.1 (q, 1H), 8.3 (s, 1H), 11.4 (s, 1H), 11.65 (s, 1H).

### Example 77

### 3-[(2R,4S,4aS)-rel-2,4-Dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]-1,1-dimethylurea

Starting material: Dimethylcarbmoyl chloride.

MS(ES) MH⁺: 416.2 for C₂₀H₂₅N₅O₅

¹H NMR (400 MHz, DMSO-d₆) δ: 0.9 (d, 3H), 1.1 (d, 3H), 2.5 (t, 1H), 2.9 (d, 1H), 3.2 (d, 1H), 3.3 (m, 2H), 3.5 (m, 3H), 3.8 (t, 2H), 3.9 (d, 1H), 6.8 (d, 1H), 7.0 (d, 1H), 7.2 (q, 1H), 7.7 (s, 1H), 10.1 (s, 1H), 11.4 (s, 1H), 11.7 (s, 1H)

### Example 78

### 1-[(2R,4S,4aS)-rel-2,4-Dimethyl-2′,4′,6′-trioxo-1,1′,2,3′,4,4′,4a,6′-octahydro-2′H,6H-spiro[1,4-oxazino[4,3-a]quinoline-5,5′-pyrimidin]-8-yl]-3-methylurea

To a solution of (2R,45,4a5)-8-amino-2,4-dimethyl-1,2,4,4a-tetrahydro-2′H,6H-spiro[1,4-oxazino[4,3-a]quinoline-5,5′-pyrimidine]-2′,4′,6′(1′H,3′H)-trione (Intermediate 34, 250 mg, 0.74 mmol) in anhydrous THF (20 mL) at 0° C. was added triethylamine (2.5 g, 0.024 mol) followed by methylphenoxycarbamate (0.66 mmol) and the mixture was refluxed for 48 hours. Methyl phenoxy carbamate was prepared by stirring methyl amine (1 eq.) and phenylchloroformate (1 eq.) in DCM at −30° C. to room temperature for 4 hours, and was purified by column chromatography. The reaction mixture was quenched with HCl (1N, 30 ml) and extracted with ethyl acetate (5×20 mL). The organic phase was dried over anhydrous sodium sulphate and was concentrated under reduced pressure. The residue thus obtained was washed with ether and further purified by preparative TLC to give the title compound. Yield: 20 mg (70%).

MS(ES) MH⁺: 402.2 for C₁₉H₂₃N₅O₅

¹H NMR (400 MHz, DMSO-d₆) δ: 0.9 (d, 3H), 1.1 (d, 3H), 2.5 (d, 3H), 2.9 (d, 1H), 3.1 (d, 1H), 3.5 (d, 1H), 3.55 (m, 2H), 3.85 (d, 1H), 5.8 (m, 1H), 6.7 (d, 1H), 6.9 (s, 1H), 7.1 (d, 1H), 11.7 (bs, 2H).

### Example 79

### (2R,4S,4aS)-9,10-Difluoro-2,4-dimethyl-8-(pyrazin-2-ylethynyl)-2,4,4a,6-tetrahydro-1H,1′H-spiro[[1,4]oxazino[4,3-a]quinoline-5,5′-pyrimidine]-2′,4′,6′(3′H)-trione

A reaction flask was charged with (2R,4S,4aS)-9,10-difluoro-8-iodo-2,4-dimethyl-2,4,4a,6-tetrahydro-1H,1′H-spiro[[1,4]oxazino[4,3-a]quinoline-5,5′-pyrimidine]-2′,4′,6-(Intermediate 89(b), 350 mg, 0.71 mmol), copper(I) iodide (6.78 mg, 0.04 mmol) and dichlorobis(triphenylphosphne)palladium (II) (25.01 mg, 0.04 mmol) in acetonitrile (3 ml) under N₂. The flask was degassed and backfilled 3 times with a balloon containing a 50:50 mixture of Argon/H₂. TEA (0.794 ml, 5.70 mmol), which had been degassed by bubbling Ar through for 20 minutes, was added. A solution of 2-ethynylpyrazine (119 mg, 1.14 mmol) in 1 ml CH₃CN that had been degassed by bubbling Ar through for 5 minutes, was added. The reaction mixture was heated to 90° C. (external temperature) under the N₂/H₂ balloon atmosphere for 45 minutes. The mixture was diluted with EtOAc and washed with water and brine. The combined aqueous layers were twice more extracted with EtOAc, which was washed with brine. The combined EtOAc extracts were dried and concentrated, and the residue was chromatographed on silica gel (100% CH₂Cl₂, followed by gradient elution to 100% EtOAc), to afford the title product as a yellow solid.

MS (MH⁺): 468 for C₂₃H₁₉F₂N₅O₄

¹H NMR (300MHz, DMSO-d₆) δ: 0.9 (d, 3H), 1.1 (d, 3H), 2.9 (d, 1H), 3.1 (m, 1H), 3.5 (d, 1H), 3.7-3.8 (m, 1H), 3.9 (m, 1H), 4.1 (d, 1H), 7.1 (d, 1H), 8.6 (s, 1H), 8.7 (s, 1H), 8.8 (s, 1H), 11.6 (s, 1H), 11.9 (s, 1H).

### Example 80

### (2S,4R,4aR)-9,10-Difluoro-2,4-dimethyl-8-(pyrazin-2-ylethynyl)-2,4,4a,6-tetrahydro-1H,1′H-spiro[[1,4]oxazino[4,3-a]quinoline-5,5′-pyrimidine]-2′,4′,6′(3′H)-trione

The title compound was synthesized from (2S,4R,4aR)-9,10-difluoro-8-iodo-2,4-dimethyl-2,4,4a,6-tetrahydro-1H,1′H-spiro[[1,4]oxazino[4,3-a]quinoline-5,5′-pyrimidine]-2′,4′,6′(3′H)-trione (Intermediate 89(a), 100 mg, 0.20 mmol) using a procedure similar to the one described for the synthesis of Example 79, providing 37 mg of title compound.

MS (MH⁺): 468 for C₂₃H₁₉F₂N₅O₄

¹H NMR (300 MHz, DMSO-d₆) δ: 0.9 (d, 3H), 1.1 (d, 3H), 2.9 (d, 1H), 3.1 (m, 1H), 3.5 (d, 1H), 3.7-3.8 (m, 1H), 3.9 (m, 1H), 4.1 (d, 1H), 7.1 (d, 1H), 8.6 (s, 1H), 8.7 (s, 1H), 8.8 (s, 1H), 11.6 (s, 1H), 11.9 (s, 1H).

1. A compound of Formula (I): 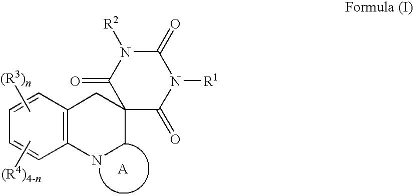
or a pharmaceutically acceptable salt thereof, wherein:
R¹ is selected from H, C₁₋₆alkyl, carbocyclyl, heterocyclyl, —C(O)—H, —C(O)—R^{1b}, —C(O)₂R^{1c}, —C(O)—N(R^{1a})₂, —S(O)—R^{1b}, —S(O)₂—R^{1b}, —S(O)₂—N(R^{1a})₂, —C(R^{1a})═N—R^{1a}, and —C(R^{1a})═N—OR^{1a}, wherein said C₁₋₆alkyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R¹⁰*;
R^{1a} in each occurrence is independently selected from H, C₁₋₆-alkyl, carbocyclyl, and heterocyclyl, wherein said C₁₋₆alkyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R¹⁰*;
R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R¹⁰*;
R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein if said heterocyclyl contains an —NH— moiety, that nitrogen in each occurrence is optionally and independently substituted with R¹⁰*;
R² is selected from H, C₁₋₆alkyl, carbocyclyl, heterocyclyl, —C(O)—H, —C(O)—R^{2b}, —C(O)₂R^{2c}, —C(O)—N(R^{2a})₂, —S(O)—R^{2b}, —S(O)₂—R^{2b}, —S(O)₂—N(R^{2a})₂, —C(R^{2a})═N—R^{2a}, and —C(R^{2a})═N—OR^{2a}, wherein said C₁₋₆alkyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R²⁰*;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R²⁰*;
R^{2b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R²⁰*;
R^{2c} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein if said heterocyclyl contains an —NH— moiety, that nitrogen in each occurrence is optionally and independently substituted with R²⁰*;
R³ in each occurrence is independently selected from —X—R⁵, —W—R⁶, —C(O)—N(R^{3a})—S(O)₂—R^{3b}, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})₂, —C(N(R^{3a})₂)═N—R^{3y}, —C(N(R^{3a})₂)═N—OR^{3y}, —C(N(R^{3a})₂)═N—C(O)—R^{3b}, —C(N(R^{3a})₂)═N—S(O)₂—R^{3b}, —C(N(R^{3a})₂)═N—CN, —N═C(R^{3y})₂, —N(R^{3a})—S(O)₂—N(R^{3y})₂, —N(R^{3a})—N(R^{3y})₂, —N(R^{3a})—C(O)—N(R^{3y})₂, —N(R^{3a})—C(O)—N(R^{3a})—S(O)₂—R^{3b}, —N(R^{3a})—C(R^{3a})═N(R^{3y}), —N(R^{3a})—C(R^{3a})═N—OR^{3y}, —N(R^{3a})—C(R^{3a})═N—C(O)—R^{3b}, —N(R^{3a})—C(R^{3a})═N—S(O)₂R^{3b}, —N(R^{3a})—C(R^{3a})═N—CN, —N(R^{3a})—C(N(R^{3a})₂)═N—R^{3y}, —N(R^{3a})—C(N(R^{3a})₂)═N—OR^{3y}, —N(R^{3a})—C(N(R^{3a})₂)═N—C(O)—R^{3b}, —N(R^{3a})—C(N(R^{3a})₂)═N—S(O)₂—R^{3b}, —N(R^{3a})—C(N(R^{3a})₂)═N—CN, —O—C(O)—R^{3b}, and —Si(R^{3b})₃;
R^{3a} and R^{3y} in each occurrence are independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein if said heterocyclyl contains an —NH— moiety, that nitrogen in each occurrence is optionally and independently substituted with R³⁰*;
R^{3b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein if said heterocyclyl contains an —NH— moiety, that nitrogen in each occurrence is optionally and independently substituted with R³⁰*;
R⁴ in each occurrence is independently selected from H, halo, —CN, C₁₋₆alkyl, C₂₋₆alkenyl, C₂₋₆alkynyl, carbocyclyl, heterocyclyl, —OR^{4d}, —SR^{4d}, —N(R^{4d})₂, —N(R^{4e})—C(O)—R^{4e}, —NO₂, —C(O)—H, —C(O)—R⁴e, —C(O)₂R^{4d}, —C(O)N(R^{4a})(R^{4d}), —O—C(O)—N(R^{4a})(R^{4d}), —N(R^{4a})—C(O)₂R^{4d}, —S(O)—R^{4e}, —S(O)₂—R^{4e}, —S(O)₂—N(R^{4a})(R^{4d}), —N(R^{4a})—S(O)₂—R^{4e}, and —C(R^{4e})═N—OR^{4d}, wherein said C₁₋₆alkyl, C₂₋₆alkenyl, and C₂₋₆alkynyl in each occurrence are optionally and independently substituted with one or more R^{40x}, and wherein said carbocyclyl and heterocyclyl in each occurrence are optionally and independently substituted on carbon with one or more R⁴⁰, and wherein if said heterocyclyl contains an —NH— moiety, that nitrogen in each occurrence is optionally and independently substituted with R⁴⁰*;
R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein if said heterocyclyl contains an —NH— moiety, that nitrogen in each occurrence is optionally and independently substituted with R⁴⁰*;
R^{4d} in each occurrence is independently selected from H, C₁₋₆alkyl, carbocyclyl, and aromatic heterocyclyl, wherein said C₁₋₆alkyl, carbocyclyl, and aromatic heterocyclyl in each occurrence are optionally and independently substituted on carbon with one or more R⁴⁰, and wherein if said aromatic heterocyclyl contains an —NH— moiety, that nitrogen in each occurrence is optionally and independently substituted with R⁴⁰*;
R^{4e} in each occurrence is selected from C₁₋₆alkyl, C₂₋₆alkenyl, C₂₋₆alkynyl, carbocyclyl, and aromatic heterocyclyl, wherein said C₁₋₆alkyl, C₂₋₆alkenyl, C₂₋₆alkynyl, carbocyclyl, and aromatic heterocyclyl in each occurrence are optionally and independently substituted on carbon with one or more R⁴⁰, and wherein if said aromatic heterocyclyl contains an —NH— moiety, that nitrogen in each occurrence is optionally and independently substituted with R⁴⁰*;
R⁵ is selected from heterocyclyl and —Si(R^{5b})₃, wherein said heterocyclyl is optionally substituted on carbon with one or more R⁵⁰, and wherein if said heterocyclyl contains an —NH— moiety, that nitrogen in each occurrence is optionally and independently substituted with R⁵⁰*;
R^{5b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein if said heterocyclyl contains an —NH— moiety, that nitrogen in each occurrence is optionally and independently substituted with R⁵⁰*;
R⁶ is non-aromatic heterocyclyl, wherein said non-aromatic heterocyclyl is optionally substituted on carbon with one or more R⁶⁰, and wherein if said non-aromatic heterocyclyl contains an —NH— moiety, that nitrogen in each occurrence is optionally and independently substituted with R⁶⁰*;
R⁷ is selected from halo, —CN, C₁₋₆alkyl, C₂₋₆alkenyl, C₂₋₆alkynyl, carbocyclyl, heterocyclyl, —OR^{7a}, —SR^{7a}, —N(R^{7a})₂, —N(R^{7a})—C(O)—R^{7b}, —N(R^{7a})—N(R^{7a})₂, —NO₂, —C(O)—H, —C(O)R^{7b}, —C(O)₂R^{7a}, —C(O)—N(R^{7a})₂, —O—C(O)—N(R^{7a})₂, —N(R^{7a})—C(O)₂R^{7a}, —N(R^{7a})—C(O)—N(R^{7a})₂, —O—C(O)—R^{7b}, —S(O)—R^{7b}, —S(O)₂—R^{7b}, —S(O)₂—N(R^{7a})₂, —N(R^{7a})—S(O)₂—R^{7b}, —C(R^{7a})═N—R^{7a}, and —C(R^{7a})═N—OR^{7a}, wherein said C₁₋₆alkyl, C₂₋₆alkenyl, C₂₋₆alkynyl, carbocyclyl, and heterocyclyl are optionally substituted on carbon with one or more R⁷⁰, and wherein if said heterocyclyl contains an —NH— moiety, that nitrogen in each occurrence is optionally and independently substituted with R⁷⁰*;
R⁷* in each occurrence is independently selected from C₁₋₆alkyl, carbocyclyl, heterocyclyl, —C(O)—H, —C(O)—R^{7b}, —C(O)₂R^{7c}, —C(O)—N(R^{7a})₂, —S(O)—R^{7b}, —S(O)₂—R^{7b}, —S(O)₂—N(R^{7a})₂, —C(R^{7a})═N—R^{7a}, and —C(R^{7a})═N—OR^{7a}, wherein said C₁₋₆alkyl, carbocyclyl, and heterocyclyl in each occurrence are optionally and independently substituted on carbon with one or more R⁷⁰, and wherein if said heterocyclyl contains an —NH— moiety, that nitrogen in each occurrence is optionally and independently substituted with R⁷⁰*;
R^{7a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein if said heterocyclyl contains an —NH— moiety, that nitrogen in each occurrence is optionally and independently substituted with R⁷⁰*;
R^{7b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁷⁰, and wherein if said heterocyclyl contains an —NH— moiety, that nitrogen in each occurrence is optionally and independently substituted with R⁷⁰*;
R^{7b} in each occurrence may be independently selected from C₁₋₆alkyl, carbocyclyl, and heterocyclyl, wherein said C₁₋₆alkyl, carbocyclyl, and heterocyclyl in each occurrence may be optionally and independently substituted on carbon with one or more R⁷⁰, and wherein any —NH— moiety of said heterocyclyl may be optionally substituted with R⁷⁰*;
R¹⁰ in each occurrence is independently selected from halo, —CN, C₁₋₆alkyl, C₂₋₆alkenyl, C₂₋₆alkynyl, carbocyclyl, heterocyclyl, OR^{10a}, —SR^{10a}, —N(R^{10a})₂, —N(R^{10a})—C(O)—R^{10b}, —N(R^{10a})—N(R^{10a})₂, —NO₂, —C(O)—H, —C(O)—R^{10b}, —C(O)₂R^{10a}, —C(O)—N(R^{10a})₂, —O—C(O)—N(R^{10a})₂, —N(R^{10a})—C(O)₂R^{10a}, —N(R^{10a})—C(O)—N(R^{10a})₂, —O—C(O)—R^{10b}, —S(O)—R^{10b}, —S(O)₂—R^{10b}, —S(O)₂—N(R^{10a})₂, —N(R^{10a})₂, —N(R^{10a})—S(O)₂—R^{10b}, —C(R^{10a})═N—R^{10a}, and —C(R^{10a})═N—OR^{10a}, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a}*;
R¹⁰* in each occurrence is independently selected from C₁₋₆alkyl, carbocyclyl, heterocyclyl, —C(O)—H, —C(O)—R^{10b}, —C(O)₂R^{10c}, —C(O)—N(R^{10a})₂, —S(O)R^{10b}, —S(O)₂R^{10b}, —S(O)₂—N(R^{10a})₂, —C(R^{10a})═N—R^{10a}, and —C(R^{10a})═N—OR^{10a}, wherein said C₁₋₆alkyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a}*;
R^{10a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a}*;
R^{10b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a}*;
R^{10c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein if said heterocyclyl contains an —NH— moiety, that nitrogen in each occurrence is optionally and independently substituted with R^{a}*;
R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})—C(O)—R^{20b}, —N(R^{20a})—N(R^{20a})₂, —NO₂, —C(O)—H, —C(O)—R^{20b}, —C(O)₂R^{20a}, —C(O)—N(R^{20a})₂, —O—C(O)—N(R^{20a})₂, —N(R^{20a})—C(O)₂R^{20a}, —N(R^{20a})—C(O)—N(R^{20a})₂, —O—C(O)—R^{20b}, —S(O)—R^{20b}, —S(O)₂—R^{20b}, —S(O)₂—N(R^{20a})₂, —N(R^{20a})—S(O)₂—R^{20b}, —C(R^{20a})═N—R^{20a}, and —C(R^{20a})═N—OR^{20a}, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b}*;
R²⁰* in each occurrence is independently selected from C₁₋₆alkyl, carbocyclyl, heterocyclyl, —C(O)—H, —C(O)—R^{20b}, —C(O)₂R^{20c}, —C(O)—N(R^{20a})₂, —S(O)—R^{20b}, —S(O)₂—R^{20b}, —S(O)₂—N(R^{20a})₂, —C(R^{20a})═N—R^{20a}, and —C(R^{20a})═N—OR^{20a}, wherein said C₁₋₆alkyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b}*;
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b}*,
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b}*;
R^{20c} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein if said heterocyclyl contains an —NH— moiety, that nitrogen in each occurrence is optionally and independently substituted with R^{b}*;
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})—C(O)—R^{30b}, —N(R^{30a})—N(R^{30a})₂, —NO₂, —C(O)H, —C(O)—R^{30b}, —C(O)₂R^{30a}, —C(O)—N(R^{30a})₂, —O—C(O)—N(R^{30a})₂, —N(R^{30a})—C(O)₂R^{10a}, —N(R^{30a})—C(O)—N(R^{30a})₂, —O—C(O)—R^{30b}, —S(O)—R^{30b}, —S(O)₂—R^{30b}, —S(O)₂—N(R^{30a})₂, —N(R^{30a})—S(O)₂—R^{30b}, —Si(R^{30b})₃, —C(R^{30a})═N—R^{30a}, and —C(R^{30a})═N—OR^{30a}, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c}*;
R^{30*} in each occurrence is independently selected from C₁₋₆alkyl, carbocyclyl, heterocyclyl, —C(O)—H, —C(O)—R^{30b}, —C(O)₂R^{30c}, —C(O)—N(R^{30a})₂, —S(O)—R^{30b}, —S(O)₂—R^{30b}, —S(O)₂—N(R^{30a})₂, —C(R^{30a})═N—R^{30a}, and —C(R^{30a})═N—OR^{30a}, wherein said C₁₋₆alkyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c}*;
R^{30a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c}*;
R^{30b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c}*;
R^{30c} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein if said heterocyclyl contains an —NH— moiety, that nitrogen in each occurrence is optionally and independently substituted with R^{c}*;
R⁴⁰ in each occurrence is independently selected from halo, —CN, C₁₋₆alkyl, C₂₋₆alkenyl, C₂₋₆alkynyl, carbocyclyl, heterocyclyl, —OR⁴⁰a, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R^{40a})₂, —N(R^{40a})—S(O)₂—R^{40b}, —C(R^{40a})═N—R^{40a}, and —C(R^{40a})═N—OR^{40a}, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d}*;
R⁴⁰* in each occurrence is independently selected from C₁₋₆alkyl, carbocyclyl, heterocyclyl, —C(O)—H, —C(O)—R^{40b}, —C(O)₂R^{40c}, —C(O)—N(R^{40a})₂, —S(O)—R^{40b}, —S(O)₂—R^{40b}, —S(O)₂—N(R^{40a})₂, —C(R^{40a})═N—R^{40a}, and —C(R^{40a})═N—OR^{40a}, wherein said C₁₋₆alkyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d}*;
R^{40a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d}*;
R^{40b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d}*;
R^{40c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d}*;
R^{40x} in each occurrence is independently selected from halo, —CN, C₁₋₆alkyl, C₂₋₆alkenyl, C₂₋₆alkynyl, carbocyclyl, —OR^{40a}, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R⁴⁰)₂, —N(R^{40a})—S(O)₂—R^{40b}, —C(R^{40a})═N+R^{40a}, and heterocyclyl in each occurrence are optionally and independently substituted on carbon with one or more R^{d}, and wherein if said heterocyclyl contains an —NH— moiety, that nitrogen in each occurrence is optionally and independently substituted with R^{d}*;
R⁵⁰ in each occurrence is independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})—C(O)—R^{50b}, —N(R^{50a})—N(R^{50a})₂, —NO₂, —C(O)—H, —C(O)—R^{50b}, —C(O)₂R^{50a}, —C(O)—N(R^{50a})₂, —O—C(O)—N(R^{50a})₂, —N(R^{50a})—C(O)₂R^{50a}, —N(R^{50a})—C(O)—N(R^{50a})₂, —O—C(O)—R^{50b}, —S(O)—R^{50b}, —S(O)₂—R^{50b}, —S(O)₂—N(R^{50a})₂, —N(R^{50a})—S(O)₂—R^{50b}, —Si(R^{50b})₃, —C(R^{50a})═N(R^{50a}), and —C(R^{50a})═N(OR^{50a}), wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e}*;
R⁵⁰* in each occurrence is independently selected from C₁₋₆alkyl, carbocyclyl, heterocyclyl, —C(O)—H, —C(O)—R^{50b}, —C(O)₂R^{50c}, —C(O)—N(R^{50a})₂, —S(O)—R^{50b}, —S(O)₂—R^{50b}, —S(O)₂—N(R^{50a})₂, —C(R^{50a})═N—R^{50a}, and —C(R^{50a})═N—OR^{50a}, wherein said C₁₋₆alkyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e}*;
R^{50a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e}*;
R^{50b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e}*;
R^{50c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein if said heterocyclyl contains an —NH— moiety, that nitrogen in each occurrence is optionally and independently substituted with R^{e}*;
R⁶⁰ in each occurrence is independently selected from halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})—C(O)—R^{60b}, —N(R^{60a})—N(R^{60a})₂, —NO₂, —C(O)—H, —C(O)—R^{60b}, —C(O)₂R^{60a}, —C(O)—N(R^{60a})₂, —O—C(O)—N(R^{60a})₂, —N(R^{60a})—C(O)₂R^{60a}, —N(R^{60a})—C(O)—N(R^{60a})₂, —O—C(O)—R^{60b}, —S(O)—R^{60b}, —S(O)₂—R^{60b}, —S(O)₂—N(R^{60a})₂, —N(R^{60a})—S(O)₂—R^{60b}, —C(R^{60a})═N—R^{60a}, and —C(R^{60a})═N—OR^{60a}, wherein said C₁₋₆alkyl, C₂₋₆alkenyl, C₂₋₆alkynyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f}*;
R⁶⁰* in each occurrence is independently selected from C₁₋₆alkyl, carbocyclyl, heterocyclyl, —C(O)—H, —C(O)—R^{60b}, —C(O)₂R^{60c}, —C(O)—N(R^{60a})₂, —S(O)—R^{60b}, —S(O)₂—R^{60b}, —S(O)₂—N(R^{60a})₂, —C(R^{60a})═N—R^{60a}, and —C(R^{60a})═N—OR^{60a}, wherein said C₁₋₆alkyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f}*;
R^{60a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{f}*;
R^{60b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f}*;
R^{60c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{f}, and wherein if said heterocyclyl contains an —NH— moiety, that nitrogen in each occurrence is optionally and independently substituted with R^{f}*;
R⁷⁰ in each occurrence is independently selected from halo, —CN, C₁₋₆alkyl, C₂₋₆alkenyl, C₂₋₆alkynyl, carbocyclyl, heterocyclyl, —OR^{70a}, —SR^{70a}, —N(R^{70a})₂, —N(R^{70a})—C(O)—R^{70b}, —N(R^{70a})—N(R^{70a})₂, —NO₂, —C(O)—H, —C(O)—R^{70b}, —C(O)₂R^{70a}, —C(O)—N(R^{70a})₂, —O—C(O)—N(R^{70a})₂, —N(R^{70a})—C(O)₂R^{70a}, —N(R^{70a})—C(O)—N(R^{70a})₂, —O—C(O)—R^{70b}, —S(O)—R^{70b}, —S(O)₂—R^{70b}, —S(O)₂—N(R^{70a})₂, —N(R^{70a})—S(O)₂—R^{70b}, —C(R^{70a})═N—R^{70a}, and —C(R^{70a})═N—OR^{70a}, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g}*;
R⁷⁰* in each occurrence is independently selected from C₁₋₆alkyl, carbocyclyl, heterocyclyl, —C(O)—H, —C(O)—R^{70b}, —C(O)₂R^{70c}, —C(O)—N(R^{70a})₂, —S(O)—R^{70b}, —S(O)₂—R^{70b}, —S(O)₂—N(R^{70a})₂, —C(R^{70a})═N—R^{70a}, and —C(R^{70a})═N—OR^{70a}, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g}*;
R^{70a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g}* ;
R^{70b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g}*;
R^{70c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein if said heterocyclyl contains an —NH— moiety, that nitrogen in each occurrence is optionally and independently substituted with R^{g}*;
R^{a}, R^{b}, R^{c}, R^{d}, R^{e}, R^{f}, and R^{g} in each occurrence are independently selected from halo, —CN, C₁₋₆alkyl, C₂₋₆alkenyl, C₂₋₆alkynyl, carbocyclyl, heterocyclyl, —OR^{m}, —0SR^{m}, —N(R^{m})₂, —N(R^{m})—C(O)—Rⁿ, —N(R^{m})—N(R^{m})₂, —NO₂, —C(O)—H, —C(O)—Rⁿ, —C(O)₂R^{m}, —C(O)—N(R^{m})₂, —O—C(O)—N(R^{m})₂, —N(R^{m})—C(O)₂R^{m}, —N(R^{m})—C(O)—N(R^{m})₂, —O—C(O)—Rⁿ, —S(O)—Rⁿ, —S(O)₂—Rⁿ, —S(O)₂—N(R^{m})₂, —N(R^{m})—S(O)₂—Rⁿ, —C(R^{m})═N—R^{m}, and —C(R^{m})═N—OR^{m};
R^{a}*, R^{b}*, R^{c}*, R^{d}, R^{e}*, R^{f}, and R^{g} in each occurrence are independently selected from carbocyclyl, heterocyclyl, —C(O)—H, —C(O)—Rⁿ, —C(O)₂R^{o}, —C(O)—N(R^{m})₂, —S(O)—Rⁿ, —S(O)₂—Rⁿ, —S(O)₂—N(R^{m})₂, —C(R^{m})═N—R^{m}, and —C(R^{m})═N—OR^{m};
R^{m} in each occurrence is independently selected from H, C₁₋₆alkyl, carbocyclyl, and heterocyclyl;
Rⁿ in each occurrence is independently selected from C₁₋₆alkyl, C₂₋₆alkenyl, C₂₋₆alkynyl, carbocyclyl, and heterocyclyl;
R^{o} in each occurrence is independently selected from C₁₋₆alkyl, carbocyclyl, and heterocyclyl;
W in each occurrence is independently selected from —O—, —S—, —N(R^{3a})—, —N(R^{3a})—C(O)—, —C(O)—, —C(O)₂—, —C(O)—N(R^{3a})—, —O—C(O)—N(R^{3a})—, —N(R^{3a})—C(O)₂—, —S(O)—, —S(O)₂—, —S(O)₂—, and —N(R^{3a})—S(O)₂—;
X in each occurrence is independently selected from C₁₋₆alkylene, C₂₋₆alkenylene, and C₂₋₆alkynylene, wherein said C₁₋₆alkylene, C₂₋₆alkenylene, and C₂₋₆alkynylene, in addition to the R⁵ to which they are attached, in each occurrence are optionally and independently substituted with one or more R⁴⁰;
Ring A is a 5- to 7-membered non-aromatic heterocyclic ring, wherein
1) said 5- to 7-membered heterocyclic ring optionally contains, in addition to the nitrogen, a member selected from —O—, —NH—, and —S—;
2) said 5- to 7-membered heterocyclic ring is optionally substituted on carbon with one or more R⁷;
3) two R⁷ substituents on one carbon atom may together optionally form the group ═O or the group ═N(OR^{7a}); and
4) if said 5- to 7-membered heterocyclic ring contains an —NH— moiety, that nitrogen is optionally substituted with R⁷*; and
n is 1 to 4.

2. A compound of Formula (I), or a pharmaceutically acceptable salt thereof, as claimed in claim 1, wherein
R¹ and R² are C₁₋₆alkyl.

3. A compound of Formula (I), or a pharmaceutically acceptable salt thereof, as claimed in claim 1, wherein
R³ in each occurrence is independently selected from —X—R⁵, —W—R⁶, —C(R^{3a})═N—R^{3y}, —C(R^{3a})═N—N(R^{3a})—C(O)—R^{3b}, —C(R^{3a})═N—N(R^{3a})—C(O)₂—R^{3b}, —C(R^{3a})═N—N(R^{3y})₂, —C(R^{3a})═N—N(R^{3a})—C(O)—N(R^{3y})₂, and —N(R^{3a})—C(O)—N(R^{3y})₂;
R^{3a} in each occurrence is independently selected from H and C₁₋₆alkyl;
R^{3b} in each occurrence is independently selected from C₁₋₆alkyl and carbocyclyl, wherein said C₁₋₆alkyl and carbocyclyl in each occurrence are optionally and independently substituted on carbon with one or more R³⁰;
R^{3y} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰;
R⁵ in each occurrence is independently selected from heterocyclyl and —Si(R^{5b})₃, wherein said heterocyclyl is optionally substituted on carbon with one or more R⁵⁰, and wherein if said heterocyclyl contains an —NH-moiety, that nitrogen in each occurrence is optionally and independently substituted with R⁵⁰*;
R^{5b} is C₁₋₆alkyl;
R⁶ is non-aromatic heterocyclyl, wherein if said non-aromatic heterocyclyl contains an —NH— moiety, that nitrogen in each occurrence is optionally and independently substituted with R⁶⁰*;
R³⁰ in each occurrence is independently selected from —CN, C₁₋₆alkyl, and —OR^{30a};
R^{30a} is C₁₋₆alkyl;
R⁵⁰ in each occurrence is independently selected from C₁₋₆alkyl and heterocyclyl;
R⁵⁰* is C₁₋₆alkyl;
R⁶⁰* in each occurrence is independently selected from C₁₋₆alkyl heterocyclyl and —C(O)₂R^{60c};
R^{60c} is C₁₋₆alkyl;
W in each occurrence is independently selected from —N(R^{3a})—C(O)—, —C(O)—N(R^{3a})—, and —N(R^{3a})—S(O)₂—; and
X is C₂₋₆alkynylene.

4. A compound of Formula (I), or a pharmaceutically acceptable salt thereof, as claimed in claim 1, wherein R⁴ in each occurrence is independently selected from H and halo.

5. A compound of Formula (I), or a pharmaceutically acceptable salt thereof, as claimed in claim 1, wherein n is 1.

6. A compound of Formula (I), or a pharmaceutically acceptable salt thereof, as claimed in claim 1, wherein
Ring A is a morpholine ring, wherein said morpholine ring is optionally substituted on carbon with one or more R⁷; and
R⁷ in each occurrence is C₁₋₆alkyl.

7. A compound of Formula (I): 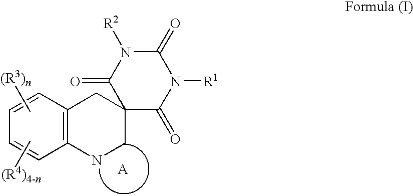
or a pharmaceutically acceptable salt thereof, wherein:
R¹ is H;
R² is H;
R³ in each occurrence is independently selected from —X—R⁵, —W—R⁶, —C(R^{3a})═N—R^{3y}, —C(R^{3a})═N—N(H)—C(O)—R^{3b}, —C(R^{3a})═N—N(H)—C(O)₂—R^{3b}, —C(R^{3a})═N—N(R^{3y})₂, —C(R^{3a})═N—N(H)—C(O)—N(R^{3y})₂, and —N(H)—C(O)—N(R^{3y})₂;
R^{3a} in each occurrence is independently selected from H and methyl;
R^{3b} in each occurrence is independently selected from methyl, t-butyl, and cyclopropyl, wherein said methyl, t-butyl, and cyclopropyl in each occurrence are optionally and independently substituted on carbon with one or more R³⁰;
R^{3y} in each occurrence is independently selected from H, 2,4-dioxoimidazolidinyl, ethyl, methyl, morpholinyl, phenyl, pyrazinyl, pyridinyl, pyrimidinyl, pyrrolidinyl, and 4H-1,2,4-triazolyl, wherein said 2,4-dioxoimidazolidinyl, morpholinyl, phenyl, pyrazinyl, pyridinyl, pyrimidinyl, pyrrolidinyl, and 4H-1,2,4-triazolyl in each occurrence are optionally and independently substituted on carbon with one or more methyl;
R⁴ in each occurrence is independently selected from H and fluoro;
R⁵ in each occurrence is independently selected from —Si(Me)₃, 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl, wherein said 1,3-benzothiazolyl, 1-benzothiophenyl, 1,3-benzoxazolyl, imidazolyl, pyrazinyl, pyridinyl, pyrimidinyl, 1,3,4-thiadiazolyl, thiazolyl, and thiophenyl are optionally substituted on carbon with one or more R⁵⁰, and wherein the —NH— nitrogen of said imidazolyl, in each occurrence is optionally and independently substituted with methyl;
R⁶ in each occurrence is independently selected from dioxidotetrahydrothiophenyl, morpholinyl, oxoimidazolidinyl, 2-oxotetrahydrofuranyl, piperidinyl, pyrrolidinyl, tetrahydrofuranyl, and tetrahydropyranyl, wherein the —NH— nitrogen of said morpholinyl, oxoimidazolidinyl, piperidinyl, and pyrrolidinyl in each occurrence is optionally and independently substituted with R⁶⁰*;
R³⁰ in each occurrence is independently selected from methyl, —CN, and methoxy;
R⁵⁰ in each occurrence is independently selected from methyl, tetrazolyl, and pyrazolyl;
R⁶⁰* in each occurrence is independently selected from methyl, pyridinyl, and —C(O)₂Me;
W in each occurrence is independently selected from —N(H)—C(O)—, —C(O)—N(H)—, and —N(H)—S(O)₂—;
X is ethyne-1,2-diyl;
Ring A is a 2,6-dimethylmorpholine ring; and
n is 1.

8-9. (canceled)

10. A method for treating a bacterial infection in a warm-blooded animal such as man, said method comprising administering to said animal an effective amount of a compound of Formula (I), as claimed in any one of claims 1 to 7 claim 1, or a pharmaceutically acceptable salt thereof.

11. (canceled)

12. A pharmaceutical composition comprising a compound of Formula (I), or a pharmaceutically acceptable salt thereof, as claimed in claim 1, and at least one pharmaceutically acceptable carrier, diluent, or excipient.

13. A process for preparing a compound of Formula (I), or a pharmaceutically acceptable salt thereof, as claimed in claim 1, said process comprising reacting a compound of Formula (A1): 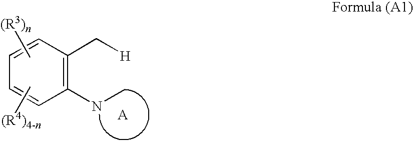
with a compound of Formula (A2): 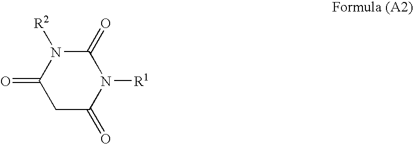
and thereafter if necessary:
i) converting a compound of Formula (I) into another compound of Formula (I);
ii) removing any protecting groups; and/or
iii) forming a pharmaceutically acceptable salt.
